(19) United States
(12) Patent Application Publication
(10) Pub. No.: US 2024/0002928 A1
(43) Pub. Date: Jan. 4, 2024

(54) HIGH-THROUGHPUT NUCLEIC ACID SEQUENCING WITH SINGLE-MOLECULE-SENSOR ARRAYS

(71) Applicants: Roche Sequencing Solutions, Inc., Pleasanton, CA (US); Western Digital Technologies, Inc., San Jose, CA (US)

(72) Inventors: Juraj TOPOLANCIK, Redwood City, CA (US); Patrick BRAGANCA, San Jose, CA (US); Yann ASTIER, Pleasanton, CA (US); Sri PALADUGU, Mountain House, CA (US)

(73) Assignees: Roche Sequencing Solutions, Inc., Pleasanton, CA (US); Western Digital Technologies, Inc., San Jose, CA (US)

(21) Appl. No.: 17/996,360

(22) PCT Filed: Apr. 21, 2021
(86) PCT No.: PCT/US2021/028263

§ 371 Date: Oct. 17, 2022

Related U.S. Application Data
(60) Provisional application No. 63/013,236, filed on Apr. 21, 2020.

(51) Int. Cl. C12Q 1/6874; B01L 3/00; G16B 40/10

(52) U.S. Cl. CPC C12Q 1/6874; B01L 3/502761; G16B 40/10; B01L 2300/0819; B01L 2200/16; B01L 2200/0652; C12Q 2600/16

(57) ABSTRACT

Disclosed herein are embodiments of single-molecule array sequencing (SMAS) devices and systems. Each sensor of an array of sensors of the SMAS device is capable of detecting labels attached to nucleotides incorporated into a single nucleic acid strand bound to a respective binding site. Each sensor can detect a single label (e.g., fluorescent, magnetic, organometallic, charged molecule, etc.) attached to the incorporated nucleotide. Also disclosed are methods of using SMAS devices and systems for highly-scalable nucleic acid (e.g., DNA) sequencing based on sequencing by synthesis (SBS) of multiple instances of clonally amplified DNA immobilized on such SMAS devices. Also disclosed are error correction methods that mitigate errors (e.g., errant label detections or non-detections) made in sequencing individual nucleic acid strands.

### CROSS-REFERENCE TO RELATED APPLICATIONS

This application claims priority to, and hereby incorporates by reference in its entirety the contents of, U.S. provisional application No. 63/013,236, filed Apr. 21, 2020 and entitled “HIGH-THROUGHPUT DNA SEQUENCING WITH SINGLE-MOLECULE SENSOR-ARRAYS” (Attorney Docket No. ROA-1002P-US/P36083-US). This application also incorporates by reference for all purposes the entireties of PCT application No. PCT/US20/27290, filed Apr. 8, 2020, entitled “NUCLEIC ACID SEQUENCING BY SYNTHESIS USING MAGNETIC SENSOR ARRAYS” (Attorney Docket No. ROA-1000-WO/P35097-WO), which published on Oct. 15, 2020 as WO 2020/210370, and PCT Application No. PCT/US2021/021274, filed Mar. 7, 2021 and entitled “MAGNETIC SENSOR ARRAYS FOR NUCLEIC ACID SEQUENCING AND METHODS OF MAKING AND USING THEM” (Attorney Docket No. ROA-1001-WO/P35967-WO).

### SEQUENCE LISTING

The instant application contains a Sequence Listing that has been submitted electronically in ASCII format and is hereby incorporated by reference in its entirety. Said ASCII copy, created on Jul. 19, 2023, is named ROA-1002-US P36083-US-1_SL.txt and is 3,232 bytes in size.

### BACKGROUND

Commercially-successful approaches to DNA sequencing involve either synthesis and analysis of clonal deoxyribonucleic acid (DNA) clusters or detection of individual DNA molecules. Although cluster sequencers exhibit error rates that are sufficiently low for diagnostic applications, they are quite limited in read length due to the nature of error propagation in molecular ensembles. Single-molecule sequencers can generate considerably longer reads, but often exhibit static and dynamic heterogeneity that results in errors that are too large for high-precision diagnostics.

Thus, there is a need to improve DNA sequencing, and nucleic acid sequencing in general, to enable longer reads with lower error rates.

### SUMMARY

This summary represents non-limiting embodiments of the disclosure.

Disclosed herein are embodiments of single-molecule array sequencing (SMAS) devices and systems. Each sensor of a plurality of sensors within an array of sensors of the SMAS device detects labels attached to nucleotides incorporated into a single nucleic acid strand bound to a respective binding site. Each sensor can detect a single label (e.g., fluorescent, magnetic, organometallic, charged molecule, etc.) attached to the incorporated nucleotide. Also disclosed are methods of using SMAS devices and systems for highly-scalable nucleic acid (e.g., DNA) sequencing based on sequencing by synthesis (SBS) of multiple instances of clonally amplified DNA immobilized on such SMAS devices. Also disclosed are error correction methods that mitigate errors (e.g., errant label detections or non-detections) made in sequencing individual nucleic acid strands.

In some embodiments, a device for sequencing nucleic acid comprises a fluid chamber, a plurality of S magnetic sensors configured to detect labels present in the fluid chamber, and at least one processor. The fluid chamber comprises a plurality of S binding sites, each of the S binding sites configured to bind no more than one strand of nucleic acid. Each of the S magnetic sensors senses a respective strand of nucleic acid bound to a respective binding site of the S binding sites. The at least one processor is configured to execute one or more machine-executable instructions that, when executed, cause the at least one processor to, at each inquiry step of a plurality of M inquiry steps of a sequencing procedure, and for each of the S magnetic sensors, (a) obtain a respective characteristic of the respective magnetic sensor, wherein the respective characteristic indicates presence or absence of at least one label, and (b) based at least in part on the obtained respective characteristic, determine whether the respective magnetic sensor detected the presence or absence of at least one label during the inquiry step.

In some embodiments, a system comprises a plurality of S binding sites, each of the S binding sites configured to bind no more than one strand of nucleic acid, a plurality of S sensors (e.g., magnetic, optical, etc.) configured to detect labels, and at least one processor. Each of the S sensors is configured to sense a respective strand of nucleic acid bound to a respective binding site of the S binding sites. The at least one processor is configured to execute one or more machine-executable instructions that, when executed, cause the at least one processor to, at each inquiry step of a plurality of M inquiry steps of a sequencing procedure, and for each of the S sensors, (a) obtain a respective characteristic of the respective sensor, wherein the respective characteristic indicates presence or absence of at least one label, and (b) based at least in part on the obtained respective characteristic, determine whether the respective sensor detected the presence or absence of at least one label during the inquiry step. In addition, when executed, the one or more machine-executable instructions further cause the at least one processor to perform an error-correction procedure on at least one record, the at least one record comprising results of the sequencing procedure for at least a subset of the S sensors at each of the M inquiry steps.

In some embodiments, a method of sequencing a plurality of S nucleic acid strands using a SMAS device comprises (a) binding the S nucleic acid strands to the S binding sites, (b) performing a sequencing procedure comprising M inquiry steps to produce S records, each of the S records capturing M detection results of a respective one of the S sensors, each of the M detection results indicating whether, during a respective one of the M inquiry steps, the respective one of the S sensors detected at least one label in the fluid chamber, and (c) applying an error correction procedure to at least a subset of the S records to estimate a nucleic acid sequence of at least one of the S nucleic acid strands.

Some embodiments are a method of mitigating errors in sequencing data generated as a result of a nucleic acid sequencing procedure using a single-molecule sensor array, the single-molecule sensor array having a plurality of sensors, each of the plurality of sensors associated with a respective binding site of a plurality of binding sites, each of the plurality of binding sites configured to bind no more than one strand of nucleic acid to be sequenced. In some such embodiments, the method comprises (a) identifying, in the sequencing data, a plurality of records, each of the plurality of records capturing a respective sequencing result for a respective instance of a first strand of nucleic acid, each of the plurality of records having a plurality of entries, each of the plurality of entries indicating, for a respective one of a plurality of inquiry steps of the nucleic acid sequencing procedure, that either (i) a label was detected by a respective sensor associated with the respective instance of the first strand of nucleic acid, or (ii) no label was detected by the respective sensor associated with the respective instance of the first strand of nucleic acid; (b) based on the plurality of records, determining a plurality of candidate sequences for the first strand of nucleic acid, each of the plurality of candidate sequences estimating at least a portion of a nucleic acid sequence of the first strand of nucleic acid; and (c) identifying, as the at least a portion the nucleic acid sequence of the first strand of nucleic acid, a particular candidate sequence of the plurality of candidate sequences that is, from among the plurality of candidate sequences, most likely to be correct.

The disclosed sequencing and error correction devices, systems, and methods promise potentially higher throughput, lower error rates, and longer read lengths compared to cluster-based approaches.

### BRIEF DESCRIPTION OF THE DRAWINGS

Objects, features, and advantages of the disclosure will be readily apparent from the following description of certain embodiments taken in conjunction with the accompanying drawings in which:

FIG. 1 illustrates a portion of a magnetic sensor in accordance with some embodiments.

FIGS. 2A and 2B illustrate the resistance of magneto-resistive (MR) sensors, which may be used in accordance with some embodiments.

FIG. 3A illustrates a spin-torque oscillator (STO) sensor, which may be used in accordance with some embodiments.

FIG. 3B shows the experimental response of a STO under example conditions.

FIGS. 3C and 3D illustrate short nanosecond field pulses of STOs that may be used in accordance with some embodiments.

FIG. 4A illustrates a single sensor of a cluster sequencing device used to sense some number N of clonally-amplified DNA strands in its vicinity.

FIG. 4B illustrates an exemplary plurality of S single-molecule sensors, each used by a SMAS device to monitor a respective single-stranded DNA (ssDNA) in accordance with some embodiments.

FIG. 5A is a block diagram showing components of an exemplary SMAS device for nucleic acid sequencing in accordance with some embodiments.

FIGS. 5B, 5C, and 5D illustrate portions of an exemplary SMAS device for nucleic acid sequencing in accordance with some embodiments.

FIG. 5E illustrates a square grid (or lattice) pattern of sensors in accordance with some embodiments.

FIG. 6A illustrates a sensor, a DNA strand in a coiled state, and a label in accordance with some embodiments.

FIG. 6B illustrates exemplary dimensions of a sensor, an elongated DNA strand, and a label in accordance with some embodiments.

FIG. 7A illustrates an exemplary geometrical arrangement for estimating the sensor-array packing limit of a SMAS device in accordance with some embodiments.

FIG. 7B illustrates sensors of a SMAS device arranged in a square lattice in accordance with some embodiments.

FIGS. 8A and 8B illustrate sensors of a SMAS device arranged in a hexagonal pattern in accordance with some embodiments.

FIG. 9A illustrates an exemplary geometrical arrangement for estimating the sensor-array packing limit of a SMAS device in accordance with some embodiments.

FIG. 9B illustrates sensors of a SMAS device arranged in a hexagonal lattice in accordance with some embodiments.

FIG. 10 compares the densities of exemplary SMAS implementations to state-of-the-art cluster sequencing devices.

FIG. 11 illustrates an exemplary method of sequencing a plurality of nucleic acid strands using a SMAS device in accordance with some embodiments.

FIG. 12 is a flow diagram of a sequencing procedure using an additive approach in accordance with some embodiments.

FIG. 13 illustrates an additive sequencing protocol in accordance with some embodiments.

FIG. 14 is a flow diagram of a sequencing procedure using a subtractive approach in accordance with some embodiments.

FIG. 15 illustrates a subtractive sequencing protocol in accordance with some embodiments.

FIG. 16 is a flow diagram of a sequencing procedure using a modified additive approach in accordance with some embodiments.

FIG. 17 illustrates a modified additive sequencing protocol in accordance with some embodiments.

FIG. 18A illustrates failed nucleotide incorporation (FNI) for a cluster sequencing device.

FIG. 18B illustrates FNI for a SMAS device.

FIG. 18C illustrates failed label removal (FLR) for a cluster sequencing device.

FIG. 18D illustrates FLR for a SMAS device.

FIG. 18E illustrates failed nucleotide removal (FNR) for a cluster sequencing device.

FIG. 18F illustrates FNR for a SMAS device.

FIG. 18G illustrates failed nucleotide detection (FLD) for a cluster sequencing device.

FIG. 18H illustrates FLD for a SMAS device.

FIG. 19 is a flow diagram of an exemplary sequencing procedure using the modified additive approach with FLR and FNI error detection in accordance with some embodiments.

FIG. 20 shows example records with FNI and FLR errors.

FIG. 21 illustrates the expected signal level detected by a cluster sequencing device sensor capturing the behavior of the molecular ensemble during the sequencing procedure.

FIG. 22 illustrates how SMAS devices provide better accuracy when using error-correction techniques in accordance with some embodiments.

FIG. 23 illustrates the correction of FNI errors by deleting runs of four “no label detected” entries in records of detection results from the sequencing procedure in accordance with some embodiments.

FIG. 24 illustrates the results of exemplary SBS reactions in accordance with some embodiments.

FIG. 25 illustrates the effect of larger cluster size on the base-calling accuracy of a cluster sequencing device.

FIG. 26 illustrates deterministic error correction of FLR and FNI errors in accordance with some embodiments.

FIG. 27 illustrates FNI, FLR, and FNR errors in detection data.

FIG. 28 illustrates FLR error correction and base-calling from data produced by a SMAS device in accordance with some embodiments.

FIG. 29 illustrates FNI error correction and base-calling from data produced by a SMAS device in accordance with some embodiments.

FIG. 30 illustrates error correction and base-calling from data produced by a SMAS device in accordance with some embodiments.

FIG. 31 illustrates FNI, FLR, FNR, and FLD errors in exemplary detection results from a SMAS device.

FIG. 32 illustrates the application of error-correction procedures to the data captured during SBS by a SMAS device in accordance with some embodiments.

FIG. 33 is a flow diagram illustrating an error-correction procedure in accordance with some embodiments.

FIG. 34A illustrates the average signal intensity at an inquiry step at which labels should be detected because matching nucleotides are introduced and successfully incorporated.

FIG. 34B illustrates function fit to the measured intensities from a cluster model.

FIG. 35 plots probability functions for a cluster sequencing device.

FIG. 36 illustrates the discrete probability functions for a cluster sequencing device.

FIG. 37A illustrates intensity plots of a cluster sequencing device.

FIG. 37B illustrates a probability distribution function for a cluster sequencing device.

FIGS. 38A and 38B plot probability functions for a cluster sequencing device.

FIG. 39 illustrates the N-r parameter space of a cluster sequencing device under various conditions.

FIG. 40A shows the calculated probability for a cluster sequencing device along the Q30 contour for various N-r combinations.

FIG. 40B plots calculated cumulative error probabilities for a cluster sequencing device.

FIG. 41 illustrates the N-r parameter space for a cluster sequencing device where the cumulative probabilities of an incorrect base-call at position 150 are less than or equal to 1 in 100 (\tilde{Q}20), 1 in 1,000 (\tilde{Q}30), 1 in 10,000 (\tilde{Q}40), and 1 in 100,000 (\tilde{Q}50).

FIG. 42 illustrates the calculated results for the K-r parameter space for a SMAS device where the probability of an incorrect base-call at every inquiry step is lower than 1 in 100 (Q20), 1 in 1,000 (Q30), 1 in 10,000 (Q40) and 1 in 100,000 (Q50) in accordance with some embodiments.

FIGS. 43A and 43B show the cumulative probabilities of an incorrect base-call at position 150 for cluster sequencing devices and SMAS devices in accordance with some embodiments.

FIGS. 44 and 45 illustrate an exemplary sample preparation and loading process in accordance with some embodiments.

FIGS. 46A, 46B, and 46C illustrate simulated detection results for an exemplary SMAS device in accordance with some embodiments.

FIG. 47 illustrates how the detection data illustrated in FIGS. 46A, 46B, and 46C can be rearranged to call bases and reveal the positions of different DNA strands in accordance with some embodiments.

FIGS. 48A and 48B plot the calculated probability of making an incorrect base-call as a function of the inquiry step number C and chemistry failure rate r.

FIG. 49 illustrates the use of barcodes in sample preparation and DNA loading in accordance with some embodiments.

FIG. 50 illustrates an exemplary system 160 in accordance with some embodiments.

To facilitate understanding, identical reference numerals have been used, where possible, to designate identical elements that are common to the figures. It is contemplated that elements disclosed in one embodiment may be beneficially utilized in other embodiments without specific recitation. Moreover, the description of an element in the context of one drawing is applicable to other drawings illustrating that element.

### DETAILED DESCRIPTION

Some descriptions and examples herein are in the context of DNA sequencing, but it is to be appreciated that the disclosures apply generally to nucleic acid sequencing.

### Terminology and Notation

As used herein, the term “strand” refers to a single nucleic acid strand (e.g., ssDNA). The terms “strands” and “fragments” are used interchangeably when referring to nucleic acids.

As used herein, the term “plurality” means two or more, but not necessarily all. Thus, a plurality of sensors means only at least two sensors, but not necessarily all sensors in the sensor array or sequencing device/system. Likewise, a plurality of binding sites means only at least two binding sites, not necessarily all binding sites in the sequencing device/system.

As used herein, the term “instance” when referring to nucleic acid strands means a template nucleic acid strand or a copy thereof (e.g., produced by an amplification or replication process). Ideally, copies of a template nucleic acid strand are identical to the template strand, but, as is known in the art, copies are not necessarily identical due to replication/amplification errors. It will be appreciated that replicates produced by amplification are still considered copies of the original nucleic acid strand even if the amplification procedure introduces errors. Thus, all instances of a strand are ideally identical to each other but might not be.

As used herein, the term “inquiry cycle” refers to a single cycle of a nucleic acid sequencing procedure during which all possible nucleotides are introduced to determine which, if any, is incorporated into a strand being sequenced. For example, for DNA sequencing procedures, all of adenine (A), thymine (T), cytosine (C), and guanine (G) are tested in some (arbitrary) order (which need not be the same from inquiry cycle to inquiry cycle). As explained in detail below, depending on the selected sequencing procedure, more than one label may be detected per strand during a single sequencing cycle.

As used herein, the term “inquiry step” refers to a step or collection of steps of the sequencing procedure during which it is determined whether one or more sensors of a sequencing device are detecting labels. For DNA sequencing cycling through all of A, T, C, and G, there are four inquiry steps per inquiry cycle (one for each nucleotide). For a sensor in use, each inquiry step results in a single determination of whether that sensor is or is not detecting a label.

As used herein, the term “detection result” refers to a value indicating either (a) a label was detected during an inquiry step or (b) no label was detected during the inquiry step. In some embodiments, the detection results are binary values (e.g., 0 or 1). Detection results may be derived from other data (e.g., a signal representing resistance, frequency, intensity, etc.; a measurement of resistance, frequency, intensity, etc.).

As used herein, the term “record” refers to a stored representation of the detection result(s) for a single sensor. If the selected sequencing procedure has M inquiry steps, then upon completion of the sequencing procedure, each record has M detection results. Records of S sensors may be stored in a single file (e.g., as a table having S rows and M columns, or S columns and M rows), or separate files may be created for respective sensors' records.

As used herein in reference to the detection results contained within a record, the term “run” means a sequence of consecutive identical values.

The terms “sensor” and “sensing element” are used interchangeably herein.

The variable S is used herein to refer to a number of sensors in a plurality of sensors. The S sensors may be sensing instances of the same strand, or they may be sensing instances of different strands.

The variable K is used herein to refer to a number of sensors in a plurality of sensors that all sense instances of the same strand.

### Labels

Methods for nucleic acid sequencing described herein use labeled nucleotide precursors comprising cleavable labels. These cleavable labels may be, for example, magnetic, fluorescent, organometallic, or charged molecules.

Each label may comprise, for example, a magnetic nanoparticle, such as, for example, a molecule, a superparamagnetic nanoparticle, or a ferromagnetic particle. The magnetic labels may be nanoparticles with high magnetic anisotropy. Examples of nanoparticles with high magnetic anisotropy include, but are not limited to, Fe₃O₄, FePt, FePd, and CoPt. To facilitate chemical binding to nucleotides, the particles may be synthesized and coated with SiO₂. See, e.g., M. Aslam, L. Fu, S. Li, and V. P. Dravid, “Silica encapsulation and magnetic properties of FePt nanoparticles,” Journal of Colloid and Interface Science, Volume 290, Issue 2, 15 Oct. 2005, pp. 444-449. Because magnetic labels of this size have permanent magnetic moments, the directions of which fluctuate randomly on very short time scales, some embodiments, described further below, rely on sensitive sensing schemes that detect fluctuations in magnetic field caused by the presence of the magnetic labels.

Each label may comprise, for example, a fluorophore. Fluorescent labels are well known in the art and are suitable for use with the disclosures herein.

The labels may comprise, for example, organometallic compounds. As will be appreciated, an organometallic compound is any member of a class of substances containing at least one metal-to-carbon bond in which the carbon is part of an organic group. Examples of organometallic compounds include Gilman reagents (which contain lithium and copper), Grinard reagents (which contain magnesium), tetracarbonyl nickel and ferrocene (which contain transition metals), organolithium compounds (e.g., n-butyllithium (n-BuLi)), organozinc compounds (e.g., diethylzinc (Et₂Zn)), organotin compounds (e.g., tributyltin hydride(Bu₃SnH)), organoborane compounds (e.g., triethylborane (Et₃B)), and organoaluminium compounds (e.g., trimethylaluminium (Me₃Al)).

The labels may comprise, for example, charged molecules.

There are a number of ways to attach the labels to nucleotide precursors and to cleave the labels after incorporation of the nucleotide precursor. For example, the labels may be attached to a base, in which case they may be cleaved chemically. As another example, the labels may be attached to a phosphate, in which case they may be cleaved by polymerase or, if attached via a linker, by cleaving the linker.

In some embodiments, the label is linked to the nitrogenous base (e.g., A, C, T, G, or a derivative) of the nucleotide precursor. After incorporation of the nucleotide precursor and the detection by a sequencing device (e.g., as described in further detail below), the label is cleaved from the incorporated nucleotide.

In some embodiments, the label is attached via a cleavable linker. Cleavable linkers are known in the art and have been described, e.g., in U.S. Pat. Nos. 7,057,026, 7,414,116 and continuations and improvements thereof. In some embodiments, the label is attached to the 5-position in pyrimidines or the 7-position in purines via a linker comprising an allyl or azido group. In other embodiments, the linker comprises a disulfide, indole or a Sieber group. The linker may further contain one or more substituents selected from alkyl (C₁₋₆) or alkoxy (C₁₋₆), nitro, cyano, fluoro groups or groups with similar properties. Briefly, the linker can be cleaved by water-soluble phosphines or phosphine-based transition metal-containing catalysts. Other linkers and linker cleavage mechanisms are known in the art. For example, linkers comprising trityl, p-alkoxybenzyl esters and p-alkoxybenzyl amides and tert-butyloxycarbonyl (Boc) groups and the acetal system can be cleaved under acidic conditions by a proton-releasing cleavage agent. A thioacetal or other sulfur-containing linker can be cleaved using a thiophilic metals, such as nickel, silver or mercury. The cleavage protecting groups can also be considered for the preparation of suitable linker molecules. Ester- and disulfide containing linkers can be cleaved under reductive conditions. Linkers containing triisopropyl silane (TIPS) or t-butyldimethyl silane (TBDMS) can be cleaved in the presence of F ions. Photocleavable linkers cleaved by a wavelength that does not affect other components of the reaction mixture include linkers comprising O-nitrobenzyl groups. Linkers comprising benzyloxycarbonyl groups can be cleaved by Pd-based catalysts.

In some embodiments, the nucleotide precursor comprises a label attached to a polyphosphate moiety as described in, e.g., U.S. Pat. Nos. 7,405,281 and 8,058,031. Briefly, the nucleotide precursor comprises a nucleoside moiety and a chain of 3 or more phosphate groups where one or more of the oxygen atoms are optionally substituted, e.g., with S. The label may be attached to the a, (3, y or higher phosphate group (if present) directly or via a linker. In some embodiments, the label is attached to a phosphate group via a non-covalent linker as described, e.g., in U.S. Pat. No. 8,252,910. In some embodiments, the linker is a hydrocarbon selected from substituted or unsubstituted alkyl, substituted or unsubstituted heteroalkyl, substituted or unsubstituted aryl, substituted or unsubstituted heteroaryl, substituted or unsubstituted cycloalkyl, and substituted or unsubstituted heterocycloalkyl; see, e.g., U.S. Pat. No. 8,367,813. The linker may also comprise a nucleic acid strand; see, e.g., U.S. Pat. No. 9,464,107.

In embodiments in which the label is linked to a phosphate group, the nucleotide precursor is incorporated into the nascent chain by the nucleic acid polymerase, which also cleaves and releases the detectable label. In some embodiments, the label is removed by cleaving the linker, e.g., as described in U.S. Pat. No. 9,587,275.

In some embodiments, the nucleotide precursors are non-extendable “terminator” nucleotides, i.e., the nucleotides that have a 3′-end blocked from addition of the next nucleotide by a blocking “terminator” group. The blocking groups are reversible terminators that can be removed in order to continue the strand synthesis process as described herein. Attaching removable blocking groups to nucleotide precursors is known in the art. See, e.g., U.S. Pat. Nos. 7,541,444, 8,071,739 and continuations and improvements thereof. Briefly, the blocking group may comprise an allyl group that can be cleaved by reacting in aqueous solution with a metal-allyl complex in the presence of phosphine or nitrogen-phosphine ligands. Other examples of reversible terminator nucleotides used in sequencing by synthesis include the modified nucleotides described in International Application No. PCT/US2019/066670, filed Dec. 16, 2019 and entitled “3′-protected Nucleotides,” which published as WO/2020/131759.

### Sensors

The characteristics and capabilities of sensors used in the nucleic acid sequencing devices, systems, and methods described herein depend on the choice of labels used. The sensors may be, for example, magnetic sensors (to detect, e.g., magnetic nanoparticles, organometallic compounds, etc.) or optical sensors (to detect, e.g., fluorophores). It is to be appreciated that other types of sensors may be suitable to detect labels of various types, and the examples described herein are not intended to be limiting. Generally speaking, the disclosed devices, systems, and methods can use any kind of label that can be detected by the selected type of sensor, and, conversely, the disclosed devices, systems, and methods can use any kind of sensor that can detect the presence (and absence) of the selected type of label.

The reference number 105 is used herein for single-molecule sensors generally, regardless of the type of those single-molecule sensors (and regardless of the type of label they detect). The reference number 15 is used for sensors that sense clusters of nucleic acid strands.

Magnetic Sensors

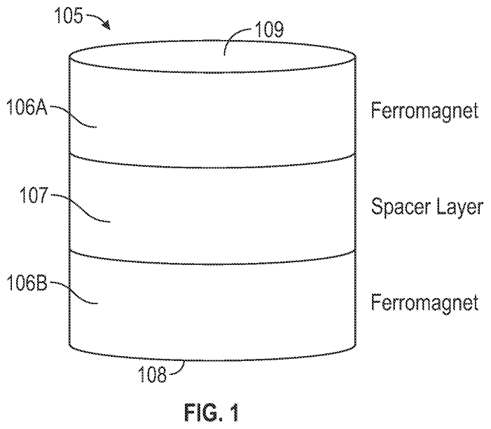

Some embodiments disclosed herein use magnetic sensors to detect the presence of magnetic labels (e.g., magnetic nanoparticles, organometallic complexes, charged molecules, etc.) coupled to nucleotide precursors. FIG. 1 illustrates a portion of a magnetic sensor 105 in accordance with some embodiments. The exemplary magnetic sensor 105 of FIG. 1 has a bottom surface 108 and a top surface 109 and comprises three layers, e.g., two ferromagnetic layers 106A, 106B separated by a nonmagnetic spacer layer 107. The nonmagnetic spacer layer 107 may be, for example, a metallic material such as, for example, copper or silver, in which case the structure is called a spin valve (SV), or it may be an insulator such as, for example, alumina or magnesium oxide, in which case the structure is referred to as a magnetic tunnel junction (MTJ). Suitable materials for use in the ferromagnetic layers 106A, 106B include, for example, alloys of Co, Ni, and Fe (sometimes mixed with other elements). In some embodiments, the ferromagnetic layers 106A, 106B are engineered to have their magnetic moments oriented either in the plane of the film or perpendicular to the plane of the film. Additional materials may be deposited both below and above the three layers 106A, 106B, and 107 shown in FIG. 1 to serve purposes such as interface smoothing, texturing, and protection from processing used to pattern the device into which the sensor 105 is incorporated, but the active region of the magnetic sensor 105 lies in this trilayer structure. Thus, a component that is in contact with a magnetic sensor 105 may be in contact with one of the three layers 106A, 106B, or 107, or it may be in contact with another part of the magnetic sensor 105.

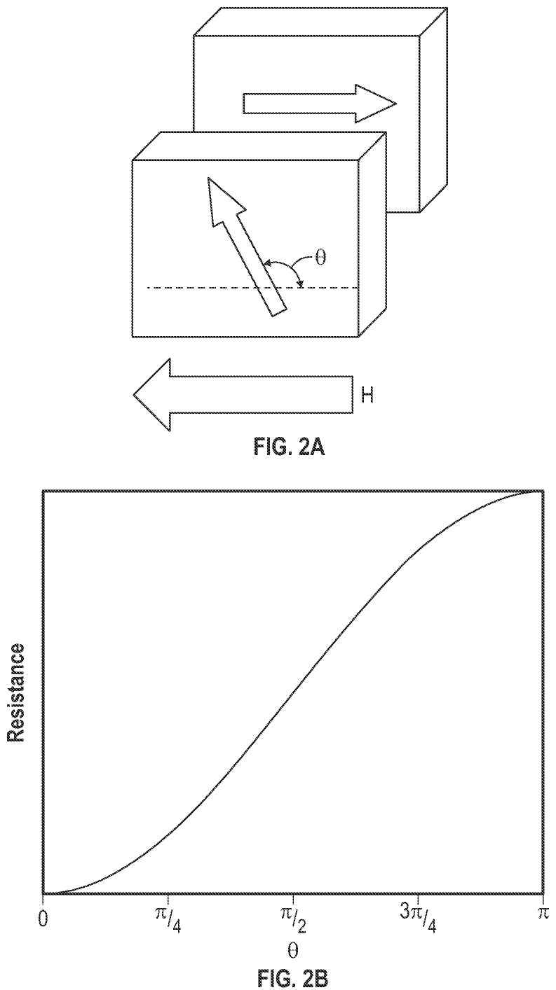

As shown in FIGS. 2A and 2B, the resistance of MR sensors is proportional to 1−cos(θ), where θ is the angle between the moments of the two ferromagnetic layers 106A, 106B shown in FIG. 1. To maximize the signal generated by a magnetic field and provide a linear response of the magnetic sensor 105 to an applied magnetic field, the magnetic sensor 105 may be designed such that the moments of the two ferromagnetic layers 106A, 106B are oriented π/2 radians or 90 degrees with respect to one another in the absence of a magnetic field. This orientation can be achieved by any number of methods that are known in the art. For example, one solution is to use an antiferromagnet to “pin” the magnetization direction of one of the ferromagnetic layers (either 106A or 106B, designated as “FM1”) through an effect called exchange biasing and then coat the sensor with a bilayer that has an insulating layer and permanent magnet. The insulating layer avoids electrical shorting of the magnetic sensor 105, and the permanent magnet supplies a “hard bias” magnetic field perpendicular to the pinned direction of FM1 that will then rotate the second ferromagnet (either 106B or 106A, designated as “FM2”) and produce the desired configuration. Magnetic fields parallel to FM1 then rotate FM2 about this 90 degree configuration, and the change in resistance results in a voltage signal that can be calibrated to measure the field acting upon the magnetic sensor 105. In this manner, the magnetic sensor 105 acts as a magnetic-field-to-voltage transducer.

Note that although the example discussed immediately above described the use of ferromagnets that have their moments oriented in the plane of the film at 90 degrees with respect to one another, a perpendicular configuration can alternatively be achieved by orienting the moment of one of the ferromagnetic layers 106A, 106B out of the plane of the film, which may be accomplished using what is referred to as perpendicular magnetic anisotropy (PMA).

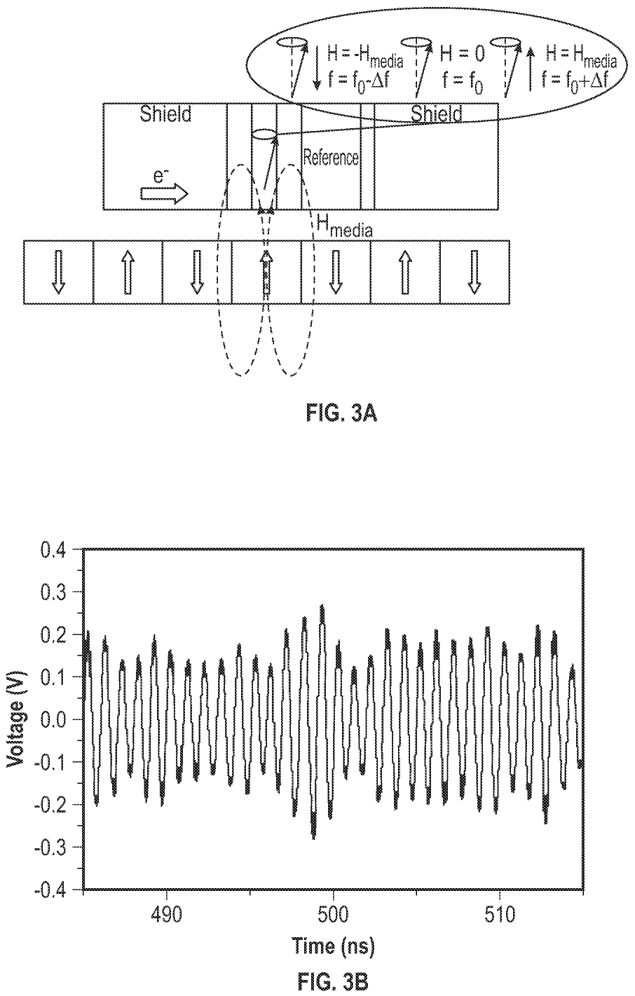

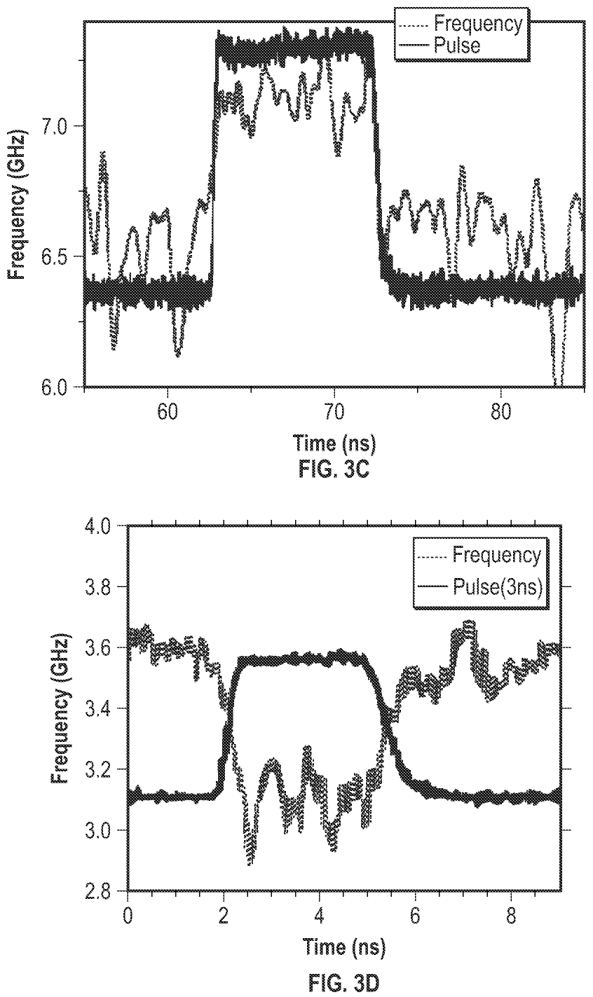

In some embodiments, the magnetic sensors 105 use a quantum mechanical effect known as spin transfer torque. In such devices, the electrical current passing through one ferromagnetic layer 106A (or 106B) in a SV or a MTJ preferentially allows electrons with spin parallel to the layer's moment to transmit through, while electrons with spin antiparallel are more likely to be reflected. In this manner, the electrical current becomes spin polarized, with more electrons of one spin type than the other. This spin-polarized current then interacts with the second ferromagnetic layer 106B (or 106A), exerting a torque on the layer's moment. This torque can in different circumstances either cause the moment of the second ferromagnetic layer 106B (or 106A) to precess around the effective magnetic field acting upon the ferromagnet, or it can cause the moment to reversibly switch between two orientations defined by a uniaxial anisotropy induced in the system. The resulting spin torque oscillators (STDs) are frequency-tunable by changing the magnetic field acting upon them. Thus, they have the capability to act as magnetic-field-to-frequency (or phase) transducers (thereby producing an AC signal having a frequency), as is shown in FIG. 3A, which illustrates the concept of using a STO sensor. FIG. 3B shows the experimental response of a STO through a delay detection circuit when an AC magnetic field with a frequency of 1 GHz and a peak-to-peak amplitude of 5 mT is applied across the STO. This result and those shown in FIGS. 3C and 3D for short nanosecond field pulses illustrate how these oscillators may be used as nanoscale magnetic field detectors. Further details may be found in T. Nagasawa, H. Suto, K. Kudo, T. Yang, K. Mizushima, and R. Sato, “Delay detection of frequency modulation signal from a spin-torque oscillator under a nanosecond-pulsed magnetic field,” Journal of Applied Physics, Vol. 111, 07C908 (2012).

Optical Sensors

Some nucleic acid sequencing approaches use fluorescent labels. In such approaches, a nucleic acid molecule being sequenced is immobilized on a solid support, and the binding of a fluorescently labeled target molecule (e.g., a nucleotide) to the molecule is monitored. An optical instrument, e.g., an excitation and reading device for fluorescence, provides light at a certain wavelength to excite the fluorescent label and detects the fluorescence light from the label emitted at a somewhat different wavelength. Because the beam path (light path) of the excitation light must at least partially differ from the beam path (light path) of the fluorescent light, spectral separation may be accomplished using excitation and emission filters (the spectra of which do not significantly overlap), and/or either vertical or side illumination may be used.

Optical sensors and sequencing devices and methods that use fluorescent labels (e.g., fluorophores) are well known in the art.

### Amplification/Replication

Nucleic acid sequencing devices generally rely on an amplification (or replication) process to generate a large number of nucleic acid instances from a single nucleic acid strand (e.g., instances of single-sided DNA strands (ssDNA) from one single DNA molecule). The polymerase chain reaction (PCR) is a well-known method for amplifying double-stranded DNA that enables replication of substantial amounts of DNA from small initial amounts.

### Cluster Sequencing Devices

Some sequencing devices, referred to herein as cluster (CLUS) devices, use amplification techniques to form a localized cluster of many DNA strands. For example, one single DNA strand is used as a template, and PCR amplification generates thousands or millions of instances of DNA sequences in a localized region. At least a part of the PCR primers are immobilized to a solid support, which allows the generated DNA molecules to be immobilized to a local cluster so as to form a distinguishable “clone.” The generated DNA cluster may comprise ssDNA. Examples of the clonal amplification techniques include bridge PCR and emulsion PCR, including bead-based emulsion PCR. For bridge amplification, a single DNA molecule is amplified to form a DNA cluster by in situ PCR using primers attached to a solid surface, such as a glass slide. Each DNA cluster is a physically separated “clone” consisting of instances of DNA strands. For emulsion PCR-based clonal amplification, single DNA molecules are clonally amplified in emulsion droplets. In some methods, DNA strands are attached to microbeads inside the droplets. The clonal amplification of single molecules can also be performed in separate micro-wells.

As used herein, the term “cluster” refers to a localized cluster of nucleic acid strands, ideally having identical sequences, which is generated from a clonal amplification. When the nucleic acid is DNA, the cluster comprises (ideally) identical DNA strands (or fragments) that are attached to a solid support. For example, the clusters can be generated on spots of a glass slide or be attached to microbeads, micro-wells, or other microparticles.

The use of CLUS devices for fluorescence-based DNA sequencing is well known.

Sequencing devices using arrays of magnetic sensors for nucleic acid sequencing using clusters are described, for example, in PCT Application No. PCT/US2021/021274, filed Mar. 7, 2021 and entitled “MAGNETIC SENSOR ARRAYS FOR NUCLEIC ACID SEQUENCING AND METHODS OF MAKING AND USING THEM” (Attorney Docket No. ROA-1001-WO/P35967-WO).

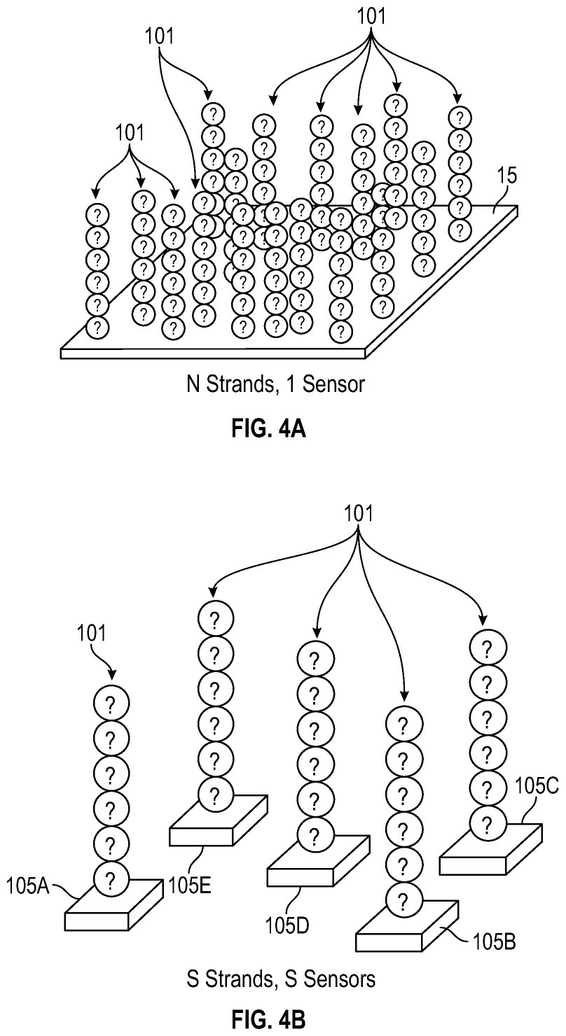

FIG. 4A illustrates a single sensor 15 of a CLUS device used to sense some number N of clonally-amplified DNA strands 101 in its vicinity. The sensor 15 may be, for example, a magnetic sensor to sense magnetic labels attached to incorporated nucleotides. For convenience, FIG. 4A shows the strands 101 in contact with the sensor 15, but it is to be appreciated that there may be a barrier (e.g., an insulating layer) between the sensor 15 and the strands 100. The sensor 15 may be, for example, a magnetic sensor as described in the above-cited PCT Application No. PCT/US2021/021274.

State-of-the-art commercial CLUS devices, such as those that sense fluorescent labels, may use hundreds of millions of sensors 15, each sensing many instances of a respective amplified DNA strand 101. One drawback of some CLUS devices is that achieving optimal cluster density can be critical to high-quality sequencing. Specifically, the use of large clusters tends to provide higher data quality, but lower data output, whereas the use of small clusters can lead to run failure, poor run performance, lower Q30 scores, introduction of sequencing artifacts, and lower total data output. To mitigate these issues, newer CLUS devices use patterned flow cells that have distinct nanowells for cluster generation. These nanowells are organized in a hexagonal arrangement to make more efficient use of the flow cell surface area.

### Single-Molecule Array Sequencing Devices

Single-molecule array sequencing devices (referred to herein as “SMAS devices”) are an alternative to CLUS devices. In contrast to CLUS devices, which sense and sequence localized clusters of multiple instances of a single nucleic acid strand, SMAS devices use sensors that individually sense and sequence individual strands of nucleic acid. Generally speaking, in SMAS devices, no sensor senses more than one physical nucleic acid strand, but different sensors sense instances of the same strand. In other words, multiple instances of a nucleic acid strand are present, but each sensed strand is sensed by a different respective sensor. Depending on the amplification technique used, the individual strands may be distributed randomly throughout a fluid chamber of the SMAS device, or they may be situated in more localized regions. As described further below, the locations of instances of particular strands can be identified, and error-correcting procedures can be applied to detection results corresponding to the instances prior to calling the bases to improve the accuracy of the sequencing relative to CLUS devices. Moreover, relative to CLUS devices, for reasonable chemistry failure rates, SMAS devices require fewer instances of each nucleic acid strand to be sequenced to achieve accurate sequencing results.

FIG. 4B illustrates an exemplary plurality of S single-molecule sensors 105, each used by a SMAS device to monitor a respective single-stranded DNA (ssDNA) 101. Each of the plurality of S sensors 105 may be, for example, a magnetic sensor, an optical sensor, etc. FIG. 4B illustrates five single-molecule sensors 105A, 105B, 105C, 105D, and 105E, each of which senses a respective DNA strand 101 (which may be instances of the same DNA strand, or instances of different DNA strands). Each sensor 105 may be, for example, a nanoscale sensor that is so small that only a single DNA strand 101 can bind to the binding site associated with the sensor 105. (For convenience, FIG. 4B shows the strands 101 in contact with the sensors 105, but, as explained further below, in some embodiments, the strands 100 are attached to individual binding sites, each of which is associated with a respective sensor 105.)

Consider clonally amplified DNA bound to a solid surface containing a densely-packed array of sensors 105, as shown in FIG. 4B. The DNA can be replicated either by solid phase amplification (SPA) to create clusters of monoclonal DNA, each strand to be sensed by a different sensor 105, or the DNA can be amplified in bulk and then immobilized on a surface of the SMAS device. If DNA is amplified (e.g., by SPA) on the surface of the fluid chamber of the SMAS device, the sensors 105A, 105B, 105C, 105D, 105E may sense instances of clonal DNA. Alternatively, if the DNA is amplified in bulk off-device and added to the SMAS device's fluid chamber, the amplified DNA strands 101 may be distributed more randomly among the sensors 105.

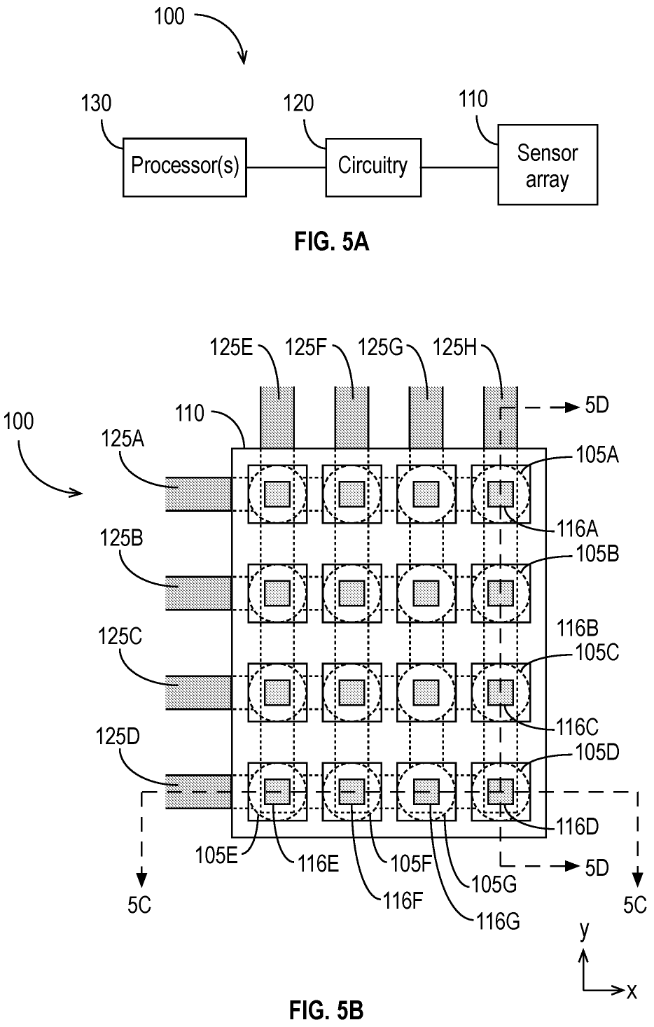

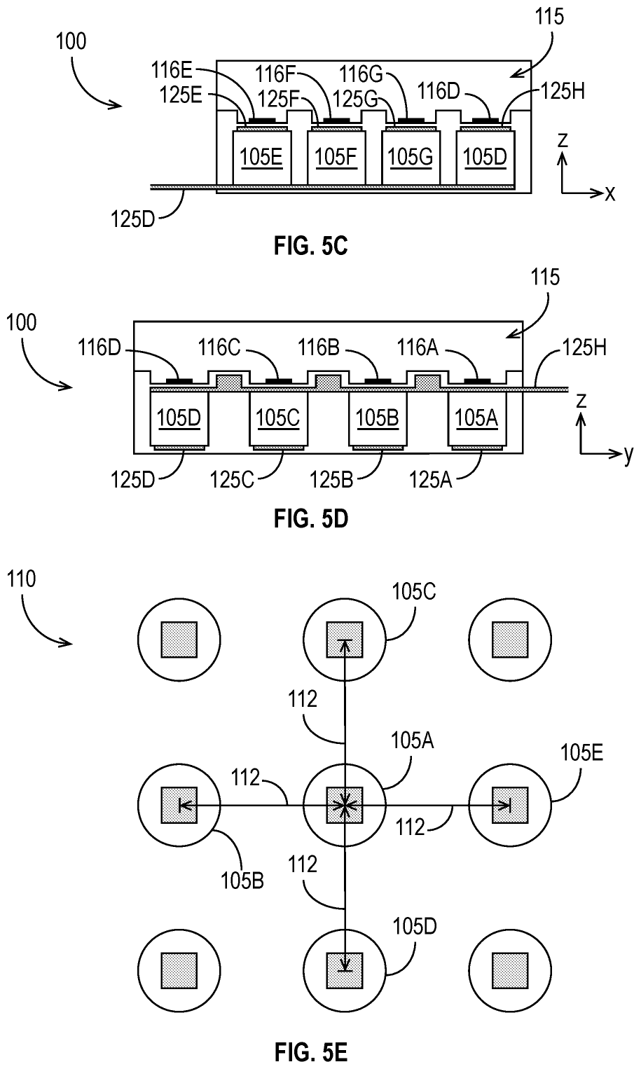

FIG. 5A is a block diagram showing components of an exemplary SMAS device 100 for nucleic acid sequencing in accordance with some embodiments. As illustrated, the device 100 includes a sensor array 110, which is coupled to circuitry 120, which is coupled to at least one processor 130. The sensor array 110 comprises a plurality of sensors 105 (e.g., magnetic, optical, etc.) that may be arranged in any suitable way, as described further below. The characteristics and properties of the sensors 105 in the sensor array 110 are dependent on the type of label used for sequencing.

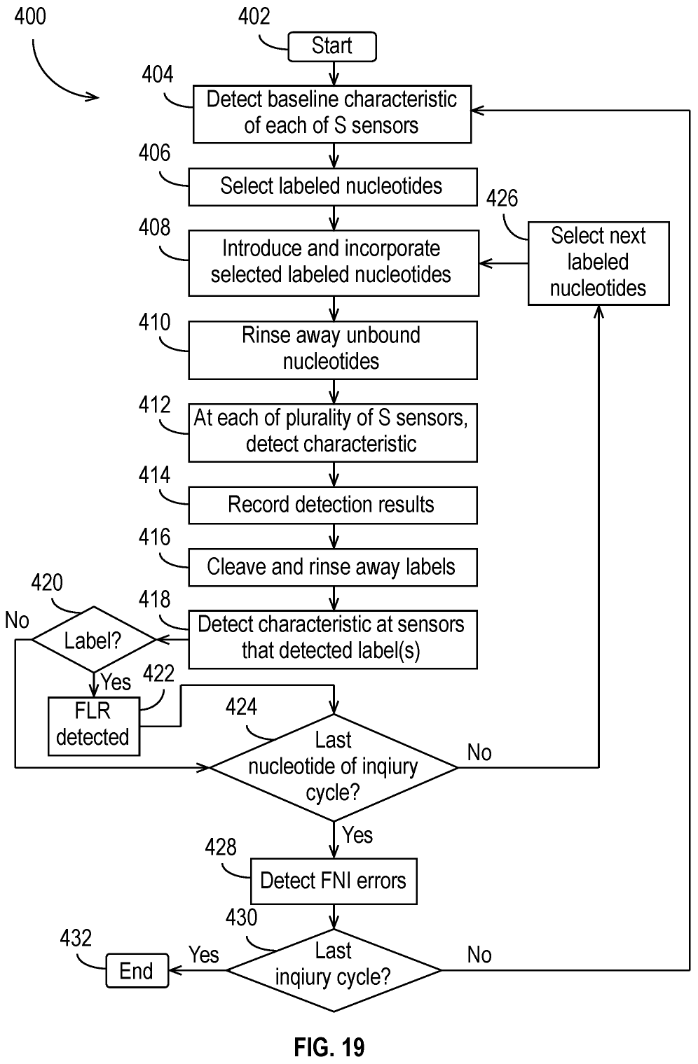

The circuitry 120 can include, for example, one or more lines that allow sensors 105 in the sensor array 110 to be interrogated by the at least one processor 130 (e.g., with the assistance of other components that are well known in the art, such as a current source, etc.). For example, in operation, the processor(s) 130 can cause the circuitry 120 to apply a current to such lines to detect a characteristic of at least one of the plurality of sensors 105 in the sensor array 110, where the characteristic indicates the presence of a label or the absence of any label within range of the sensor 105. In other words, the characteristic (e.g., resistance, frequency, voltage, signal level, etc.) indicates whether a sensor 105 has detected at least one label or has not detected any labels. For example, the at least one processor 130 may assess the value of the characteristic (e.g., a frequency, a wavelength, a magnetic field, a resistance, a noise level, an intensity, a color of light, etc.) and determine that a label was (or was not) detected based on a comparison of the value of the characteristic to a threshold (e.g., by determining whether the value of the characteristic for a sensor 105 meets or exceeds a threshold) or a baseline value. As another example, the at least one processor 130 may compare the obtained characteristic of a sensor 105 to a previously-detected value of the characteristic (e.g., a baseline value for the sensor 105) and to base the determination of whether a label was or was not detected on a change in the value of the characteristic (e.g., a change in magnetic field, resistance, noise level, frequency, wavelength, intensity, color of light, etc.). For example, as described further below in the discussion of FIG. 19, the at least one processor 130 can evaluate the characteristic obtained from a sensor 105 to detect whether a sensor 105 that detected a label during a first inquiry step of a sequencing procedure is still detecting that label following a cleaving step that should have removed the label. Similarly, the at least one processor 130 can evaluate changes in the characteristic from one inquiry step to the next to determine whether a sensor 105 (a) did not detect a label during either inquiry step, (b) detected a label during both inquiry steps, (c) did not detect a label during a first inquiry step but did detect a label during a subsequent inquiry step, and/or (d) did detect a label during a first inquiry step but did not detect a label during a subsequent inquiry step.

The characteristic that is detected depends on the type of label used in the sequencing procedure. The labels may be, for example, fluorescent, in which case the sensors 105 may be optical sensors that can detect, for example, a wavelength, frequency, modulation frequency, color, or intensity of light emitted by the fluorescent labels. Optical sensors suitable for detecting fluorescent labels are well known in the art. In the case that the labels used in the nucleic acid sequencing procedure are fluorescent, in some embodiments, the circuitry 120 allows the at least one processor 130 to detect deviations or fluctuations in the light (or electromagnetic energy) detected by some or all of the sensors 105 in the sensor array 110.

The labels may be, for example, magnetic (e.g., magnetic nanoparticles, organometallic compounds, charged molecules, etc.), in which case the sensors 105 may be magnetic sensors that can detect magnetic characteristics. Magnetic sensors have been described in the applicants' previously-filed patent applications, including, for example, PCT application No. PCT/US20/27290, filed Apr. 8, 2020, entitled “NUCLEIC ACID SEQUENCING BY SYNTHESIS USING MAGNETIC SENSOR ARRAYS” (Attorney Docket No. ROA-1000-WO/P35097-WO), and published on Oct. 15, 2020 as WO 2020/210370. In some embodiments in which the labels are magnetic, the sensors 105 are magnetoresistive (MR) sensors that can detect, for example, a magnetic field or a resistance, a change in magnetic field or a change in resistance, or a noise level. In some embodiments, each of the sensors 105 of the sensor array 110 is a thin film device that uses the MR effect to detect magnetic labels attached to nucleotides incorporated into a single strand of nucleic acid bound to a respective binding site. The sensors 105 may operate as potentiometers with a resistance that varies as the strength and/or direction of the sensed magnetic field changes. In some embodiments using magnetic labels, the sensors 105 comprise a magnetic oscillator (e.g., a spin-torque oscillator (STO)), and the characteristic that indicates whether at least one label is detected is a frequency of a signal associated with or generated by the magnetic oscillator, or a change in the frequency of the signal.

In the case that the labels used in the nucleic acid sequencing procedure are magnetic, in some embodiments, the at least one processor 130, with help from the circuitry 120, detects deviations or fluctuations in the magnetic environment of some or all of the sensors 105 in the sensor array 110. For example, a sensor 105 of the MR type in the absence of a magnetic label should have relatively small noise above a certain frequency as compared to a sensor 105 in the presence of a magnetic label, because the field fluctuations from the magnetic label will cause fluctuations of the moment of the sensing ferromagnet. These fluctuations can be measured using heterodyne detection (e.g., by measuring noise power density) or by directly measuring the voltage of the sensor 105 and evaluated using a comparator circuit to compare to another sensor element that does not sense the binding site. In the case the sensors 105 include STO elements, fluctuating magnetic fields from magnetic labels would cause jumps in phase for the sensors 105 due to instantaneous changes in frequency, which can be detected using a phase detection circuit. Another option is to design the STO such that it oscillates only within a small magnetic field range such that the presence of a magnetic label would turn off the oscillations.

It is to be understood that the examples of labels and sensors 105 provided above are merely exemplary. In general, any type of label that can label nucleotide precursors may be used along with an array 110 of any type of sensor 105 that can detect that type of label.

FIGS. 5B, 5C, and 5D illustrate portions of an exemplary SMAS device 100 for nucleic acid sequencing in accordance with some embodiments. The exemplary SMAS device 100 uses magnetic labels and magnetic sensors 105. FIG. 5B is a top view of the device 100. FIG. 5C is a cross-section view at the position indicated by the long-dash line labeled “5C” in FIG. 5B, and FIG. 5D is a cross-section view at the position indicated by the long-dash line labeled “5D” in FIG. 5B.

The exemplary device 100 shown in FIGS. 5B, 5C, and 5D comprises a sensor array 110 for sensing magnetic labels within a fluid chamber 115. The sensor array 110 includes a plurality of magnetic sensors 105, with sixteen sensors 105 shown in the array 110 of FIG. 5B. It is to be appreciated that an implementation of a SMAS device 100 may include any number of sensors 105 (e.g., hundreds, thousands, or millions of sensors 105). To avoid obscuring the drawing, only seven of the sensors 105 are labeled in FIG. 5B, namely the sensors 105A, 105B, 105C, 105D, 105E, 105F, and 105G. As explained above, the magnetic sensors 105 detect the presence or absence of magnetic labels. In other words, each of the magnetic sensors 105 detects whether there is at least one magnetic label in its vicinity.

Referring now to FIGS. 5C and 5D in conjunction with FIG. 5B, each sensor 105 is illustrated in the exemplary embodiment of the device 100 as having a cylindrical shape. It is to be understood, however, that in general the sensors 105 can have any suitable shape. For example, the sensors 105 may be cuboid in three dimensions. Moreover, different sensors 105 can have different shapes (e.g., some may be cuboid and others cylindrical, etc.). It is to be appreciated that the drawings are merely exemplary.

As shown in FIGS. 5C and 5D, the device 100 includes a fluid chamber 115. The fluid chamber 115 comprises a plurality of binding sites 116 (e.g., S binding sites 116). In some embodiments, the fluid chamber 115 holds fluids (e.g., nucleotide precursors and other fluids) that are used during nucleic acid sequencing procedures. It is to be understood, however, that embodiments in which the fluid chamber 115 does not hold fluids are contemplated and are within the scope of the disclosures herein. For example, the binding sites 116 may be disposed on a removable (or movable) part (e.g., a panel, plate, slide, etc.), which may be dipped into reagents and other fluids after nucleic acid strands have been attached to the binding sites 116 and then situated so that the sensors 105 can detect labels. Thus, although the name of the fluid chamber 115 suggests that it holds fluids, it is not a requirement that the fluid chamber 115 hold fluids.

As shown in FIGS. 5B, 5C, and 5D, each of the sensors 105 is associated with a respective binding site 116. (For simplicity, this document refers generally to the binding sites by the reference number 116. Individual binding sites are given the reference number 116 followed by a letter.) In other words, the sensors 105 and the binding sites 116 are in a one-to-one relationship. As shown in FIG. 5B, the sensor 105A is associated with the binding site 116A, the sensor 105B is associated with the binding site 116B, the sensor 105C is associated with the binding site 116C, the sensor 105D is associated with the binding site 116D, the sensor 105E is associated with the binding site 116E, the sensor 105F is associated with the binding site 116F, and the sensor 105G is associated with the binding site 116G. Each of the other, unlabeled sensors 105 shown in FIG. 5B is also associated with a respective binding site 116. In the example embodiment of FIGS. 5B, 5C, and 5D, each sensor 105 is shown disposed below its respective binding site 116, but it is to be appreciated that the binding sites 116 may be in other locations relative to their respective sensors 105. For example, the binding sites 116 may be to the sides of their respective sensors 105.

Each of the binding sites 116 is configured to bind no more than one strand of nucleic acid (e.g., ssDNA) to the SMAS device 100 within the fluid chamber 115. In other words, each binding site 116 has characteristics and/or features that allow one, and only one, strand of nucleic acid to be bound to it for sensing by a respective sensor 105 (and for sequencing). The respective sensor 105 can thereafter detect labels attached to nucleotides incorporated into the strand of nucleic acid bound to the binding site 116 during a nucleic acid sequencing procedure, as discussed further below. In some embodiments, the binding site 116 has a structure (or multiple structures) configured to anchor nucleic acid to the binding site 116. For example, the structure (or structures) may include a cavity or a ridge. FIGS. 5C and 5D illustrate the binding sites 116 as extending from the surface of the fluid chamber 115, but it is to be recognized that the binding sites 116 may be flush with or etched into the surface of the fluid chamber 115.

The binding sites 116 can have any suitable size and shape that facilitates the attachment of one, and only one, strand of nucleic acid to each binding site 116. For example, the shapes of the binding sites can be similar or identical to the shapes of the sensors 105 (e.g., if the sensors 105 are cylindrical in three dimensions, the binding sites 116 can also be cylindrical, either protruding from the surface of the fluid chamber 115 or forming a fluid container within the surface of the fluid chamber 115, with a radius that can be larger, smaller, or the same size as the radius of the respective sensor 105; if the sensors 105 are cuboid in three dimensions, the binding sites 116 can also be cuboid with a surface 116 that is larger, smaller, or the same size as the closest part of the sensors 105, etc.). In general, the binding sites 116 and the surface of the fluid chamber 115 can have any shapes and characteristics that facilitate the attachment of a single nucleic acid strand to each binding site 116 and allow the sensors 105 to detect labels attached to incorporated nucleotides at their respective binding sites 116.

FIGS. 5C and 5D illustrate an enclosed fluid chamber 115 with a top portion that extends in the x-y plane, but there is no requirement for the fluid chamber 115 to be enclosed. In some embodiments, the surface of the fluid chamber 115 has properties and characteristics that protect the sensors 105 from whatever fluids are in the fluid chamber 115, while still allowing the nucleic acid strands to bind to the binding sites 116 and the sensors 105 to detect labels that are attached to nucleotides incorporated in nucleic acid strands attached to the binding sites 116. The material of the fluid chamber 115 (and possibly of the binding sites 116) may be or comprise an insulator. In some embodiments, the surface of the fluid chamber 115 comprises an organic polymer, a metal, or a silicate. The fluid chamber 115 may include, for example, a metal oxide, silicon dioxide, polypropylene, gold, glass, or silicon. The thickness of the surface of the fluid chamber 115 may be selected so that the sensors 105 can detect magnetic labels attached to nucleotides incorporated into nucleic acid strands bound to the binding sites 116 within the fluid chamber 115. In some embodiments, the surface is approximately 3 to 20 nm thick so that each sensor 105 is between approximately 5 nm and approximately 50 nm from any label attached to a nucleotide incorporated into a nucleic acid strand bound to the sensor 105's respective binding site 116. It is to be understood that these values are merely exemplary. It will be appreciated that an implementation may have a fluid chamber 115 with a thicker or thinner surface.

The circuitry 120 of the device 100 may include one or more lines 125. In some embodiments, each of the plurality of sensors 105 is coupled to at least one line 125. In the example shown in FIGS. 5B, 5C, and 5D, the device 100 includes eight lines 125A, 125B, 125C, 125D, 125E, 125F, 125G, and 125H. (For simplicity, this document refers generally to the lines by the reference number 125. Individual lines are given the reference number 125 followed by a letter.) Pairs of lines 125 can be used to access (e.g., interrogate) individual sensors 105. In the exemplary embodiment shown in FIGS. 5B, 5C, and 5D, each sensor 105 of the sensor array 110 is coupled to two lines 125. For example, the sensor 105A is coupled to the lines 125A and 125H; the sensor 105B is coupled to the lines 125B and 125H; the sensor 105C is coupled to the lines 125C and 125H; the sensor 105D is coupled to the lines 125D and 125H; the sensor 105E is coupled to the lines 125D and 125E; the sensor 105F is coupled to the lines 125D and 125F; and the sensor 105G is coupled to the lines 125D and 125G. In the exemplary embodiment of FIGS. 5B, 5C, and 5D, the lines 125A, 125B, 125C, and 125D are shown residing under the magnetic sensors 105, and the lines 125E, 125F, 125G, and 125H are shown residing above the magnetic sensors 105. FIG. 5C shows the sensor 105E in relation to the lines 125D and 125E, the sensor 105F in relation to the lines 125D and 125F, the sensor 105G in relation to the lines 125D and 125G, and the sensor 105D in relation to the lines 125D and 125H. FIG. 5D shows the sensor 105D in relation to the lines 125D and 125H, the sensor 105C in relation to the lines 125C and 125H, the sensor 105B in relation to the lines 125B and 125H, and the sensor 105A in relation to the lines 125A and 125H.

The sensors 105 of the exemplary SMAS device 100 of FIGS. 5B, 5C, and 5D are arranged in a rectangular pattern sensor array 110. (It is to be appreciated that a square pattern is a special case of a rectangular pattern.) Each of the lines 125 identifies a row or a column of the sensor array 110. For example, each of the lines 125A, 125B, 125C, and 125D identifies a different row of the sensor array 110, and each of the lines 125E, 125F, 125G, and 125H identifies a different column of the sensor array 110. As shown in FIG. 5C, each of the lines 125E, 125F, 125G, and 125H is in contact with one of the sensors 105 along the cross-section (namely, line 125E is in contact with the top of sensor 105E, line 125F is in contact with the top of sensor 105F, line 125G is in contact with the top of sensor 105G, and line 125H is in contact with the top of sensor 105D), and the line 125D is in contact with the bottom of each of the sensors 105E, 105F, 105G, and 105D. Similarly, and as shown in FIG. 5D, each of the lines 125A, 125B, 125C, and 125D is in contact with the bottom of one of the sensors 105 along the cross-section (namely, line 125A is in contact with the bottom of sensor 105A, line 125B is in contact with the bottom of sensor 105B, line 125C is in contact with the bottom of sensor 105C, and line 125D is in contact with the bottom of sensor 105D), and the line 125H is in contact with the top of each of the sensors 105D, 105C, 105B, and 105A.

The sensors 105 and portions of the lines 125 connecting to the sensor array 110 are illustrated in FIG. 5B using dashed lines to indicate that they may be embedded within the device 100. As explained above, the sensors 105 may be protected (e.g., by an insulator) from the contents of the fluid chamber 115, which itself might be enclosed. Accordingly, it is to be understood that the various illustrated components (e.g., lines 125, sensors 105, binding sites 116, etc.) are not necessarily visible in a physical instantiation of the device 100 (e.g., they may be embedded in or covered by protective material, such as an insulator).

In some embodiments, some or all of the binding sites 116 reside in nanowells or trenches in lines 125 passing over the sensors 105. For example, as shown in the example of FIG. 5D, the line 125H may be thinner over the sensors 105 than it is between the sensors 105. For example the line 125H has a first thickness above the sensor 105D, a second, larger thickness between the sensors 105D and 105C, and the first thickness above the sensor 105C. Such a configuration may be advantageously fabricated using conventional thin-film fabrication methods (e.g., by depositing material, applying a mask to the deposited material, and removing (e.g., by etching) some of the deposited material in accordance with the mask). Both the binding sites 116 and, if present, nanowells may be fabricated using conventional techniques.

To simplify the explanation, FIGS. 5B, 5C, and 5D illustrate an exemplary device 100 with only sixteen sensors 105 in the sensor array 110, only sixteen corresponding binding sites 116, and eight lines 125. It is to be appreciated that the device 100 may have fewer or many more sensors 105 in the sensor array 110, and, accordingly, it may have more or fewer binding sites 116. Similarly, embodiments that include lines 125 may have more or fewer lines 125. In general, any configuration of sensors 105 and binding sites 116 that allows the sensors 105 to detect labels attached to nucleotides incorporated into single nucleic acid strands attached to the binding sites 116 may be used. Similarly, any configuration of one or more lines 125 or some other mechanism that allows the determination of whether the sensors 105 have sensed one or more labels may be used. The examples presented herein are not intended to be limiting.

As explained above, the sensors 105 shown in FIGS. 5B, 5C, and 5D may be magnetic sensors 105. Accordingly, the sensors 105 are in close proximity to the binding sites 116 and, therefore, they are also in close proximity to the nucleic acid strands that are bound to the binding sites 116. It is to be understood that the appropriate location for the sensor array 110 in relation to the binding sites 116 depends in part on the type of label being used and, therefore, the type of sensor 105 being used. For example, if the labels are fluorophores, and the sensors 105 are optical sensors, it may be appropriate for the sensor array 110 to be remote from the binding sites 116 (e.g., situated above the binding sites 116).

Although FIGS. 5B, 5C, and 5D (and other drawings herein) illustrate sensors 105 and binding sites 116 in a one-to-one relationship, it is to be appreciated that each binding site 116 can be sensed by more than one sensor 105. The characteristic that distinguishes a SMAS device 100 from a CLUS device is that no sensor 105 of a SMAS device 100 senses more than one nucleic acid strand instance. If a SMAS device 100 has more sensors 105 than binding sites 116, it may be possible for at least some nucleic acid strand(s) to be sensed by multiple sensors 105 (e.g., to improve the accuracy of label detection).

The exemplary sensor array 110 shown and described in the context of FIGS. 5B, 5C, and 5D is a rectangular array, with the sensors 105 arranged in rows and columns. In other words, the plurality of sensors 105 of the sensor array 110 is arranged in a rectangular grid pattern. In some embodiments, adjacent rows and columns of the rectangular grid pattern are equidistant from each other, which results in the sensors 105 being arranged in a square grid (or lattice) pattern as illustrated in FIG. 5E. In embodiments in which the sensors 105 are arranged in a square grid pattern, each sensor 105 has up to four nearest neighbors. For example, as shown in FIG. 5E, the sensor 105A has the four nearest neighbors labeled as 105B, 105C, 105D, and 105E. The closest sensors 105 are a nearest-neighbor distance 112 away, as shown in FIG. 5E. Thus, each of the sensors 105B, 105C, 105D, and 105E is a distance 112 away from the sensor 105A.

A commercially viable SMAS device 100 may use high-precision nanoscale fabrication of densely-packed nanoscale sensors 105 capable of recognizing individual labels. The sizes of the functionalized binding sites 116 can be similar to the size of, for example, DNA with a label attached so that multiple strands cannot bind to the same binding site 116 or be sensed by the same sensor 105. A good established metric for evaluating sequencer's commercial competitiveness is how densely DNA strands can be packed together in the fluid chamber 115.

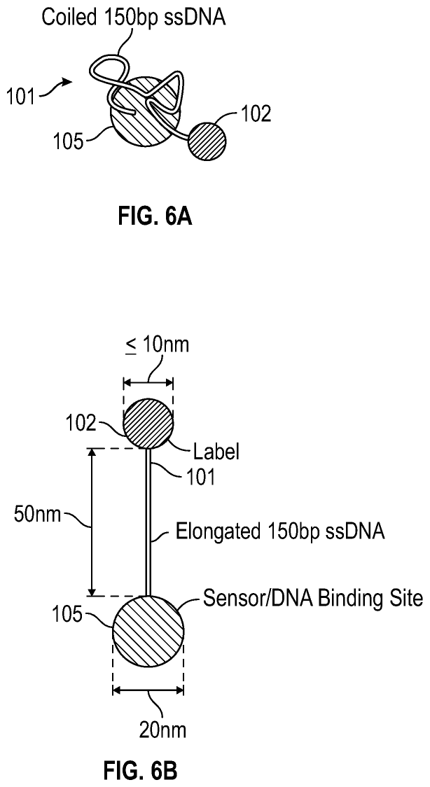

The appropriate value of the nearest-neighbor distance 112, which may then be used to determine the size of the SMAS device 100 and/or the maximum number of sensors 105 that can fit within a SMAS device 100 of a selected size, can be determined based on the properties of the sensors 105, the lengths of nucleic acid strands the device 100 is intended to sequence, and the properties of the labels being used. For example, the combined length of the nucleic acid strands and the size of the label to be used can provide a physical limitation on how closely two sensors 105 in a SMAS device 100 can be positioned. In some embodiments, the size of the sensors 105 may be limited by the nanoscale patterning capabilities of a process used to manufacture the SMAS device 100. For example, using technology available at the time of writing, the size of each magnetic sensor 105 (e.g., assuming cylindrical sensors 105, the diameter of the sensors 105 in the x-y plane) may be around 20 nm. Assuming the type of nucleic acid to be sequenced is DNA, and it is desirable to sequence fragments up to 150 base pairs (bp) in length, the maximum length of a DNA strand 101 to be sequenced is approximately 50 nm in the elongated state, although ssDNA conformation can vary between elongated and coiled, as shown in FIG. 6A, depending on the ionic strength of the buffer. Because the label 102 participates in single-molecule reactions, the label 102 should have molecular dimensions. For a SMAS device 100 using magnetic sensors 105, the labels 102 can be, for example, superparamagnetic nanoparticles, organometallic compounds, or any other functional molecular group that can be detected by nanoscale magnetic sensors 105. Thus, it is assumed that each label 102 has a size that is no more than about 10 nm. With these assumptions, FIG. 6B shows the relative dimensions of the magnetic sensor 105, the DNA strand 101 in its elongated state, and the magnetic label 102.

A practical SMAS device 100 that uses magnetic sensors 105 to detect magnetic nanoparticles used as labels 102 can be implemented using existing technologies. For the sake of the argument, it is assumed that only the labels 102 within 20 nm of edge of a sensor 105 are detected. The detection range of each sensor 105 is small because the magnetic labels 102 that may be selected for nucleic acid sequencing applications (e.g., superparamagnetic nanoparticles, organometallic compounds, etc.) do not generate significant perturbations to the detected magnetic field. Although a label 102 attached to a nucleotide incorporated into a ssDNA bound to a particular sensor 105's binding site 116 can reside temporarily outside of the range of the respective sensor 105, as ssDNA assumes various conformation states during the detection process, it is desirable that labels not be permitted to reach the sensitive spaces (detection regions) of neighboring sensors 105 when the ssDNA assumes its fully elongated state.

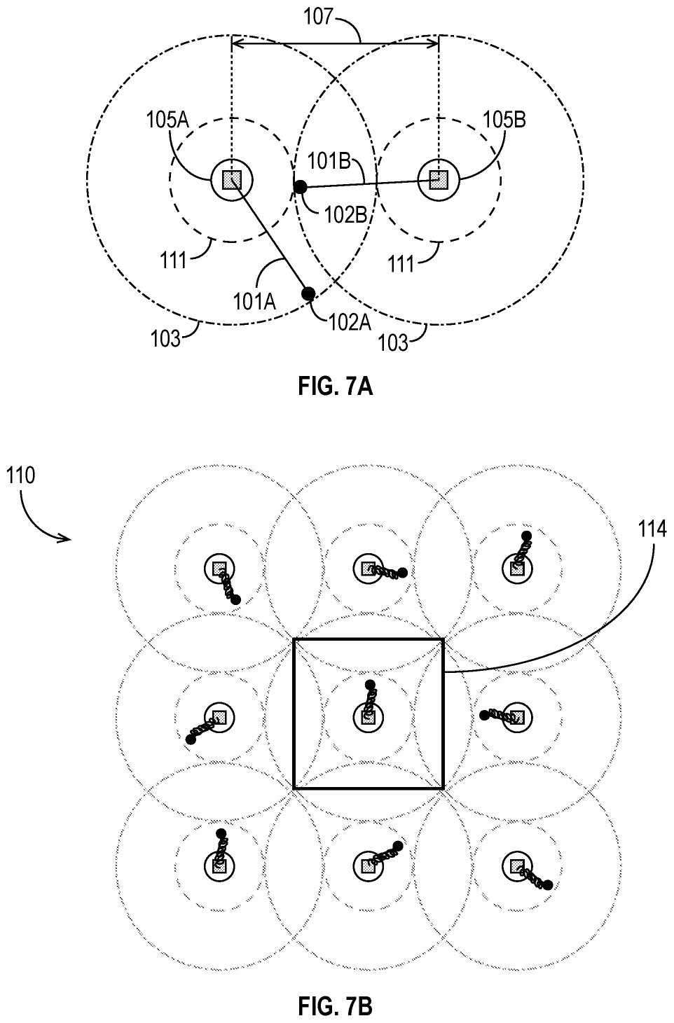

The sensor-packing limit for a practical SMAS device 100 can be derived, for example, assuming the labels are superparamagnetic nanoparticles (e.g., iron oxide, iron platinum, etc.), and the sensor array 110 of the SMAS device 100 is a rectangular (e.g., square) array of magnetic tunnel junctions (MTJs) similar to those used in non-volatile data storage applications. In this case, the area of each nanoscale sensor 105 or its immediate proximity can be functionalized to serve as a respective binding site 116. A simple geometrical arrangement for estimating the sensor-array packing limit of a SMAS device 100 is shown in FIG. 7A, which shows two sensors 105A, 105B. Each sensor 105A, 105B, assumed solely for convenience to have a cylindrical shape, is assumed to have a diameter of about 20 nm (as explained above) and is assumed to be able to detect any label within 20 nm from its edge. The sensing area boundaries 111 are denoted by the inner dashed lines shown in FIG. 7A. The sensor 105A senses the DNA strand 101A bound to its binding site, and the sensor 105B senses the DNA strand 101B bound to its binding site. The maximum reaches (e.g., when the DNA strands with 150 bases are in their fully uncoiled states) of the labels 102A, 102B, when attached to nucleotides incorporated into the strands 101A, 101B, are shown by the outer dash-dot circles 103. For sequencing results to be accurate, it is desirable for each sensor 105 to detect only labels 102 attached to nucleotides incorporated into the DNA strand 101 bound to the sensor 105's respective binding site 116. Thus, with the assumptions described above, the minimum nearest-neighbor distance 112 between sensors 105 to avoid cross-talk (e.g., detecting labels 102 attached to nucleotides incorporated into a nucleic acid strand 101 bound to another sensor 105's binding site 116) is approximately 100 nm.

In some embodiments of the SMAS device 100, sensors 105 (e.g., MTJs) are arranged in a square lattice that is compatible with existing cross-point MRAM sensor geometries, as shown in FIG. 7B. The area of the unit cell 114 is 10⁴ nm², which allows each DNA strand 101 to extend throughout an area of approximately 10⁴ nm², which yields a DNA surface density for the SMAS device 100 of approximately 10¹⁰ strands/cm². Assuming the use of at least ten instances of each strand 101 in the sensor array 110, approximately 10⁹ unique strands/cm² can be sequenced simultaneously, generating 150 Gbase (1 billion×150 bp DNA strand length) of information per square centimeter of the sensor array 110. In the ideal case (e.g., when the chemistry failure rate is low only three DNA instances are needed, as discussed further below), approximately 3.3×10⁹ different strands/cm² can be sequenced simultaneously, and approximately 500 Gbase of data can be generated per square centimeter of the sensor array 110.

As a specific example, a SMAS device 100 having a configuration similar to the single Toshiba 4 Gbit density STT-MRAM chip first introduced at the International Electron Devices Meeting (IEDM) in 2016 could potentially generate approximately 600 Gbase of high-quality data. The minimum distance 112 between sensors 105 of the Toshiba platform is 90 nm, which is only slightly below the estimated minimum distance 112 of 100 nm derived above. Accordingly, the cross-talk using a configuration similar to the Toshiba platform would likely be low even with 150 base-length ssDNA, but shorter fragments could be sequenced to reduce cross-talk even further.

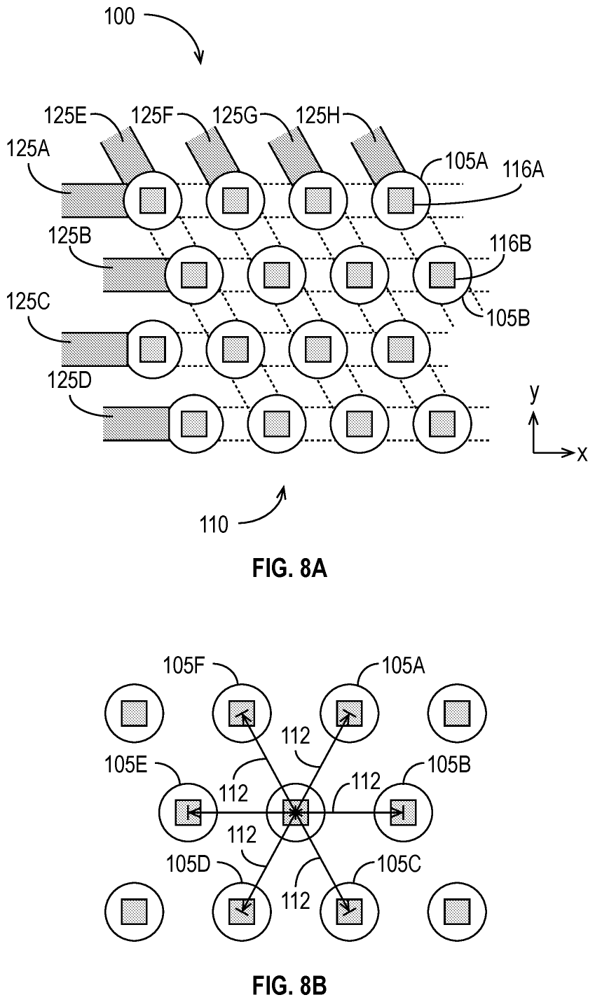

It is to be understood that the arrangement of sensors 105 in a grid pattern (e.g., a square lattice as shown in FIG. 7B) is one of many possible arrangements. It will be appreciated by those having ordinary skill in the art that other arrangements of the sensors 105 are possible and are within the scope of the disclosures herein. For example, the sensors 105 may be arranged in a hexagonal pattern, as shown in FIG. 8A, which shows a top view of the SMAS device 100. The exemplary SMAS device 100 shown in FIG. 8A comprises a sensor array 110 for sensing labels 102 within a fluid chamber 115. The sensor array 110 includes a plurality of sensors 105, with sixteen sensors 105 shown. It is to be appreciated that an implementation of the device 100 may include any number of sensors 105 (e.g., hundreds, thousands, millions etc.). To avoid obscuring the drawing, only two of the sensors 105 are labeled in FIG. 8A, namely the sensors 105A and 105B. As explained above, the sensors 105 may be, for example, magnetic sensors (e.g., to detect magnetism or the effects of magnetic nanoparticles). As explained above at least in the discussion of FIGS. 5B, 5C, and 5D, in general the sensors 105 can have any suitable size and shape.

As shown in FIG. 8A, each of the sensors 105 is associated with a respective binding site 116. In other words, the sensors 105 and the binding sites 116 are in a one-to-one relationship. As shown in FIG. 8A, the sensor 105A is associated with the binding site 116A, the sensor 105B is associated with the binding site 116B, and each of the other, unlabeled sensors 105 is also associated with a respective binding site 116. In the example embodiment of FIG. 8A, each sensor 105 is shown disposed below its respective binding site 116, but it is to be appreciated that the binding sites 116 may be in other locations relative to their respective sensors 105. For example, the binding sites 116 may be to the sides of their respective sensors 105. The discussion of the binding sites 116 in the explanations of at least FIGS. 5B, and 5D applies to FIG. 8A and other figures showing binding sites 116 and is not repeated here.

The exemplary SMAS device 100 of FIG. 8A also includes a fluid chamber 115, described above in the discussion of FIGS. 5B, 5C, and 5D. Those descriptions also apply to FIG. 8A and are not repeated here.

The circuitry 120 of the device 100 of FIG. 8A may include one or more lines 125. Each of the lines 125 in the exemplary embodiment of FIG. 8A identifies a row or a diagonal column of the sensor array 110. For example, each of the lines 125A, 125B, 125C, and 125D identifies a different row of the sensor array 110, and each of the lines 125E, 125F, 125G, and 125H identifies a different diagonal column of the sensor array 110. In the example shown in FIG. 8A, the device 100 has eight lines 125A, 125B, 125C, 125D, 125E, 125F, 125G, and 125H, and pairs of lines 125 can be used to access individual sensors 105. For example, the lines 125A and 125H can be used to access the sensor 105A, and the lines 125B and 125H can be used to access the sensor 105B. The lines 125 may be oriented under and/or over the sensors 105 as described in the discussion of FIGS. 5B, 5C, and 5D, among others.

Although FIG. 8A illustrates an exemplary device 100 with only sixteen sensors 105 in the sensor array 110, only sixteen corresponding binding sites 116, and eight lines 125, it is to be appreciated that the SMAS device 100 may have fewer or many more sensors 105 in the sensor array 110, and, accordingly, it may have more or fewer binding sites 116. Furthermore, an SMAS device 100 may have more or fewer lines 125. In general, any configuration of sensors 105 and binding sites 116 that allows the sensors 105 to detect labels attached to nucleotides incorporated into single nucleic acid strands attached to the binding sites 116 may be used. Similarly, any configuration of one or more lines 125 or some other mechanism that allows the determination of whether the sensors 105 have sensed one or more labels may be used.

As illustrated in FIG. 8B, when the sensors 105 are arranged in a hexagonal pattern, each sensor 105 has up to six nearest neighbors, all at a nearest-neighbor distance 112. In other words, each sensor 105 is a nearest-neighbor distance 112 away from each of the six other sensors 105 that are closest to it. For example, as shown in FIG. 8B, the unlabeled sensor 105 in the middle of the drawing has six nearest neighbor sensors 105, labeled as 105A, 105B, 105C, 105D, 105E, and 105F, all of which are a nearest-neighbor distance 112 away.

The binding site 116 packing limit for SMAS devices 100 that use optical sensors and fluorescent labels 102 (e.g., fluorophores) with a hexagonal pattern of binding sites 116 can be derived. Assuming the labels 102 are fluorophores, the binding sites 116 are in a hexagonal pattern, and the sensor array 110 is remote from the binding sites 116, single-molecule fluorescence from the labels 102 may be projected into the far-field where it may be detected by a sensor array 110 comprising photo-sensitive sensors 105. Single-molecule super-resolution imaging techniques, such as those described in C. G. Galbraith and J. A. Galbraith, “Super-resolution microscopy at a glance,” Journal of Cell Science, Vol. 124(10), 1607-11 (2011), can be employed to resolve the positions within the SMAS device 100 of individual fluorophore labels 102. The positions of the fluorophore labels 102 can be resolved because the DNA packing dimensions are far below the diffraction limit. Although this type of detection can be somewhat complex and/or expensive, the technique has been recently introduced in commercial sequencing systems to improve the throughput of cluster-based sequencers. Moreover, this technique may be implemented in imaging of large single-molecule arrays in the near future.

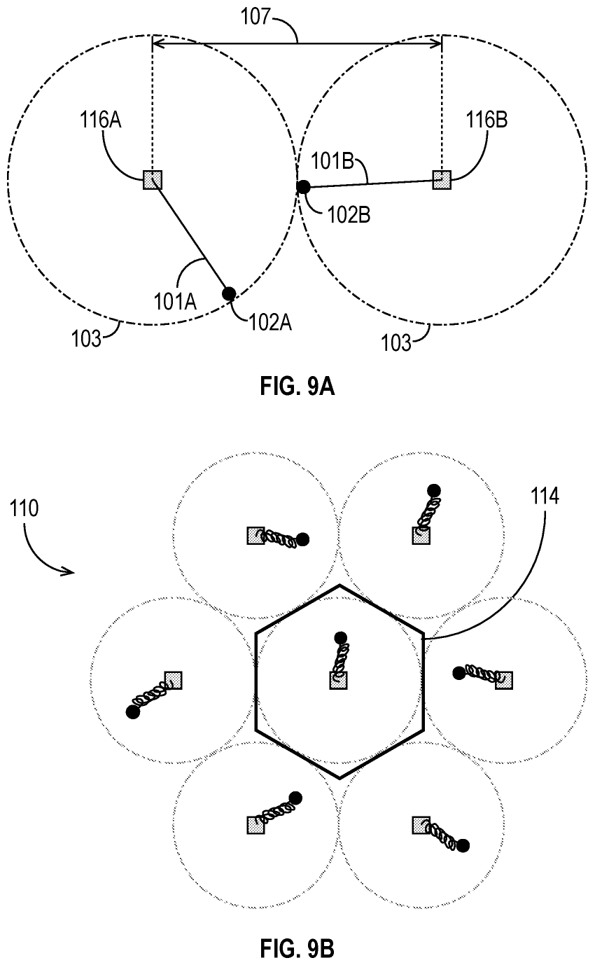

A simple geometrical arrangement for estimating the packing limit for binding sites 116 situated in a hexagonal pattern in a SMAS device 100 that uses fluorophore labels 102 is shown in FIG. 9A. The DNA strand 101A is bound to the binding site 116A, and the DNA strand 101B is bound to the binding site 116B. (The sensors 105 are not illustrated in FIG. 9A because it is assumed that the sensor array 110 is remote from the binding sites.) The maximum reaches (e.g., when the DNA strand with 150 bases is in its fully uncoiled state) of the labels 102A, 102B, when attached to incorporated nucleotides, are shown by the dash-dot circles 103. To avoid cross-talk, fluorophore labels 102 attached to neighboring binding sites 116 are not permitted to occupy overlapping spaces during the imaging process, e.g., a fluorophore label 102A attached to a particular binding site 116A should not be allowed to reach the space accessible to a fluorophore label 102B attached to a neighboring binding site 116B as the ssDNA 101A explores its allowed conformation states. This restriction also helps avoid fluorescence quenching. Assuming the use of fluorophore labels 102, the binding sites 116 can be packed densely in a hexagonal lattice as shown in FIG. 9B. Assuming that the maximum length of a 150 bp DNA strand 101 is 50 nm, the size of the fluorophore labels 102 is 10 nm, the minimum distance from the center of each binding site 116 to its edge is 20 nm, and each DNA strand 101 binds to the center of its respective binding site 116, the minimum distance 112 is 140 nm. Thus, as shown in FIG. 9B, every DNA strand 101 is allowed to occupy a unit cell 114 that has an area of 1.7×10⁴ nm², which yields a DNA surface density of 5.9×10⁹ strands/cm², or 5.9×10⁸ unique strands/cm² if approximately 10 instances of each DNA strand are present in the SMAS device 100. The SMAS device 100 would generate about 90 Gbase of data from every square centimeter of the sensor array 110. In the best case scenario when only 3 DNA replicates are needed, the sensor array 110 holds approximately 2×10⁹ unique DNA strands/cm², and the SMAS device 100 is able to generate approximately 300 Gb of data from every square centimeter of the sensor array 110.

The discussion of the hexagonal array above was in the context of fluorophore labels 102 and optical sensors 105. It is also possible to use a hexagonal arrangement of magnetic sensors 105. The sensor-packing limit for a SMAS device 100 with a hexagonal arrangement of binding sites 116 and magnetic sensors 105 can be derived as described above in the discussion of FIGS. 7A and 7B. For magnetic sensors 105, the nearest neighbor distance 112 is approximately 100 nm, which means the (hexagonal) unit cell area 114 (see FIG. 9B) is approximately 8.7×10 3 nm².

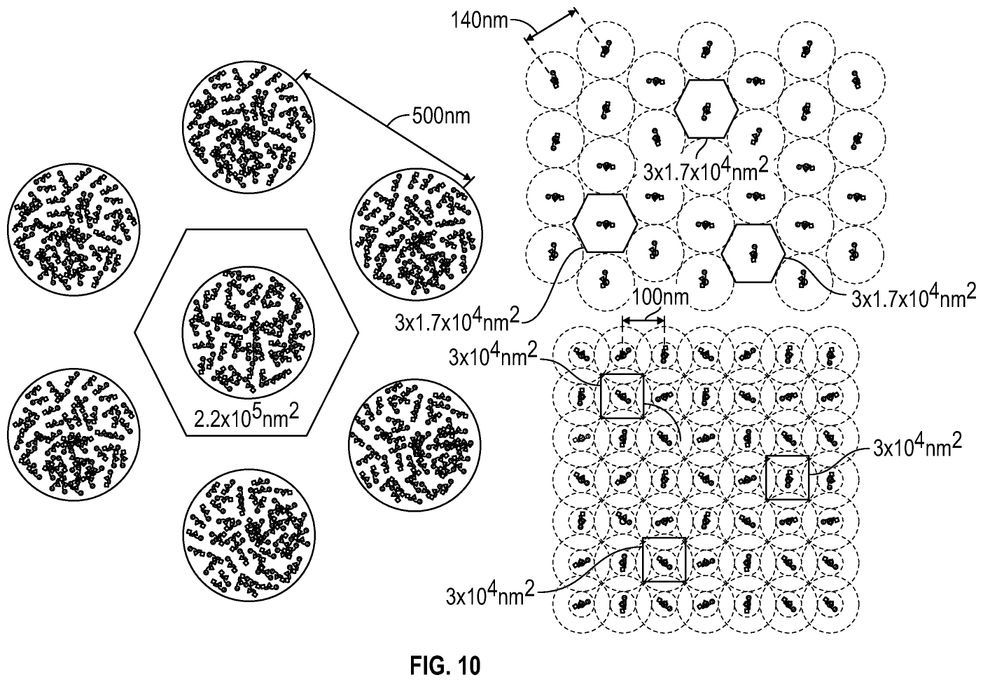

FIG. 10 compares the densities of the SMAS implementations described in the context of FIGS. 7A and 7B (magnetic labels 102 and magnetic sensors 105) and FIGS. 9A and 9B (fluorescent labels 102 and optical sensors 105) to that of current state-of-the-art CLUS sequencers. For the sake of the argument, it is assumed that the pitch of the nanowell array of the patterned flow-cells is approximately 500 nm. As shown in the left-hand panel of FIG. 10, the nanowells of the CLUS sequencer are arranged in a hexagonal lattice with a 500 nm lattice constant. Each nanowell holds between about 50 and about 200 identical DNA strands (e.g., produced by solid phase bridge amplification). The upper right-hand side of FIG. 10 shows a hexagonal SMAS lattice using fluorophore labels and super-resolution imaging (e.g., as described in the context of FIGS. 9A and 9B), and the lower right-hand side of FIG. 10 shows a square SMAS lattice using superparamagnetic nanoparticle labels and a sensor array 110 of MTJs (e.g., as described in the context of FIGS. 7A and 7B). The three representations in FIG. 10 are scaled proportionally to show how the SMAS lattice configurations compare to the CLUS configuration. The black hexagons (left and upper-right) and squares (lower-right) mark the unit cells holding the minimum number of individual molecules needed to call the sequence of a nucleic acid strand. The ideal case, in which only three DNA strands are needed for successful base calling, discussed in further detail below, is illustrated for the SMAS lattices. It is to be noted that in the SMAS cases (right-hand side of FIG. 10), DNA instances are distributed randomly throughout the sensor array 110, and their positions can be identified during the first sequencing cycles, as discussed further below.

As shown in FIG. 10, the area of the unit cells of the CLUS device is 2.2×10⁵ nm², which corresponds to a DNA cluster density of 4.6×10⁸ clusters/cm². With the assumptions made above, the CLUS sequencer generates approximately 70 Gbase of data for every square centimeter of the sensing area. In contrast, in the ideal case when only three instances of a strand are used, the SMAS devices 100 generate approximately 500 Gb/cm² (magnetic sensors 105 (e.g., MTJs) and magnetic labels 102 (e.g., superparamagnetic nanoparticles)) and approximately 300 Gb/cm² (optical sensors 105 (super-resolution imaging) and fluorescent labels 102) of data. The results for the CLUS sequencer and the exemplary implementations of the SMAS device 100 are summarized in the following table, which estimates sequencing throughput assuming only three instances of each DNA strand and assuming ten instances of each DNA strand for the SMAS implementation.

| | Cluster/DNA | Estimated | Estimated |
|---|---|---|---|
| | Strand | Throughput | Throughput |
| | Separation | (Gb/cm²) | (Gb/cm²) |
| Platform | [nm] | (3 DNA instances) | (10 DNA instances) |
| CLUS | ~500 | ~70 | ~70 |
| Fluorescence | 140 | ~300 | ~90 |
| SMAS | | | |
| Magnetic | 100 | ~500 | ~150 |
| SMAS | | | |

The table above shows that the SMAS device 100 outperforms the state-of-the-art CLUS device when the number of DNA instances used for algorithmic error correction, described further below, is small (e.g., <10). As the error-correction procedure relies on more instances of each ssDNA, the SMAS device 100 starts behaving like a CLUS device, and there is little to no benefit in sensing individual molecules rather than clusters. Fluorescence SMAS essentially represents the limit of reducing the cluster to a single molecule. One approach to reduce sequencing cost is to shrink the cluster sizes and pack DNA clusters closer to each other in order to obtain more information from a fixed sensing area. Although this approach reduces the amount of reagents needed to run sequencing chemistry, it also significantly increases the complexity and the cost of the imaging hardware by constantly pushing the limits of what is currently possible in commercial optical instruments. The strategy is an uphill struggle because the in-scaling cannot be done without parallel improvements in chemistry. This is because as the clusters get smaller every reaction matters more, and chemistry failures happening stochastically on a single molecule-level become more vocal and less tolerated.

The cost of implementing super-resolution imaging in CLUS devices is what makes SMAS devices 100, and particularly SMAS devices 100 that use magnetic sensors 105 and magnetic labels, a possibly disruptive sequencing alternative. The SMAS devices 100 disclosed here, and particularly those that use magnetic sensors 105, promise superior throughput at a significantly lower instrument cost by leveraging technologies and high-volume manufacturing developed by massive semiconductor and data storage industries.

### SMAS Sequencing Protocols

As explained above, when SMAS devices 100 are used for nucleic acid sequencing, nucleic acid strands may be amplified either before the nucleic acid is added to the SMAS device 100 or afterward (e.g., using bridge amplification). Regardless of how the nucleic acid is amplified, the strands can be sequenced by SBS (e.g., by synthesizing dsDNA from ssDNA) one base at a time. The SMAS sequencing protocols are described assuming the nucleic acid being sequenced is DNA. It is to be understood that the disclosed protocols can be modified for sequencing of other nucleic acids. With an understanding of the disclosures herein, such modifications will be within the ability of a person having ordinary skill in the art.

To simplify the analysis and illustrate the benefits of using the disclosed SMAS devices 100 rather than CLUS sequencers, consider DNA sequencing protocols in which a single type of a label (e.g., molecular, fluorescent, magnetic, etc.) is attached to all four nucleotides (A, T, C, and G). In other words, identical labels of some type are attached to each of the four nucleotides (e.g., if the selected label 102 is a particle of FePt, then each of A, T, C, and G is labeled with FePt particles). These labeled nucleotides are then incorporated into a DNA strand one base at a time using termination chemistry, e.g., once a nucleotide is incorporated, the label 102 is cleaved before polymerase moves on to the next base. The sensors 105 detect the labels 102 attached to the nucleotides.

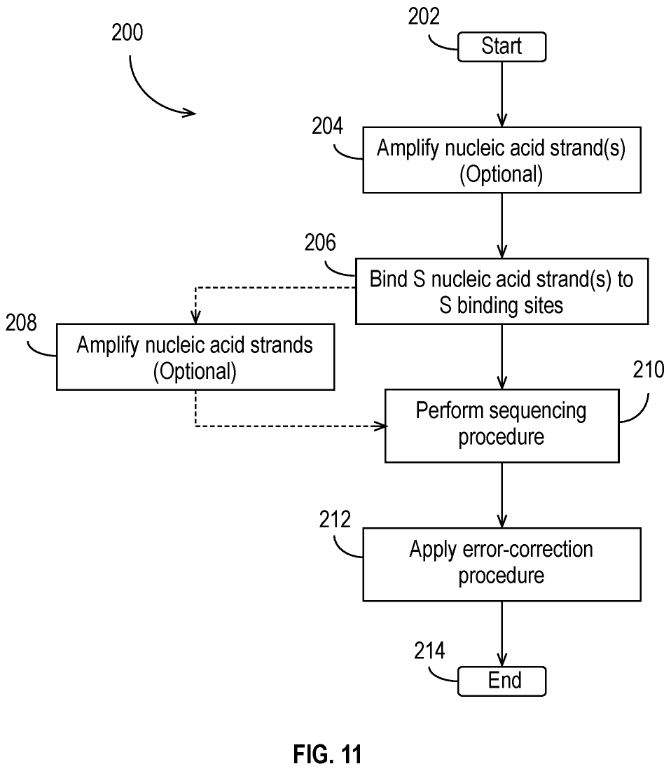

An exemplary method 200 of sequencing a plurality of nucleic acid strands (e.g., ssDNA) using a SMAS device 100 is illustrated in FIG. 11. At 202, the method begins. At 204, one or more nucleic acid strands may optionally be amplified prior to being added to the SMAS device 100. At 206, a plurality of S nucleic acid strands are bound to a plurality of S binding sites 116 of the SMAS device 100 (where the plurality includes at least two but not necessarily all of the binding sites 116 of the SMAS device 100). Optionally, at 208, the nucleic acid strands are amplified (e.g., via bridge amplification, which can be performed either in addition to or instead of the amplification at 204). At 210, a sequencing procedure is performed. The sequencing procedure may be, for example, the additive approach, the subtractive approach, or the modified additive approach described further below. The sequencing procedure performed at 210 produces S records, each of the S records capturing a number M of detection results for one of the plurality of S sensors (where, again, the plurality includes at least two but not necessarily all of the sensors 105 of the SMAS device 100, and the M detection results may comprise as few as one detection result, some subset of the total number of detection results obtained during the sequencing procedure, or all of the detection results obtained during the sequencing procedure). Each of the M detection results indicates whether, during a respective step of the M inquiry steps, the sensor 105 to which the record corresponds detected at least one label. The M detection results may be stored in a record, which may be stored in memory. At 212, an error-correction procedure is performed, as described further below. The error-correction procedure may comprise deterministic and/or probabilistic error-correction techniques. The error-correction procedure may be performed, for example, by the at least one processor 130 of the SMAS device 100. Alternatively, it may be performed by a processor that is external to the SMAS device 100 (e.g., an off-device processor, such as in an external computer). The error-correction procedure may be performed as the sequencing procedure is ongoing (e.g., in real-time or near-real-time), or it may be performed at some later time. At 214, the method 200 ends.

As noted above, at 210, a variety of protocols can be implemented to read nucleic acid sequences (e.g., DNA sequences) using a SMAS device 100. To simplify the analysis, it is assumed that the plurality of S sensors 105 of a SMAS device 100 detect only the presence or absence of a label 102 and do not distinguish between nucleotides based on detected signal levels. As a result, in some embodiments, the record of each sensor 105's detection results contains only “Yes” or “No” (or I/O or any other binary indicator) indications of whether, during a particular inquiry step, the sensor 105 detected a label or did not detect a label. It is to be appreciated that other approaches are possible and are within the scope of the disclosures herein. For example, different labels 102 could be attached to different nucleotides. As another example, rather than a binary “Yes” or “No” decision, a value of a characteristic could be detected (e.g., a resistance, frequency, intensity, etc.) and/or recorded, and a decision made on that basis as to whether a label was detected. For example, instead of having merely 0 and 1 (or “No” and “Yes”) as possible outputs of the sequencing procedure, the use of different labels for different nucleotides can result in one of five levels: 0 (no label detected), level 1 (label 1 detected), level 2 (label 2 detected), level 3 (label 3 detected), and level 4 (label 4 detected). In such cases, ranges of detected characteristics can be defined to distinguish whether a label was detected at all and, if so, which label was detected (e.g., if the value of the characteristic is between 0 and a first value, it is determined that no label was detected; if the value of the characteristic is between the first value and a second value, it is determined that the first label was detected; if the value of the characteristic is between the second value and a third value, it is determined that the second label was detected; etc.).

Below are explanations of three examples of DNA sequencing protocols, each comprising repeated inquiry cycles, each inquiry cycle having four inquiry steps. During each inquiry cycle, four binary “Yes” or “No” questions are answered for each ssDNA being sequenced. In one inquiry step, the question “Is the detected base adenine?” (“A?”) is answered. In another inquiry step, the question “Is the detected base thymine?” (“T?”) is answered. In another inquiry step, the question “Is the detected base cytosine?” (“C?”) is answered. And in another inquiry step, the question “Is the detected base guanine?” (“G?”) is answered. A record of the detection results obtained during the sequencing procedure can be created as inquiry cycles comprising the A?⇒T?⇒C?⇒G? inquiry steps are repeated. It is to be appreciated that the described order in which the nucleotides are introduced and the bases are detected is arbitrary (meaning that the order of the inquiry steps is arbitrary), and that the ordering in which the bases are tested in the examples herein (A?⇒T?⇒C?⇒G?) is merely exemplary.

Additive Approach

In the additive approach, the sensors 105 detect nanoscale labels 102 bound to nucleotides with cleavable linkers. All four types of nucleotides carry the same type of label 102 (e.g., molecular, fluorescent, magnetic, etc.) and use the same type of cleavable linker. An inquiry cycle that will result in four detection results, one of which will, absent errors, be a label detection for each of a plurality of S nucleic acid strands 101, involves the following steps according to one embodiment:
1. Obtain a baseline characteristic of each of a plurality of S sensors 105 (e.g., by measuring a baseline signal at each of a plurality of S sensors 105) of the SMAS device 100 (which may be all or fewer than all of the sensors 105 in the sensor array 110).
2. Introduce and incorporate labeled A nucleotides. Rinse off unbound labeled molecules.
3. Inquiry step 1: Obtain a characteristic of each of the plurality of S sensors 105 (e.g., by detecting a signal at each of the plurality of S sensors 105) and determine whether each sensor 105 detected at least one label. Save the detection result for each sensor 105 in position in a record corresponding to inquiry step 1 of the current inquiry cycle.
4. Introduce and incorporate labeled T nucleotides. Rinse off unbound labeled molecules.
5. Inquiry step 2: Obtain the characteristic of each of the plurality of S sensors 105 (e.g., by detecting the signal at each of the plurality of S sensors 105) and determine whether each sensor 105 detected at least one label. Save the detection result for each sensor 105 in position in a record corresponding to inquiry step 2 of the current inquiry cycle.
6. Introduce and incorporate labeled C nucleotides. Rinse off unbound labeled molecules.
7. Inquiry step 3: Obtain the characteristic of each of the plurality of S sensors 105 (e.g., by detecting the signal at each of the plurality of S sensors 105) and determine whether each sensor 105 detected at least one label. Save the detection result for each sensor 105 in position in a record corresponding to inquiry step 3 of the current inquiry cycle.
8. Introduce and incorporate labeled G nucleotides. Rinse off unbound labeled molecules.
9. Inquiry step 4: Obtain the characteristic of each of the plurality of S sensors 105 (e.g., by detecting the signal at each of the plurality of S sensors 105) and determine whether each sensor 105 detected at least one label. Save the detection result for each sensor 105 in position in a record corresponding to inquiry step 4 of the current inquiry cycle.
10. Cleave and rinse off labels from A, T, C, and G nucleotides.

Steps 1 through 10 can then be repeated for the next inquiry cycle. It is to be appreciated that the ordering of certain of the steps 1 through 10 is exemplary, and further that the number and numbering of steps 1 through 10 is for convenience and could be modified. As an example, and as previously explained, the order in which the nucleotides are introduced is arbitrary. As another example, steps 2, 4, 6, and 8 include introduction and incorporation of nucleotides, and rinsing off of unbound nucleotides as a single step, but it is to be appreciated that each of steps 2, 4, 6, and 8 can be broken into a series of smaller steps. Similarly, steps 3, 5, 7, and 9 can be further broken down into a series of smaller steps (e.g., obtain the characteristic, determine whether a label was detected, save the detection result). Conversely, steps could be combined (e.g., steps 2 and 3 could be combined, steps 4 and 5 could be combined, etc.).

It is to be appreciated that if it is likely that no errors occur during any inquiry cycle of the additive approach, it is possible to call (determine) the respective bases for the individual strands as soon as a label is detected. For example, referring to the steps above, if, at inquiry step 1 involving labeled A nucleotides, for a particular sensor 105, the obtained characteristic indicates that a sensor 105 detected a label, then saving the detection result may amount to calling the base complementary to A (T) for that sensor 105 (and binding site 116). Similarly, if, at inquiry step 2 involving labeled T nucleotides, for a particular sensor 105, the obtained characteristic indicates that the sensor 105 detected a label, then saving the detection result may amount to calling the base complementary to T (A) for that sensor 105 (and binding site 116). Likewise, if, at inquiry step 3 involving labeled C nucleotides, for a particular sensor 105, the obtained characteristic indicates that the sensor 105 detected a label, then saving the detection result may amount to calling the base complementary to C (G) for that sensor 105 (and binding site 116). Finally, if, at inquiry step 4 involving labeled G nucleotides, for a particular sensor 105, the obtained characteristic indicates that the sensor 105 detected a label, then saving the detection result may amount to calling the base complementary to G (C) for that sensor 105 (and binding site 116). As explained in further detail below, however, there are several types of errors that can occur during the sequencing procedure (e.g., during the additive approach), and therefore, in some embodiments, records are created during the sequencing procedure to record label detections/non-detections during each inquiry step of each inquiry cycle. An error-correction procedure can then be applied to some or all of the records before calling the bases.

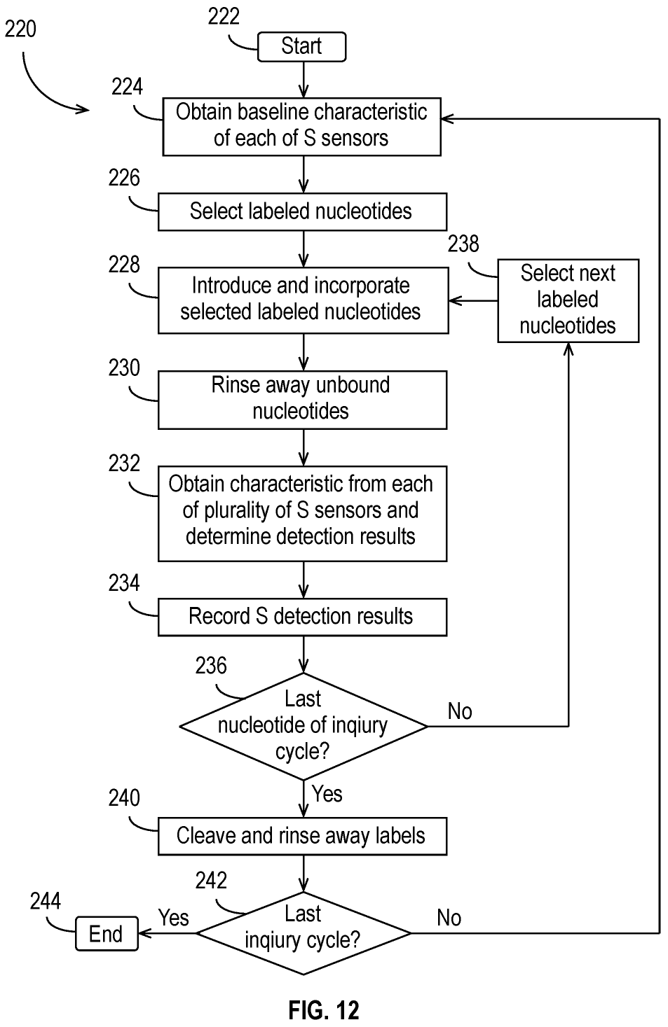

FIG. 12 is a flow diagram of a sequencing procedure 220 using the additive approach in accordance with some embodiments. The sequencing procedure 220 may be, for example, the sequencing procedure that is performed at step 210 of the exemplary method 200 of sequencing a plurality of nucleic acid strands (e.g., ssDNA) using a SMAS device 100 shown and described in the discussion of FIG. 11. At 222, the sequencing procedure 220 begins. At 224, a baseline characteristic of each of the S sensors 105 is obtained (e.g., by the at least one processor 130 of the SMAS device 100 with the assistance of the circuitry 120). When the inquiry cycle begins, at 226, a first labeled nucleotide is selected (e.g., referring to steps 1-10 above, the first labeled nucleotide would be A). At 228, the selected labeled nucleotide is introduced into the fluid chamber 115 and nucleotides are potentially incorporated into nucleic acid strands bound to binding sites 116. At 230, unbound nucleotides are rinsed away. At 232, the characteristic is obtained from each of the plurality of S sensors, and a detection result (e.g., label detected or label not detected) is determined for each of the plurality of S sensors 105. At 234, the S detection results are recorded in S records (e.g., as a 1 to indicate a label was detected or as a 0 to indicate no label was detected). At 236, it is determined whether the last-tested nucleotide was the last nucleotide of the inquiry cycle. For the example ordering of nucleotide testing assumed in steps 1-10 above, it would be determined at 236 (e.g., by the at least one processor 130) whether G was the last-tested nucleotide. If not, then at 238 the next labeled nucleotide to be tested in the inquiry cycle is selected, and steps 228 through 236 are repeated until it is determined at 236 that the last-tested nucleotide is the last nucleotide of the inquiry cycle. At 240, the labels are cleaved and rinsed away. At 242, it is determined (e.g., by the at least one processor 130), whether the last-completed inquiry cycle is the last inquiry cycle of the sequencing procedure 220. For example, the at least one processor 130 may determine whether enough detection results have been recorded to enable the at least one processor 130 (or some other processing entity, such as an external processor) to call a target number of bases (e.g., 150 bases). If not, the sequencing procedure 220 returns to step 224. If so, the sequencing procedure 220 ends at 244. Again, as explained above, the order in which the nucleotides are tested is arbitrary.

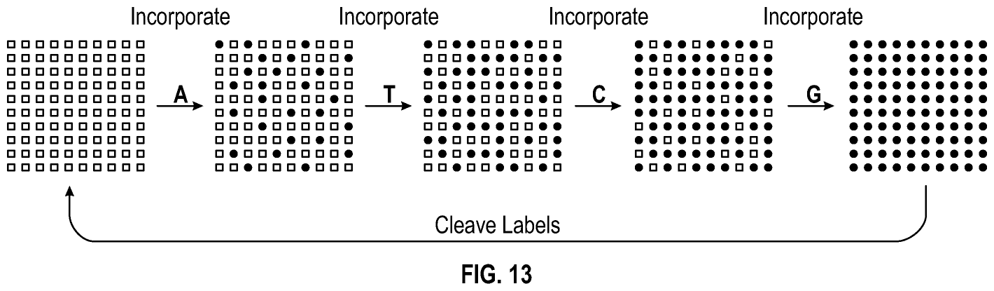

The additive sequencing protocol, which, in the exemplary case of DNA sequencing, comprises four nucleotide incorporations and one label cleaving reaction, is summarized in FIG. 13. The left-most panel of FIG. 13 illustrates a sensor array 110 having a total of 100 individual sensors 105, which are shown as squares. For purposes of the illustration, each of the 100 binding sites 116 in the sensor array 110 is assumed to hold a respective DNA strand, and each DNA strand is sensed by a respective sensor 105 (in other words, the binding sites 116 and sensors 105 are in a one-to-one relationship). Some of the DNA strands may be copies of others. Labeled nucleotides are added, one type at a time, to the fluid chamber 115, and labels are cleaved simultaneously after nucleotides are incorporated. Absent errors, a base-call can be accomplished after five reactions, namely, four nucleotide incorporations and one base-cleaving reaction. If errors occur, an error-correction procedure, as described below, may be applied.

Subtractive Approach

In the subtractive approach, the sensors 105 detect nanoscale labels 102 bound to nucleotides with cleavable linkers. All four types of nucleotides carry the same type of label (e.g., molecular, fluorescent, magnetic, etc.), but each has a different type of cleavable linker. An inquiry cycle that, absent errors, will result in four detection results, one of which will, absent errors, be a label detection for each of a plurality of S nucleic acid strands 101, involves the following steps in one embodiment:
1. Simultaneously introduce labeled A, T, C, and G nucleotides, incorporate, and rinse unbound labeled molecules. Obtain a baseline characteristic of each of the plurality of S sensors 105 (e.g., by detecting a signal at each of the plurality of S sensors 105). Absent errors, all sensors 105 will be detecting labels.
2. Inquiry step 1: Introduce a reagent (e.g., an enzyme) that cleaves labels only from a first nucleotide, e.g., A, rinse, and obtain the characteristic (e.g., measure the signal) at each of the plurality of S sensors 105. Determine (e.g., based on a change in the baseline characteristic) which sensors 105 are no longer detecting labels. Save the detection result for each sensor 105 in a position in a record corresponding to inquiry step 1 of the current inquiry cycle.
3. Inquiry step 2: Introduce a reagent that cleaves labels only from a second nucleotide, e.g., T, rinse, and obtain the characteristic (e.g., measure the signal) at each of the plurality of S sensors 105. Determine (e.g., based on a change in the baseline characteristic) which sensors 105 are no longer detecting labels. Save the detection result for each sensor 105 in a position in a record corresponding to inquiry step 2 of the current inquiry cycle.
4. Inquiry step 3: Introduce a reagent that cleaves labels only from a third nucleotide, e.g., C, rinse, and obtain the characteristic (e.g., measure the signal) at each of the plurality of S sensors 105. Determine (e.g., based on a change in the baseline characteristic) which sensors 105 are no longer detecting labels. Save the detection result for each sensor 105 in a position in a record corresponding to inquiry step 3 of the current inquiry cycle.
5. Inquiry step 4: Introduce a reagent that cleaves labels only from a fourth nucleotide, e.g., G, rinse, and obtain the characteristic (e.g., measure the signal) at each of the plurality of S sensors 105. Determine (e.g., based on a change in the baseline characteristic) which sensors 105 are no longer detecting labels. Save the detection result for each sensor 105 in a position in a record corresponding to inquiry step 4 of the current inquiry cycle.

Steps 1 through 5 can be repeated for the next inquiry cycle. It is to be appreciated that the ordering of certain of the steps 1 through 5 is exemplary, and further that the number and numbering of steps 1 through 5 is for convenience and could be modified. As an example, and as previously explained, the order in which the nucleotides are cleaved is arbitrary. Similarly, in step 1, the nucleotides could be introduced in turn (not necessarily simultaneously). As another example, inquiry steps 1, 2, 3, and 4 include introduction of a reagent, rinsing, obtaining the characteristic, determining which sensors are no longer (or are still) detecting labels, and saving the result as a single step, but it is to be appreciated that each inquiry step can be broken into a series of smaller steps.

It is to be appreciated that if it is likely that no errors occur during any inquiry cycle of the subtractive approach, it is possible to call (determine) the respective bases for the individual strands as soon as a label removal (the absence of a label) is first detected. For example, referring to the steps above, if, at inquiry step 1 involving labeled A nucleotides, for a particular sensor 105, the obtained characteristic indicates that a sensor 105 is no longer detecting a label, then saving the detection result may amount to calling the base complementary to A (T) for that sensor 105 (and binding site 116). Similarly, if, at inquiry step 2 involving labeled T nucleotides, for a particular sensor 105, the obtained characteristic indicates that a sensor 105 is no longer detecting a label, then saving the detection result may amount to calling the base complementary to T (A) for that sensor 105 (and binding site 116). Likewise, if, at inquiry step 3 involving labeled C nucleotides, for a particular sensor 105, the obtained characteristic indicates that a sensor 105 is no longer detecting a label, then saving the detection result may amount to calling the base complementary to C (G) for that sensor 105 (and binding site 116). Finally, if, at inquiry step 4 involving labeled G nucleotides, for a particular sensor 105, the obtained characteristic indicates that a sensor 105 is no longer detecting a label, then saving the detection result may amount to calling the base complementary to G (C) for that sensor 105 (and binding site 116). As explained in further detail below, however, there are several types of errors that can occur during the sequencing procedure (e.g., during the subtractive approach), and therefore, in some embodiments, records are created during the sequencing procedure to record label detections/non-detections during each inquiry step of each inquiry cycle. An error-correction procedure can then be applied to some or all of the records before calling the bases.

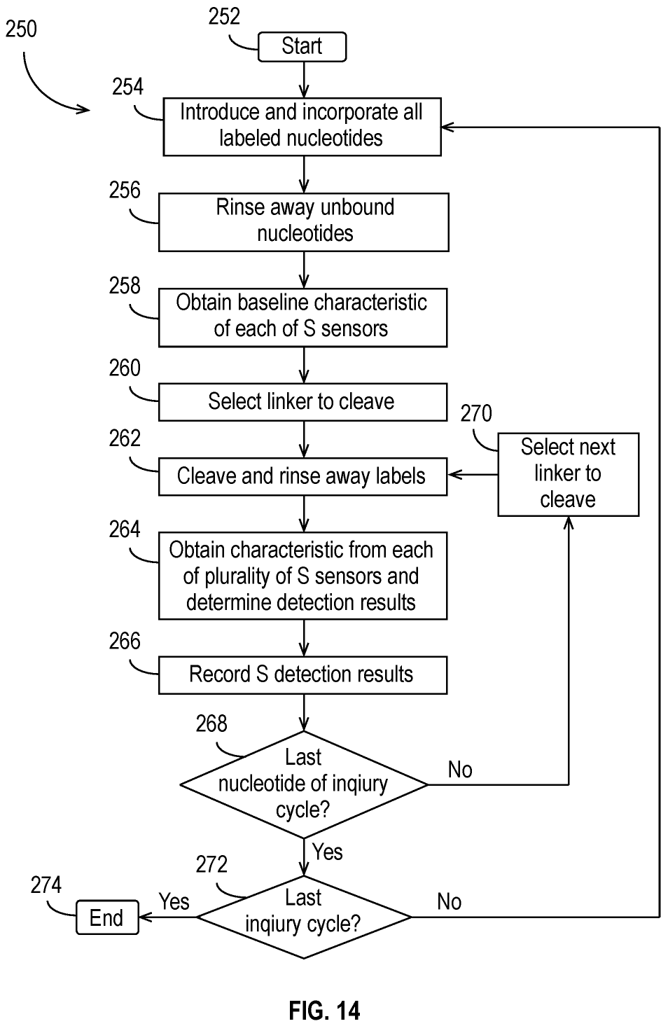

FIG. 14 is a flow diagram of a sequencing procedure 250 using the subtractive approach in accordance with some embodiments. The sequencing procedure 250 may be, for example, the sequencing procedure that is performed at step 210 of the exemplary method 200 of sequencing a plurality of nucleic acid strands (e.g., ssDNA) using a SMAS device 100 shown and described in the discussion of FIG. 11. The sequencing procedure 250 begins at 252. At 254, all of the labeled nucleotides are introduced into the fluid chamber 115 and nucleotides are incorporated into nucleic acid strands bound to the S binding sites 116. At 256, unbound nucleotides are rinsed away. At 258, a baseline characteristic of each of the S sensors 105 is obtained (e.g., by the at least one processor 130 of the SMAS device 100 with the assistance of the circuitry 120). Assuming a nucleotide has been incorporated into the nucleic acid strand bound to each of the S binding sites, the obtained characteristics represents the characteristics of the sensors 105 when they are detecting at least one label. At 260, one of the cleavable linkers is selected for cleavage (or, equivalently, one of the nucleotides is selected). At 262, the labels attached to the selected nucleotide are cleaved and rinsed away. Assuming no errors, following step 262, the sensors 105 sensing those nucleic acid strands that incorporated the tested nucleotide (e.g., the one to which labels were attached by the selected cleavable linker) will exhibit a change in the characteristic (e.g., a change in a signal associated with or generated by the sensor 105). At 264, the characteristic is obtained from each of the plurality of S sensors, and a detection result (e.g., label detected or label not detected) is determined for each of the plurality of S sensors 105. At 266, the S detection results are recorded in S records (e.g., as a 1 to indicate a label was detected or as a 0 to indicate no label was detected). At 268, it is determined whether the last-tested nucleotide was the last nucleotide of the inquiry cycle. For the example ordering of nucleotide testing assumed in steps 1-5 above, it would be determined at 268 (e.g., by the at least one processor 130) whether G was the last-tested nucleotide. If not, then at 270 the next cleavable linker to be cleaved (or, equivalently, the next nucleotide to be tested) in the inquiry cycle is selected, and steps 262 through 268 are repeated until it is determined at 268 that the last-cleaved linker (or, equivalently, the last-tested nucleotide) is the last linker (or nucleotide) of the inquiry cycle. At 272, it is determined (e.g., by the at least one processor 130), whether the last-completed inquiry cycle is the last inquiry cycle of the sequencing procedure 250. For example, the at least one processor 130 may determine whether enough detection results have been recorded to enable the at least one processor 130 (or some other processing entity, such as an external processor) to call a target number of bases (e.g., 150 bases). If not, the sequencing procedure 250 returns to step 254. If so, the sequencing procedure 250 ends at 274. Again, as explained above, the order in which the nucleotides are tested is arbitrary.

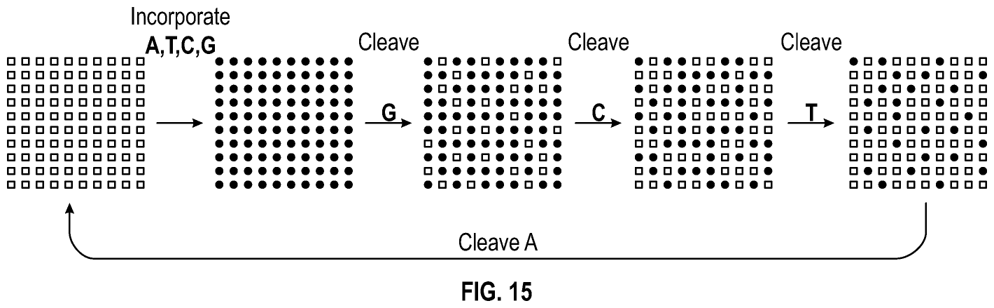

The subtractive sequencing protocol, which, in the exemplary case of DNA sequencing, comprises one nucleotide incorporation and four base cleaving reactions, is summarized in FIG. 15. The left-most panel of FIG. 15 illustrates a sensor array 110 having a total of 100 individual sensors 105, which are shown as squares. For purposes of the illustration, each of the 100 binding sites 116 in the sensor array 110 is assumed to hold a respective DNA strand, and each DNA strand is sensed by a respective sensor 105 (in other words, the binding sites 116 and sensors 105 are in a one-to-one relationship). Some of the DNA strands may be copies of others. All four types of labeled nucleotides are added simultaneously to the fluid chamber 115, and labels are removed after incorporation, one type of nucleotide (e.g., cleavable linker) at a time. Absent errors, a base-call can be accomplished after five reactions, namely, one nucleotide incorporation and four base cleaving reactions. If errors occur, an error-correction procedure, as described below, may be applied.

Modified Additive Approach

In the modified additive approach, the sensors 105 detect nanoscale labels 102 bound to nucleotides with cleavable linkers. All four types of nucleotides carry the same type of label 102 (e.g., molecular, fluorescent, magnetic, etc.) and use the same type of cleavable linker. Labeled nucleotides are added separately, and, after the addition of each nucleotide, the presence of labels 102 is detected. An inquiry cycle that, absent errors, will result in four detection results, at least one of which will be a label detection, for each of a plurality of S nucleic acid strands 101 involves the following steps in one embodiment:
1. Obtain a baseline characteristic for each of a plurality of S sensors 105 (e.g., by measuring a baseline signal at each of the plurality of S sensors 105) of the SMAS device 100 (which may be all or fewer than all of the sensors 105 in the sensor array 110).
2. Introduce and incorporate a first labeled nucleotide, e.g., labeled A nucleotides. Rinse off unbound labeled molecules.
3. Inquiry step 1: Obtain a characteristic of each of the plurality of S sensors 105 (e.g., by detecting a signal at each of the plurality of S sensors 105) and determine whether each sensor 105 detected at least one label. Save the detection result for each sensor 105 in a position in a record corresponding to inquiry step 1 of the current inquiry cycle.
4. Cleave and rinse off labels.
5. Introduce and incorporate a second labeled nucleotide, e.g., labeled T nucleotides. Rinse off unbound labeled molecules.
6. Inquiry step 2: Obtain a characteristic of each of the plurality of S sensors 105 (e.g., by detecting a signal at each of the plurality of S sensors 105) and determine whether each sensor 105 detected at least one label. Save the detection result for each sensor 105 in a position in a record corresponding to inquiry step 2 of the current inquiry cycle.
7. Cleave and rinse off labels.
8. Introduce and incorporate a third labeled nucleotide, e.g., labeled C nucleotides. Rinse off unbound labeled molecules.
9. Inquiry step 3: Obtain a characteristic of each of the plurality of S sensors 105 (e.g., by detecting a signal at each of the plurality of S sensors 105) and determine whether each sensor 105 detected at least one label. Save the detection result for each sensor 105 in a position in a record corresponding to inquiry step 3 of the current inquiry cycle.
10. Cleave and rinse off labels.
11. Introduce and incorporate a fourth labeled nucleotide, e.g., labeled G nucleotides. Rinse off unbound labeled molecules.
12. Inquiry step 4: Obtain a characteristic of each of the plurality of S sensors 105 (e.g., by detecting a signal at each of the plurality of S sensors 105) and determine whether each sensor 105 detected at least one label. Save the detection result for each sensor 105 in a position in a record corresponding to inquiry step 4 of the current inquiry cycle.
13. Cleave and rinse off labels.

Steps 1 through 13 may then be repeated for the next inquiry cycle. It is to be appreciated that the ordering of certain of the steps 1 through 13 is exemplary, and further that the number and numbering of steps 1 through 13 is for convenience and could be modified. As an example, and as previously explained, the order in which the nucleotides are introduced is arbitrary. As another example, steps 2, 5, 8, and 11 include introduction and incorporation of nucleotides, and rinsing off of unbound nucleotides as a single step, but it is to be appreciated that each of steps 2, 5, 8, and 11 can be broken into a series of smaller steps. Similarly, steps 3, 6, 9, and 12 (inquiry steps 1, 2, 3, and 4, respectively) can be further broken down into a series of smaller steps (e.g., obtain the characteristic, determine whether a label was detected, save the detection result). Conversely, steps could be combined (e.g., steps 2 and 3 could be combined, steps 3 and 4 could be combined, steps 2-4 could be combined, steps 5 and 6 could be combined, steps 6 and 7 could be combined, steps 5-7 could be combined, etc.).

It is to be appreciated that if it is likely that no errors occur during any inquiry cycle of the modified additive approach, it is possible to call (determine) the respective bases for the individual strands as soon as a label is detected. For example, referring to the steps above, if, at inquiry step 1 involving labeled A nucleotides, for a particular sensor 105, the obtained characteristic indicates that a sensor 105 detected a label, then saving the detection result may amount to calling the base complementary to A (T) for that sensor 105 (and binding site 116). Similarly, if, at inquiry step 2 involving labeled T nucleotides, for a particular sensor 105, the obtained characteristic indicates that a sensor 105 detected a label, then saving the detection result may amount to calling the base complementary to T (A) for that sensor 105 (and binding sites 116). Likewise, if, at inquiry step 3 involving labeled C nucleotides, for a particular sensor 105, the obtained characteristic indicates that a sensor 105 detected a label, then saving the detection result may amount to calling the base complementary to C (G) for that sensor 105 (and binding site 116). Finally, if, at inquiry step 4 involving labeled G nucleotides, for a particular sensor 105, the obtained characteristic indicates that a sensor 105 detected a label, then saving the detection result may amount to calling the base complementary to G (C) for that sensor 105 (and binding site 116). As explained in further detail below, however, there are several types of errors that can occur during the sequencing procedure (e.g., during the additive approach), and therefore, in some embodiments, records are created during the sequencing procedure to record label detections/non-detections during each inquiry step of each inquiry cycle. An error-correction procedure can then be applied to some or all of the records before calling the bases.

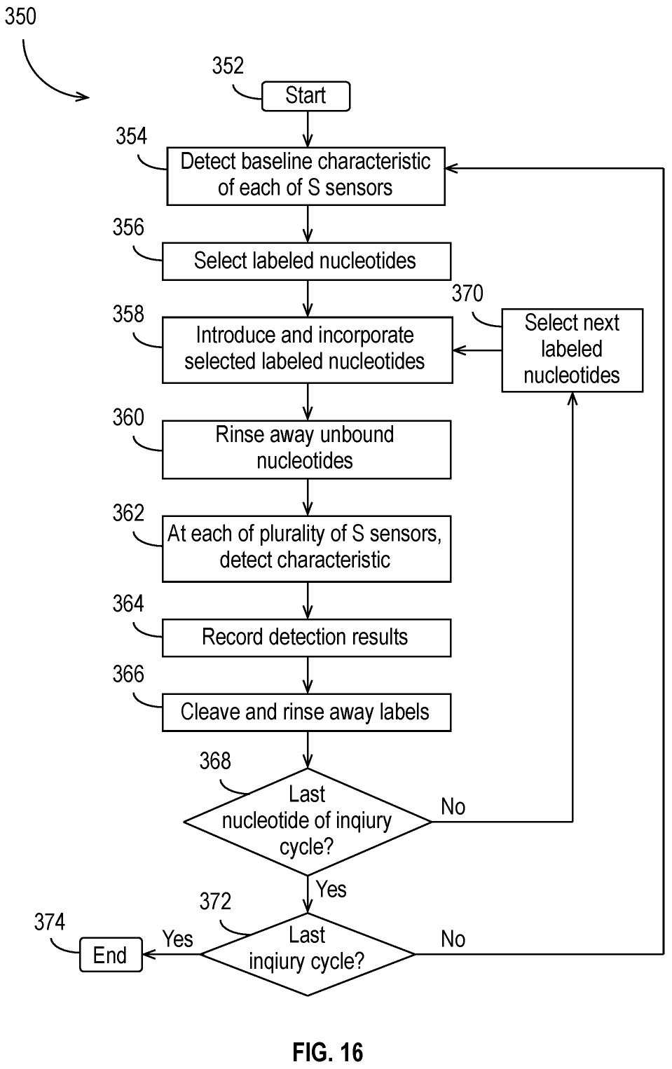

FIG. 16 is a flow diagram of a sequencing procedure 350 using the modified additive approach in accordance with some embodiments. The sequencing procedure 350 may be, for example, the sequencing procedure that is performed at step 210 of the exemplary method 200 of sequencing a plurality of nucleic acid strands (e.g., ssDNA) using a SMAS device 100 shown and described in the discussion of FIG. 11. At 352, the sequencing procedure 350 begins. At 354, a baseline characteristic of each of the S sensors 105 is obtained (e.g., by the at least one processor 130 of the SMAS device 100 with the assistance of the circuitry 120). When the inquiry cycle begins, at 356, a first labeled nucleotide is selected (e.g., referring to steps 1-13 above, the first labeled nucleotide would be A). At 358, the selected labeled nucleotide is introduced into the fluid chamber 115 and nucleotides are potentially incorporated into nucleic acid strands bound to binding sites 116. At 360, unbound nucleotides are rinsed away. At 362, the characteristic is obtained from each of the plurality of S sensors, and a detection result (e.g., label detected or label not detected) is determined for each of the plurality of S sensors 105. At 364, the S detection results are recorded in S records (e.g., as a 1 to indicate a label was detected or as a 0 to indicate no label was detected). At 366, the labels are cleaved and rinsed away. At 368, it is determined whether the last-tested nucleotide was the last nucleotide of the inquiry cycle. For the example ordering of nucleotide testing assumed in steps 1-13 above, it would be determined at 368 (e.g., by the at least one processor 130) whether G was the last-tested nucleotide. If not, then at 370 the next labeled nucleotide to be tested in the inquiry cycle is selected, and steps 358 through 368 are repeated until it is determined at 368 that the last-tested nucleotide is the last nucleotide of the inquiry cycle. At 372, it is determined (e.g., by the at least one processor 130), whether the last-completed inquiry cycle is the last inquiry cycle of the sequencing procedure 350. For example, the at least one processor 130 may determine whether enough detection results have been recorded to enable the at least one processor 130 (or some other processing entity, such as an external processor) to call a target number of bases (e.g., 150 bases). If not, the sequencing procedure 350 returns to step 354. If so, the sequencing procedure 350 ends at 374. Again, as explained above, the order in which the nucleotides are tested is arbitrary.

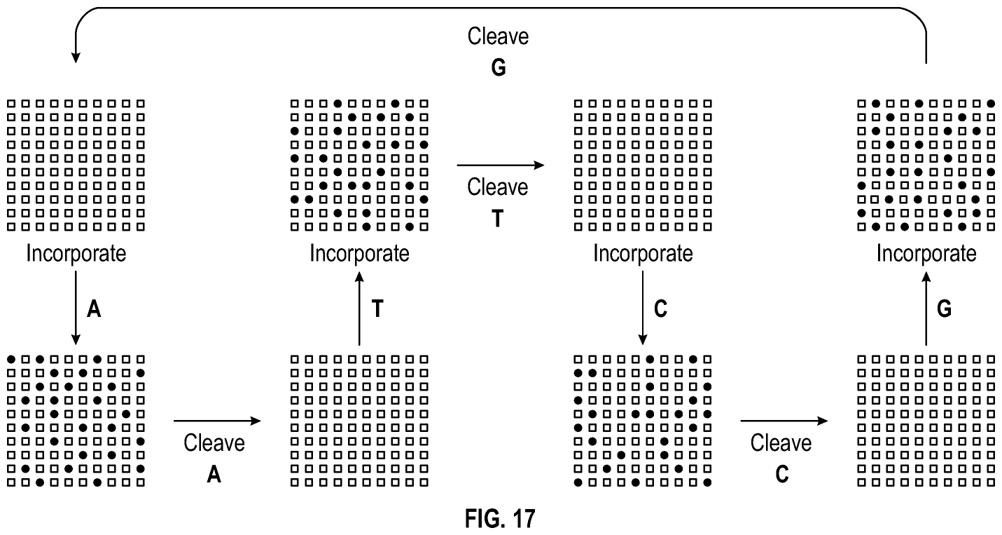

The modified additive sequencing protocol, which, in the exemplary case of DNA sequencing, comprises four nucleotide incorporations and four base cleaving reactions, is illustrated in FIG. 17. The left-most panel of FIG. 17 illustrates a sensor array 110 having a total of 100 individual sensors 105, which are shown as squares. For purposes of the illustration, each of the 100 binding sites 116 in the sensor array 110 is assumed to hold a respective DNA strand, and each DNA strand is sensed by a respective sensor 105 (in other words, the binding sites 116 and sensors 105 are in a one-to-one relationship). Some of the DNA strands may be copies of others. As shown and described, labeled nucleotides are added to the fluid chamber 115 one type at a time, and labels are cleaved after incorporation and label detection. Absent errors, a base-call can be accomplished, on average, after 5 reactions, namely, 2.5 nucleotide incorporations and 2.5 base cleaving reaction.

Thus, absent errors, for DNA sequencing the modified additive approach yields at least one base-call per ssDNA after 8 reactions (4 nucleotide incorporations and 4 base cleavages) to test for all the bases. On average, however, a base-call is made after only 5 reactions (2.5 nucleotide incorporations and 2.5 base cleavages). Because labels are removed after introduction of every nucleotide, multiple nucleotides can be incorporated and called during a single A?⇒T?⇒C?⇒G? inquiry cycle. Specifically, in an unknown ssDNA sequence there is a 1 in 4 chance the unknown base is T. If the base happens to be T, it will be detected at the third step following one incorporation and one base cleaving reaction when the A nucleotide is introduced. There is a 1 in 4 chance the unknown base is A. If the base happens to be A, it will be detected at the fifth step of the inquiry cycle A?⇒T?, when the T nucleotide has been introduced and two incorporation and two cleavages have been performed. There is a 1 in 4 chance the unknown base is G. If the base happens to be G, it will be detected at the seventh step of the inquiry cycle A?⇒T?⇒C?, when the C nucleotide has been introduced and three incorporation and three cleavages have been performed. Finally, there is a 1 in 4 chance the unknown base is C. If the base happens to be C, it will be detected at the eleventh step of the inquiry cycle A?⇒T?⇒C?⇒G?, when the C nucleotide has been introduced and four incorporation and four cleavages have been performed. It therefore takes on average 2.5 inquiries (5 reactions)(¼×1+¼×2+¼×3+¼×4=2.5) to call a single unknown base. Alternatively, if the unknown 4-base sequence of a particular ssDNA happens to be the best-case scenario ATCG (for the selected order of introduced nucleotides assumed for this example), only one inquiry cycle A?⇒T?⇒C?⇒G? needs to be performed: 8 reactions (4 nucleotide incorporations and 4 base cleavages) in total, or 2 reactions per base-call. If, however, the unknown sequence happens to be, for example, GCTA, GGCT, GCTT, GGGG, etc., four inquiry cycles, each including all of A?⇒T?⇒C?⇒G?, need to be performed, resulting in a total of 32 reactions (16 nucleotide incorporations and 16 base cleavages), or 8 reactions per base-call. On average, however, for a random DNA sequence it takes 2.5 inquiries or 5 reactions (2.5 nucleotide incorporations and 2.5 base cleavages) to make a single base-call.

### Sources of Sequencing Errors

Ideally, sequencing procedures, whether in CLUS devices or SMAS devices 100, would be error-free. In other words, for example, nucleotides would always be properly labeled, nucleotides would always be correctly incorporated into DNA, all labels would be successfully cleaved during the cleavage steps, all cleaved labels would be successfully rinsed away, etc. In reality, however, errors can occur during any sequencing procedure. This section explores the sources of sequencing errors in both CLUS devices and SMAS devices 100 and describes error mitigation strategies for SMAS devices 100. As explained further below, error correction methods can be used to improve sequencing accuracy of SMAS devices 100.

Because the modified additive approach described above is a conceptually simple (and symmetric, in that each nucleotide is handled in the same way) sequencing procedure, it is a good model for explaining how errors propagate in both CLUS devices and in SMAS devices 100. Four sources of errors are considered, assuming nanoscale labels are attached to nucleotides via a cleavable linker. Each error occurs at a rate denoted as r, which has a value between 0 and 1. The four sources of error are:

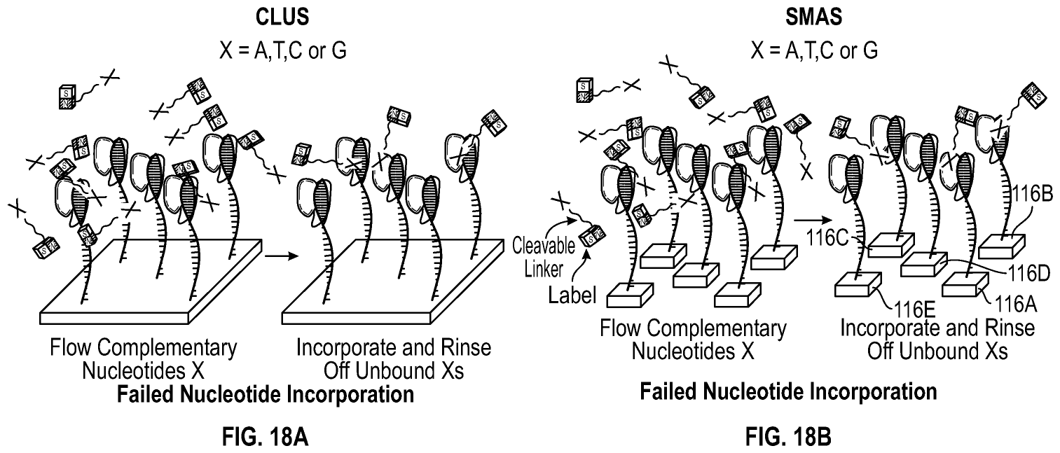

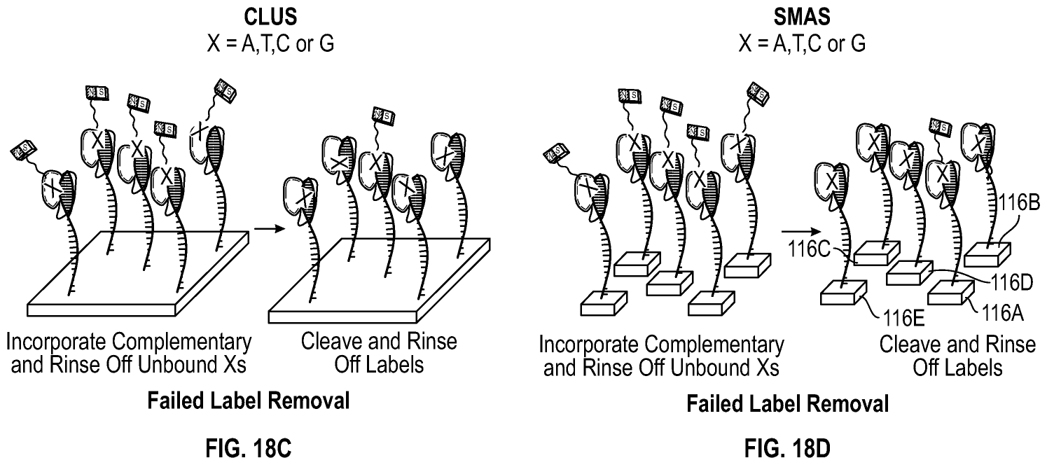

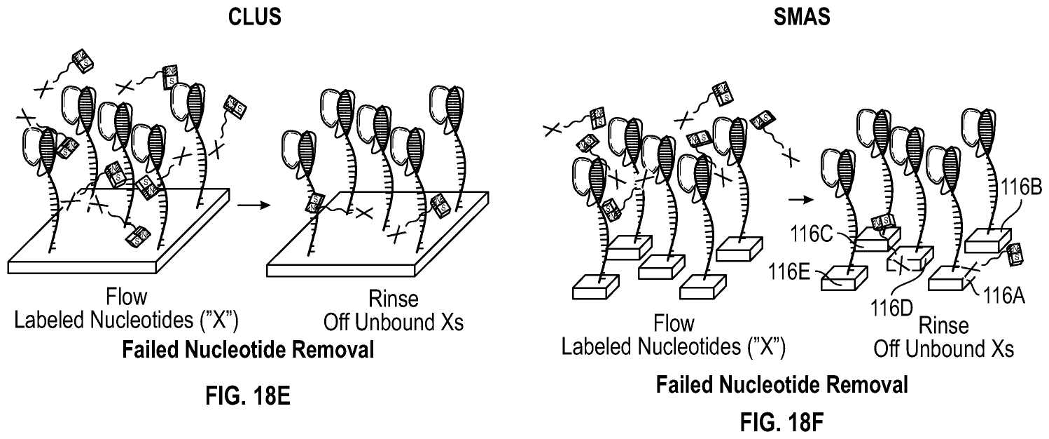

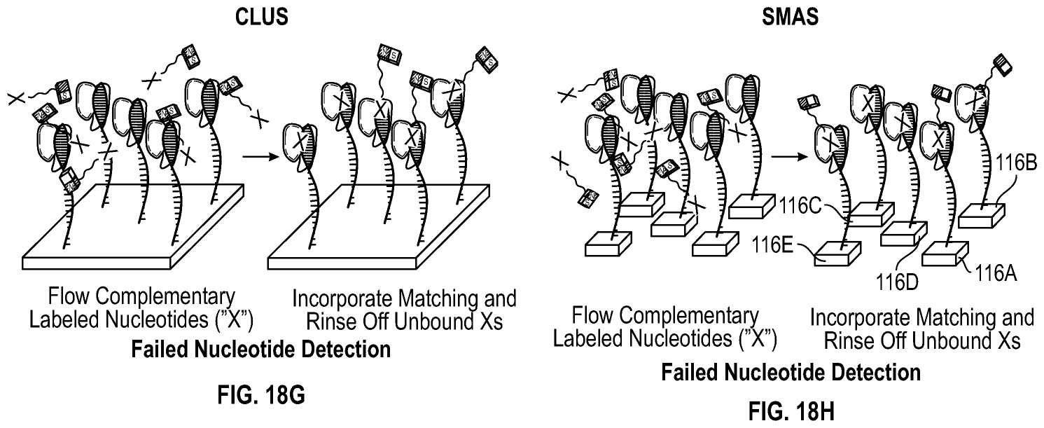

Failed Nucleotide Incorporation (FNI): Failed nucleotide incorporation (FNI) occurs when a properly labeled nucleotide molecule has not reached the ssDNA binding site, or polymerase failed to incorporate it. FIG. 18A illustrates FNI for a CLUS device that is sequencing five instances of a ssDNA. Following the flow of complementary nucleotides, only three of the five ssDNA have incorporated the labeled nucleotides (illustrated as having magnetic labels). Thus, two out of five nucleotides (r=0.4) fail to incorporate. FIG. 18B illustrates FNI for a SMAS device 100. Each of five binding sites 116 holds an instance of a ssDNA. Following the flow of complementary nucleotides, only three of the five ssDNA, those bound to binding sites 116A, 116B, and 116C, have incorporated the labeled nucleotides (illustrated, solely for purposes of example, as having magnetic labels). Again, two out of five ssDNA instances (r=0.4) fail to incorporate nucleotides.

Failed Label Removal (FLR): Failed label removal (FLR) results when a labeled nucleotide molecule is incorporated, but the label is not removed after label detection because the cleaving reagent has not reached the linker or has failed to cleave it. FIG. 18C illustrates FLR for the CLUS device described above in the discussion of FIG. 18A. After incorporation of complementary nucleotides and rinsing to remove unbound nucleotides, detecting labels, and cleaving and rinsing labels, one label remains attached to one of the ssDNA instances (r=0.2). Similarly, in FIG. 18D, which illustrates FLR for the SMAS device 100 described above in the discussion of FIG. 18B, after incorporation of complementary nucleotides and rinsing to remove unbound nucleotides, detecting labels, and cleaving and rinsing labels (e.g., steps 2-4, 5-7, 8-10, and/or 11-13 described above), a label remains attached to the ssDNA at the binding site 116A (r=0.2).

Failed Nucleotide Removal (FNR): Failed nucleotide removal (FNR) results when a labeled nucleotide, whether complementary or non-complementary, binds non-specifically to the surface of the binding site 116 and/or sensor 105. FIG. 18E illustrates an example of FNR for the CLUS device described above in the discussion of FIG. 18A. After the flow of nucleotides and rinsing to remove unbound nucleotides, two rogue nucleotides and their labels remain on the surface of the binding site. Similarly, in FIG. 18F, which illustrates FNR for the SMAS device 100 described above in the discussion of FIG. 18B, after the flow of nucleotides and rinsing to remove unbound nucleotides, one rogue nucleotide remains on the surface of the binding site 116A, and another rogue nucleotide remains on the surface of the binding site 116D. In this example, for both the CLUS device and the SMAS device 100, r=0.4.

Failed Label Detection (FLD): Failed label detection (FLD) results when the correct complementary nucleotide is incorporated, but the label is not detected either because the label is missing or the sensor failed to recognize it. FIG. 18G illustrates FLD for the CLUS device described above in the discussion of FIG. 18A. After incorporation of complementary nucleotides and rinsing to remove unbound nucleotides, two of the ssDNA instances have incorporated complementary nucleotides, but the labels are missing (r=0.4). Similarly, in FIG. 18H, which illustrates FLD for the SMAS device 100 described above in the discussion of FIG. 18B, after incorporation of complementary nucleotides and rinsing to remove unbound nucleotides (e.g., step 2, 5, 8, or 11 described above), the labels that should be attached to the nucleotides incorporated in the ssDNA at the binding sites 116C and 116D are missing (r=0.4).

FIG. 18A through 18H illustrate the labels as magnets, thereby suggesting magnetic labels and magnetic sensors, but it is to be appreciated that, as explained above, the labels may be any type of detectable label (e.g., fluorescent, magnetic, etc.) and the sensors may be any type of sensors capable of detecting the selected type of label (e.g., optical, magnetic, organometallic, charged molecule, etc.).

It is assumed that the four error types (FNI, FLR, FNR, and FLD) occur at the same rate r, where 0<r<1; e.g., if r=0.01, then there is 1 failure in 100 on average. It is also assumed that the sensors 105 of a SMAS device 100 (e.g., nanoscale sensors 105) can detect a single label almost every time, and that the response of large cluster sensors used in CLUS devices is linear, e.g., the sensors of a CLUS device can distinguish between N and N+1 labeled strands for all values of N.

Cluster Sequencer Vs. Single-Molecule Array Sequencer: Qualitative Comparison and Error Correction

Disclosed herein are two types of error correction, referred to as deterministic error correction and probabilistic error correction. A SMAS device 100 may use one or both types of error correction, as explained further below.

As explained above, the modified additive approach is a good model for explaining how errors propagate and how the disclosed error correction algorithms can be implemented. It is to be understood that the disclosed error mitigation algorithms can also be applied when other sequencing approaches, such as the additive approach or the subtractive approach, are used.

Consider CLUS devices and SMAS devices 100 using the modified additive approach sequencing procedure with large error rates of r=0.1 (e.g., 1 out of 10 reactions fails) and a small number of instances of (ideally identical) strands, e.g., N=K=3, where the variable N denotes the cluster size used in the CLUS device, and the variable K denotes the number of sensors 105 of a SMAS device 100 that sense instances of the same DNA strand. (As explained previously, the K sensors may be near each other, or they may be scattered within the sensor array 110). To describe embodiments of deterministic error correction, initially only FNI and FLR errors are considered. FNI, FLR, and FLD errors are then considered, and error-mitigation strategies are described. Finally, all four types of errors are considered, and error-correction procedures that address all four types of errors are described.

When using a SMAS device 100, FLR errors can be detected and removed, whether in real time during the sequencing procedure or at some time afterward. FLR errors can be detected by obtaining the characteristic for each of the S sensors 105 after cleaving and rinsing the labels. FNI errors can be detected by inspecting each sensor 105's record and identifying inquiry cycles during which that sensor 105 failed to detect any label(s). Accordingly, the modified additive approach can be adjusted to add these detection steps as follows according to one embodiment:
1. Obtain a baseline characteristic for each of a plurality of S sensors 105 (e.g., by measuring a baseline signal at each of the plurality of S sensors 105) of the SMAS device 100 (which may be all or fewer than all of the sensors 105 in the sensor array 110).
2. Introduce and incorporate a first labeled nucleotide, e.g., labeled A nucleotides. Rinse off unbound labeled molecules.
3. Inquiry step 1: Obtain a characteristic of each of the plurality of S sensors 105 (e.g., by detecting a signal at each of the plurality of S sensors 105) and determine whether each sensor 105 detected at least one label. Save the detection result for each sensor 105 in a position in a record corresponding to inquiry step 1 of the current inquiry cycle.
4. Cleave and rinse off the labels.
5. Obtain the characteristic for each of the plurality of S sensors 105 that detected a label in step 3. If the obtained characteristic for any of those sensors 105 indicates that the sensor 105 is still detecting a label, chemistry has failed to cleave the label (e.g., for that sensor, there is a FLR error).
6. Introduce and incorporate a second labeled nucleotide, e.g., labeled T nucleotides. Rinse off unbound labeled molecules.
7. Inquiry step 2: Obtain a characteristic of each of the plurality of S sensors 105 (e.g., by detecting a signal at each of the plurality of S sensors 105) and determine whether each sensor 105 detected at least one label. Save the detection result for each sensor 105 in a position in a record corresponding to inquiry step 2 of the current inquiry cycle.
8. Cleave and rinse off labels.
9. Obtain the characteristic for each of the plurality of S sensors 105 that detected a label in step 7. If the obtained characteristic for any of those sensors 105 indicates that the sensor 105 is still detecting a label, chemistry has failed to cleave the label (e.g., for that sensor, there is a FLR error).
10. Introduce and incorporate a third labeled nucleotide, e.g., labeled C nucleotides. Rinse off unbound labeled molecules.
11. Inquiry step 3: Obtain a characteristic of each of the plurality of S sensors 105 (e.g., by detecting a signal at each of the plurality of S sensors 105) and determine whether each sensor 105 detected at least one label. Save the detection result for each sensor 105 in a position in a record corresponding to inquiry step 3 of the current inquiry cycle.
12. Cleave and rinse off labels.
13. Obtain the characteristic for each of the plurality of S sensors 105 that detected a label in step 11. If the obtained characteristic for any of those sensors 105 indicates that the sensor 105 is still detecting a label, chemistry has failed to cleave the label (e.g., for that sensor, there is a FLR error).
14. Introduce and incorporate a fourth labeled nucleotide, e.g., labeled G nucleotides. Rinse off unbound labeled molecules.
15. Inquiry step 4: Obtain a characteristic of each of the plurality of S sensors 105 (e.g., by detecting a signal at each of the plurality of S sensors 105) and determine whether each sensor 105 detected at least one label. Save the detection result for each sensor 105 in a position in a record corresponding to inquiry step 4 of the current inquiry cycle. If there are sensors 105 without an assigned base for the inquiry cycle (e.g., sensors 105 that failed to detect A, T, C, or G during the inquiry cycle), chemistry has failed to incorporate a nucleotide (e.g., for these sensors 105, there is FNI).
16. Cleave and rinse off labels.
17. Obtain the characteristic for each of the plurality of S sensors 105 that detected a label in step 15. If the obtained characteristic for any of those sensors 105 indicates that the sensor 105 is still detecting a label, chemistry has failed to cleave the label (e.g., for that sensor, there is a FLR error).

Steps 1 through 17 can then be repeated to for the next inquiry cycle (e.g., to estimate the next base or to re-read the current base if the prior inquiry cycle failed to read it). It is to be appreciated that the ordering of certain of the steps 1 through 17 is exemplary, and further that the number and numbering of steps 1 through 17 is for convenience and could be modified. As an example, and as previously explained, the order in which the nucleotides are introduced is arbitrary. As another example, steps 2, 6, 10, and 14 include introduction and incorporation of nucleotides, and rinsing off of unbound nucleotides as a single step, but it is to be appreciated that each of steps 2, 6, 10, and 14 can be broken into a series of smaller steps. Similarly, steps 3, 7, 11, and 15 (inquiry steps 1, 2, 3, and 4, respectively) can be further broken down into a series of smaller steps (e.g., obtain the characteristic, determine whether a label was detected, save the detection result). Likewise, although step 15 includes identifying FNI errors, that task could be made a separate step. Conversely, steps could be combined (e.g., some or all of steps 2-5, some or all of steps 6-9, some or all of steps 10-13, some or all of steps 14-17, etc.).

FIG. 19 is a flow diagram of an exemplary sequencing procedure 400 using the modified additive approach with FLR and FNI error detection in accordance with some embodiments. The sequencing procedure 400 may be, for example, the sequencing procedure that is performed at step 210 of the exemplary method 200 of sequencing a plurality of nucleic acid strands (e.g., ssDNA) using a SMAS device 100 shown and described in the discussion of FIG. 11. At 402, the sequencing procedure 400 begins. At 404, a baseline characteristic of each of the S sensors 105 is obtained (e.g., by the at least one processor 130 of the SMAS device 100 with the assistance of the circuitry 120). When the inquiry cycle begins, at 406, a first labeled nucleotide is selected (e.g., referring to steps 1-17 above, the first labeled nucleotide would be A). At 408, the selected labeled nucleotide is introduced into the fluid chamber 115 and nucleotides are potentially incorporated into nucleic acid strands bound to binding sites 116. At 410, unbound nucleotides are rinsed away. At 412, the characteristic is obtained from each of the plurality of S sensors, and a detection result (e.g., label detected or label not detected) is determined for each of the plurality of S sensors 105. At 414, the S detection results are recorded in S records (e.g., as a 1 to indicate a label was detected or as a 0 to indicate no label was detected). At 416, the labels are cleaved and rinsed away. At 418, the characteristic is obtained for those sensors 105 that detected labels during step 412/414. At 420, it is determined whether any of the sensors 105 that detected labels during step 412/414 are still detecting labels. If so, then at 422 it is determined that a FLR error has been detected for the sensors 105 that are still detecting at least one label, even though the labels were cleaved and rinsed away at 416. The sequencing procedure 400 then continues to 424. If, at 420, it is determined (e.g., by the at least one processor 130) that none of the sensors 105 that detected labels during step 412/414 are still detecting labels, the sequencing procedure also continues to 424. At 424, it is determined whether the last-tested nucleotide was the last nucleotide of the inquiry cycle. For the example ordering of nucleotide testing assumed in steps 1-17 above, it would be determined at 368 (e.g., by the at least one processor 130) whether G was the last-tested nucleotide. If not, then at 426 the next labeled nucleotide to be tested in the inquiry cycle is selected, and steps 408 through 420 (and, if applicable, 422) are repeated until it is determined at 424 that the last-tested nucleotide is the last nucleotide of the inquiry cycle. At 428, FNI errors are detected for those of the S sensors 105 that failed to detect any label during the last-completed inquiry cycle. At 430, it is determined (e.g., by the at least one processor 130), whether the last-completed inquiry cycle is the last inquiry cycle of the sequencing procedure 400. For example, the at least one processor 130 may determine whether enough detection results have been recorded to enable the at least one processor 130 (or some other processing entity, such as an external processor) to call a target number of bases (e.g., 150 bases). If not, the sequencing procedure 400 returns to step 404. If so, the sequencing procedure 400 ends at 432. Again, as explained above, the order in which the nucleotides are tested is arbitrary.

Mitigating FNI and FLR Errors

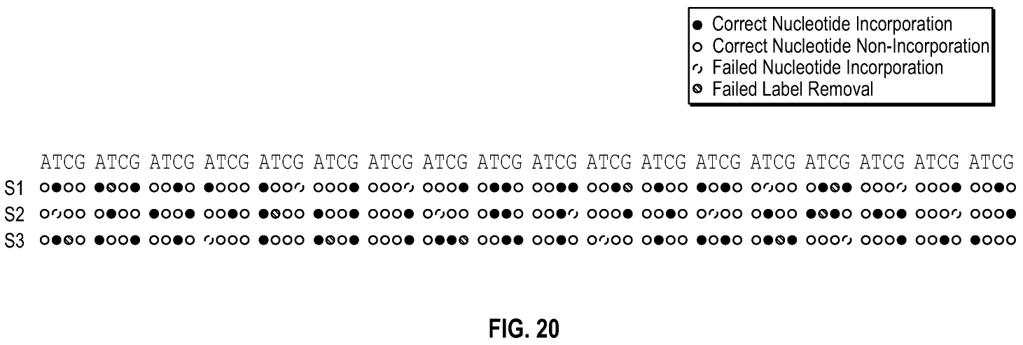

To illustrate the effects of FNI and FLR errors on CLUS devices and SMAS devices 100, each type of sequencer is used to call an exemplary DNA sequence with FNI and FLR errors occurring randomly as the sequence is read using the modified additive approach of SBS described above. The error rate is assumed to be r≅0.1 for both FNI and FLR errors. The exemplary sequence is: TAG CAA GGT CCG CTA CTG GCA GAC TGG. FIG. 20 shows both types of errors made at r≅0.1 throughout 18 inquiry cycles of A?⇒T?⇒C?⇒G? inquiry steps. As shown in FIG. 20, approximately 1 out of 10 reactions fail, and errors are evenly distributed between FNI errors and FLR errors for the three ssDNA instances being sequenced. The model case represents one of many possible scenarios of the ensemble behavior. Consequences of FNI and FLR errors on the base-calling precision are analyzed for the case when the three DNA strands are placed on a single sensor of a CLUS device and when they are placed on three discrete nanoscale sensors 105 of a SMAS device 100.

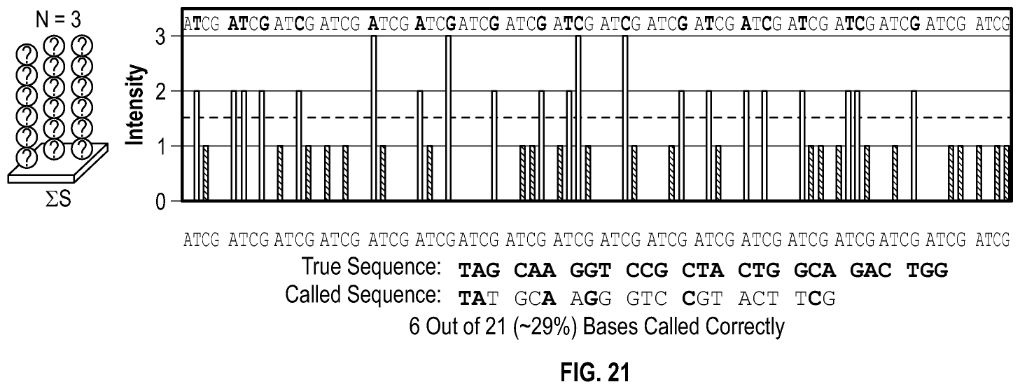

FIG. 21 illustrates the expected signal level detected by a CLUS device sensor capturing the behavior of the molecular ensemble during the sequencing procedure. At each inquiry step, the CLUS device sensor can detect four signal intensity levels of the molecular ensemble (made up of the three ssDNA): namely 0 labels, 1 label, 2 labels, or 3 labels detected. The sequencing procedure for a CLUS device considers the combined signal of the ensemble and cannot distinguish when reactions on individual strands are failing. A base is called at a particular inquiry step whenever the CLUS device sensor senses at least two labels. This threshold can be represented by a decision criterion: a base is called when the CLUS sensor signal level is greater than 1.5. As FIG. 21 indicates, the large rate of chemistry failures results in significant base-calling errors and very low base-calling precision. The CLUS device approach results in only 6 out of 21 (approximately 29%) called bases being in accordance with the true sequence. This level of accuracy is only slightly better than random guessing, which has 25% accuracy (because with 4 bases, there is 1 in 4 chance a base is guessed correctly). Moreover, CLUS devices cannot tell the difference between successful and failed chemistry reactions, nor do they know the positions of the FNI (dashed circles) or FLR (circles with backward-slash fill) errors shown in FIG. 20. For CLUS devices, the exact positions of FLR errors are obscured by ensemble averaging. Only probabilistic error correction algorithms can be implemented to marginally improve the quality of base-calling of a CLUS device by essentially making educated guesses about the positions of base insertion, deletion, and substitution sites. Exemplary algorithms are described in, e.g., A. Cacho et al., “A Comparison of Base-calling Algorithms for Illumina Sequencing Technology,” Briefings in Bioinformatics, Vol. 17(5), 786-795, 2016; W. C. Kao et al., “BayesCall: A model-based base-calling algorithm for high-throughput short-read sequencing,” Genome Res., Vol. 19(10), 1884-1895, 2009; and C. Ledergerber and C. Dessimoz, “Base-calling for next-generation sequencing platforms,” Brief Bioinform., Vol. 12, 489-97, 2011.

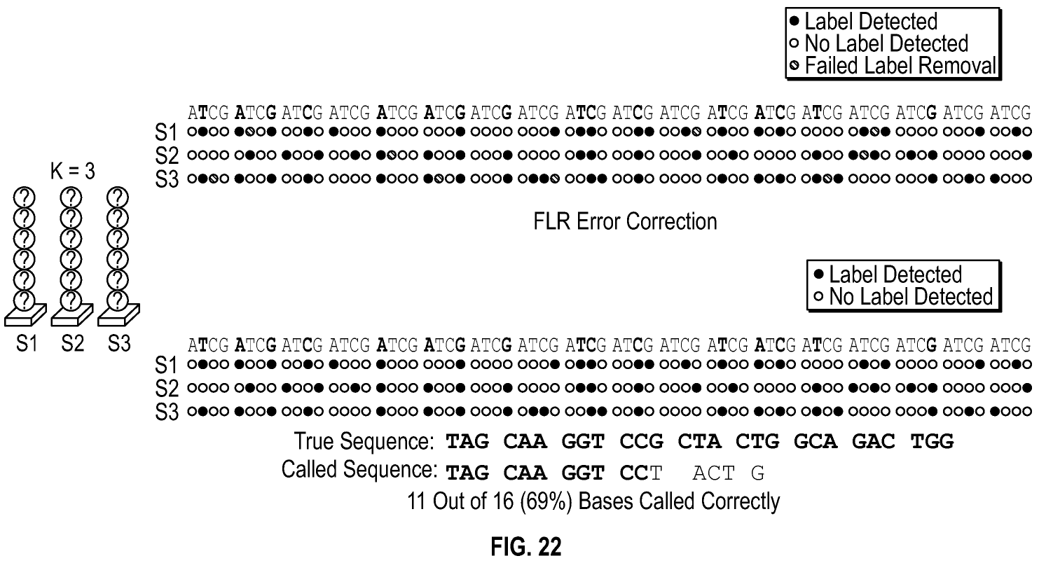

FIG. 22 illustrates how SMAS devices 100 can provide better accuracy when using the error-correction techniques described herein. As explained above, FLR errors occurring during the sequencing procedure can be detected during the sequencing procedure. Specifically, the SMAS device 100 knows (or can find) the positions of FLRs because the characteristic of each sensor 105 (e.g., signal level) is obtained and recorded after labels are cleaved and rinsed away and before the next nucleotide is introduced. The FLR errors can be corrected by treating them as “No Label Detected” when making base-calls. In other words, if a record of the sequencing procedure contains binary (e.g., 0/1) entries for each inquiry step, the FLRs can be corrected by changing the values at those inquiry steps from the “detected” value to the “not detected” value. As a specific example, if 0 represents no label detected and 1 represents a label detected, before error correction, a FLR at the mth inquiry step would be represented by a 1 in the mth position in a record. That error could be corrected by changing the value of 1 at the mth position in the record to a value of 0. The top portion of FIG. 22 illustrates the detection results for each of three sensors 105 of a SMAS device 100 before error correction to remove FLR errors. The lower portion of FIG. 22 shows the result of correcting the FLR errors before calling the bases.

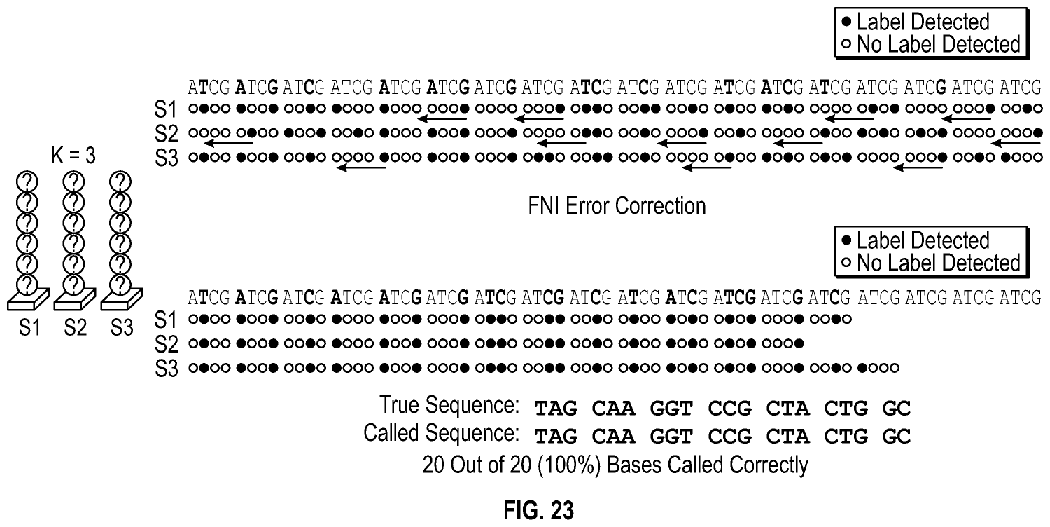

The modified additive sequencing procedure using a SMAS device 100 allows a base to be called for a particular inquiry step when more than half of the K sensors 105 (in the example of K=3, either two or three sensors 105) detect a label during that inquiry step. Unlike the CLUS device, however, a SMAS device 100 collects considerably more information because it detects the presence or absence of a label at every binding site 116 of a plurality (assumed in the example to be 3) of binding sites 116 and at every inquiry step of the sequencing procedure. As a result, using a SMAS device 100 can result in fewer base-calls being made, but those calls result in an estimated sequence that is considerably more accurate than the one called by a CLUS device. Specifically, for the exemplary sequence, once FLR errors have been removed (as shown in the lower portion of FIG. 22) use of the SMAS device 100 results in 11 out of 16 (approximately 69%) called bases being in accordance with the true sequence. Thus, FIGS. 21 and 22 illustrate that the consequences of chemistry failures on base-calling accuracy are considerably different for the two types of sequencing devices, and the SMAS device 100 provides better accuracy. When using a SMAS device 100, FNI errors can also be corrected because failed incorporations create a characteristic signature in the SMAS sensor 105 detection results (e.g., in a record made of label detections/non-detections by a sensor 105 during the sequencing procedure). In particular, FNI errors in the modified additive approach result in a run (a consecutive sequence) of zeros (or other “No-Label-Detected” detection results) for four or more consecutive inquiry steps. As explained in the discussion of FIG. 19, some FNI errors can be detected by identifying that a particular sensor 105 did not detect any label during an inquiry cycle. It is to be understood that FNI errors can also “span” multiple inquiry cycles. For example, assume that during a first inquiry cycle with A?⇒T?⇒C?⇒G? inquiry steps, a particular sensor 105 detects a label during the A? inquiry step, and then it does not detect any labels until the C? inquiry step of the next inquiry cycle. Because the C? inquiry step follows the A? inquiry step in the exemplary inquiry cycle, and the modified additive approach is being used as the sequencing cycle, the C? inquiry step of the first inquiry cycle should have resulted in detection of a label. Note that step 428 of FIG. 19 would not result in any FNI error being detected during either the first inquiry cycle or the second inquiry cycle because neither inquiry cycle resulted in no label being detected by the particular sensor 105. But an inspection of the record of detection results would reveal the presence of a FNI error. FNI errors can be corrected deterministically by deleting runs of (in the case of DNA sequencing, four) zeros to align the rogue strand with the strands unaffected by FNI errors. FIG. 23 illustrates the correction of FNI errors by deleting runs of four “no label detected” entries in records of detection results from the sequencing procedure. As shown in FIG. 23, FNI error correction results in a perfect alignment between the called and the true sequences.

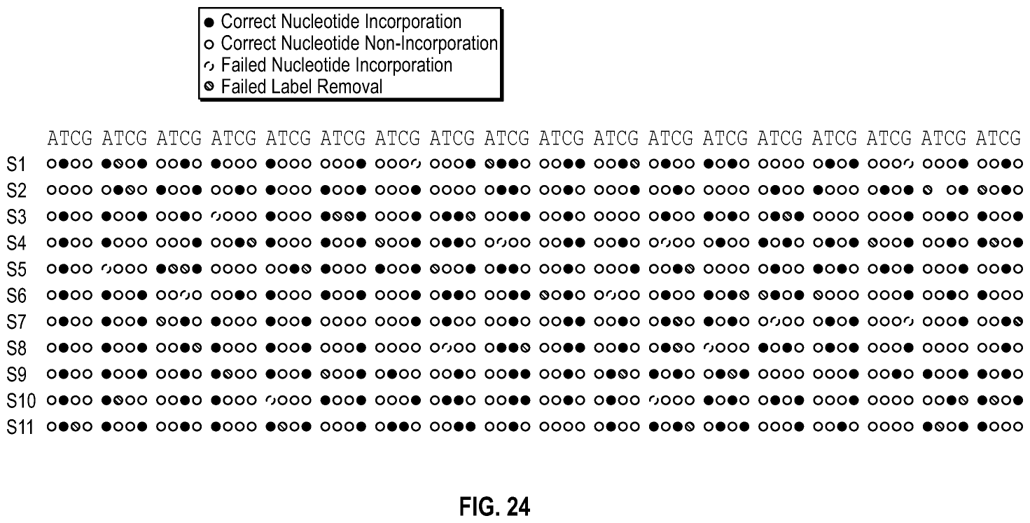

The qualitative analysis of the simplified model systems with a limited set of errors suggests that use of a SMAS device 100 for nucleic acid sequencing is vastly superior to use of a CLUS device, at least when the number of instances K of the sequenced DNA strand is small and chemistry failure rates are high. To set the framework for a quantitative comparison of the two platforms, how the cluster size (for a CLUS device) and the number of instances sequenced (for a SMAS device 100) affects the base-calling precision is explored below. Consider the case where N=K=11 and r=0.1 for both FNI and FLR errors. Assume the sensors are reading the same example sequence considered above (TAG CAA GGT CCG CTA CTG GCA GAC TGG) and that chemistry errors causing FNIs and FLRs occur randomly for 18 inquiry cycles of A?⇒T?⇒C?⇒G? inquiry steps. FIG. 24 illustrates the results of exemplary SBS reactions on 11 instances of a DNA strand with a large chemistry failure rate (r≅0.1 or 10%). As shown in FIG. 24, approximately 1 out of 10 reactions fails.

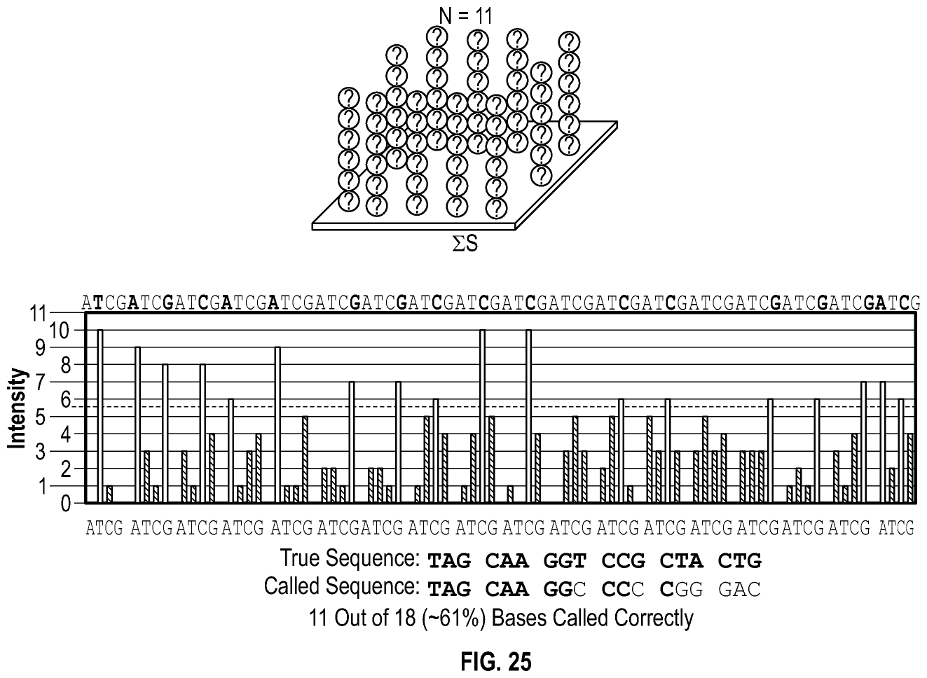

FIG. 25 illustrates the effect of the larger cluster size N on the base-calling accuracy of the CLUS device. FIG. 25 shows the expected signal level detected by a CLUS device sensor capturing the behavior of the molecular ensemble during the sequencing procedure. At each inquiry step, the CLUS device sensor can detect any one of twelve signal intensity levels of the molecular ensemble (the eleven ssDNA), namely, from 0 to 11 labels detected. A base is called at a particular inquiry step when the signal level detected by the CLUS sensor is greater than 5.5. As FIG. 25 shows, failed chemistry results in base-calling errors: only 11 out of 18 (approximately 61%) of called bases are in accordance with the true sequence.

A comparison of FIG. 25 with FIG. 21 indicates that the accuracy of the CLUS device with N=11 is better than when N=3. Specifically, increasing the cluster size N results in a considerable reduction in base-calling errors. Whereas in the N=3 case only about 29% of called bases were in agreement with the true sequence, increasing the cluster size to N=11 brings the agreement to about 61% because the CLUS device benefits from the collective behavior of a larger ensemble. State-of-the-art commercial CLUS-type sequencers work with arrays of clusters holding approximately 100 instances of DNA strands.

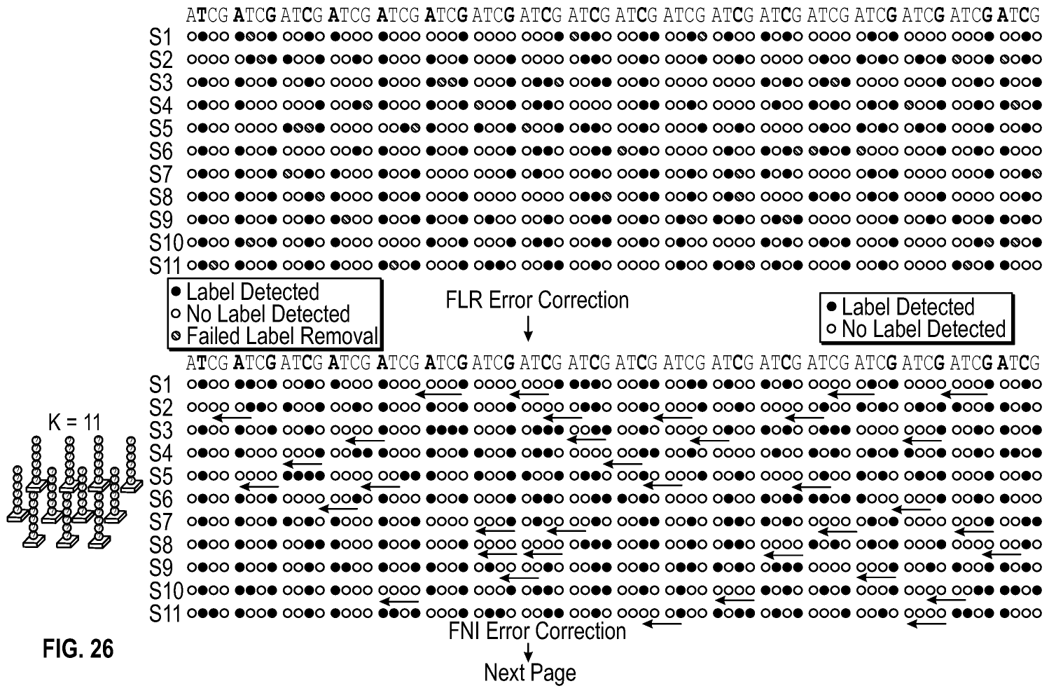

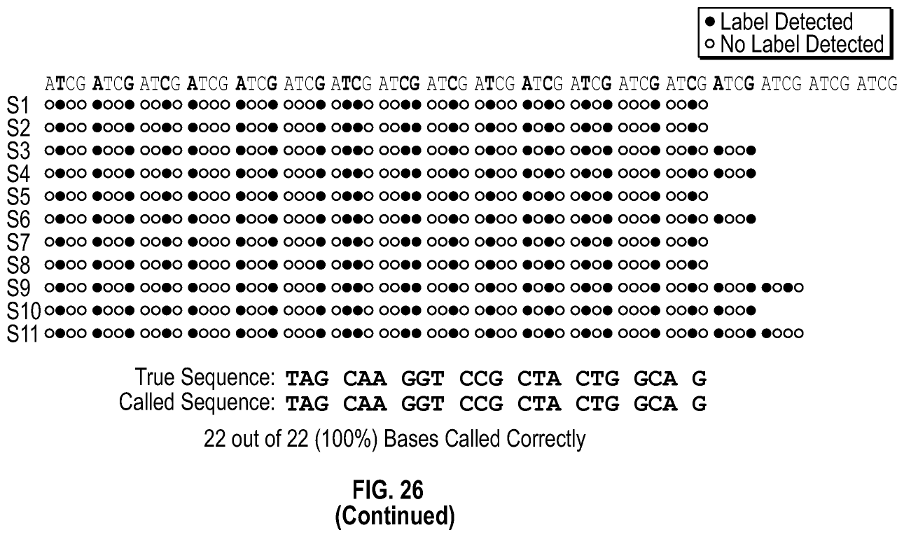

FIG. 26 illustrates the results when using a SMAS device 100 with K=11 (in other words, 11 instances of a ssDNA, each sensed by a different sensor 105) and deterministic error correction of FLR and FNI errors in accordance with some embodiments. A base is called at a particular inquiry step when more than half (e.g., at least 6 for K=11) of the sensors 105 detect a label. As shown by FIG. 26, implementing deterministic FLR error correction (middle) and FNI error correction (lower) as described above results in perfect alignment between the called and true sequences. Note that if no error detection/correction is performed, the called sequence based on data from a SMAS device 100 would be the same as that called using data from a CLUS device because a SMAS device 100 without error correction simply recreates the ensemble result by adding up all the individual sensor results. It is the ability to detect and correct errors in sequencing data that gives SMAS devices 100 an advantage relative to CLUS devices.

Thus, the use of a SMAS device 100 along with deterministic error correction can result in perfect agreement between the true and called sequences if only FNI and FLR errors occur. In addition, if only FNI and FLR errors occur, it is actually possible to call an error-free sequence using only a single sensor 105, reading a single ssDNA, along with the deterministic error correction techniques discussed above (e.g., changing FLRs to “no label detected” and/or deleting runs of “no label detected” of a specified length (e.g., 4) from the record of detection results).

When FNR and/or FDL errors are introduced, however, using only deterministic error-correction is unlikely, in general, to eliminate all errors in the records of detection results. To address FNR and/or FDL errors, probabilistic error-correction can be included either in addition to or instead of deterministic error-correction.

Mitigating FNI, FLR, and FNR Errors

This section further includes FNR errors in the analysis. The impact of such errors on a CLUS device's base-calling accuracy is equivalent to that of FNIs and FLRs because of the averaging that is inherent in a CLUS device's detection of labels in a cluster of instances of nucleic acid. FNR errors are considerably more detrimental to the performance of a sequencing methodology using a SMAS device 100 because the FNR errors cannot be corrected deterministically. (It should be noted that FNR errors cannot be corrected at all, per se, in CLUS devices. Instead, CLUS devices rely on ensemble behavior to mitigate the effects of FLR and other types of errors.)

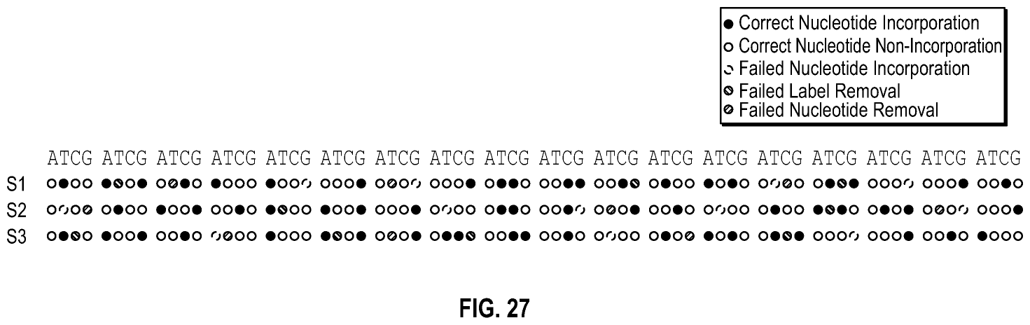

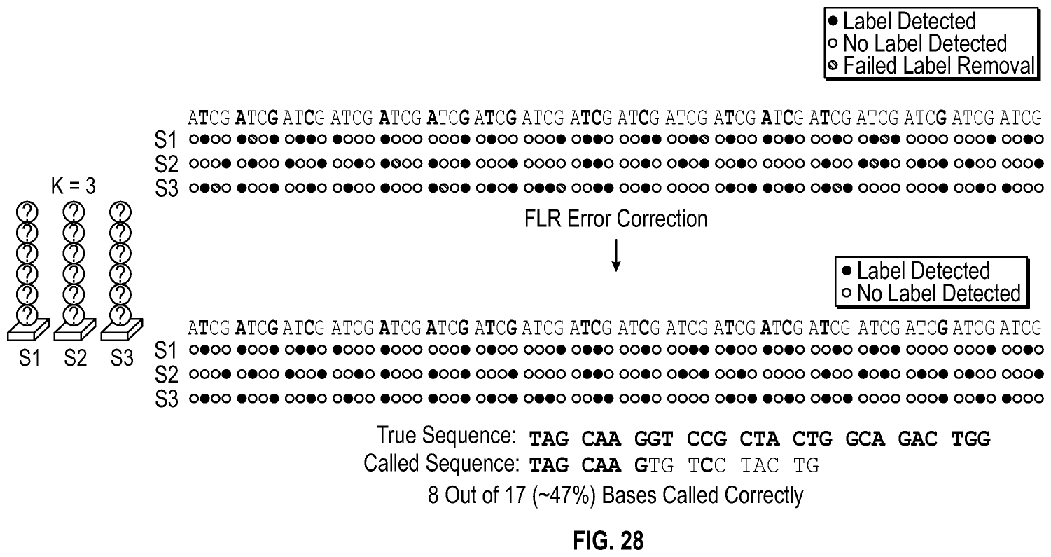

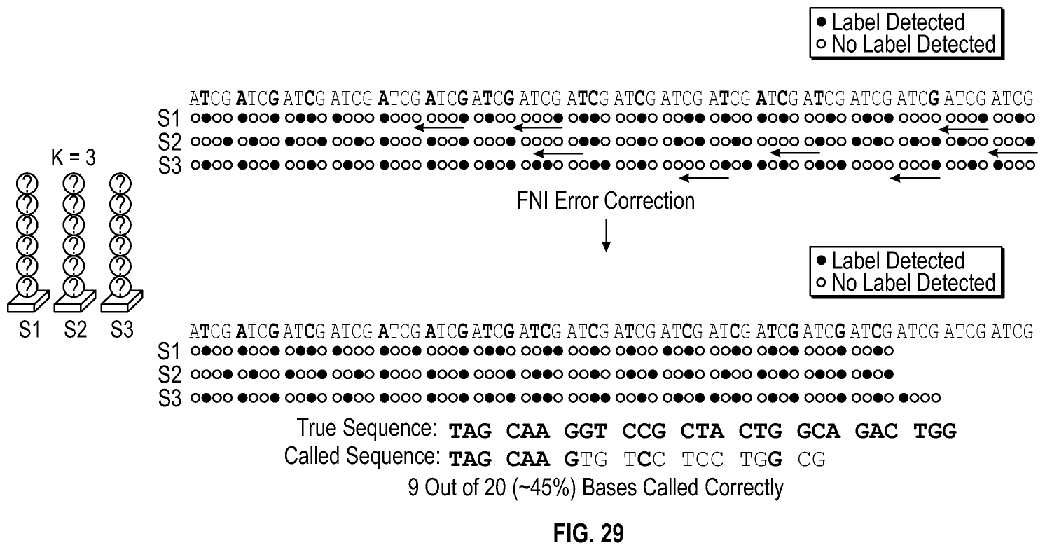

FIG. 27 illustrates the problem introduced by FNR errors in the exemplary sequence (TAG CAA GGT CCG CTA CTG GCA GAC TGG) assuming that FNI, FLR, and now also FNR errors occur randomly during 18 inquiry cycles of A?⇒T?⇒C?⇒G? inquiry steps. For purposes of example, assume K=3 (i.e., each of three binding sites 116 holds an instance of a particular ssDNA, and each of three respective sensors 105 senses a respective one of the three ssDNA instances), that 15 out of 100 reactions fail on average (r=0.15, which is a large chemistry failure rate), and that the errors are evenly distributed between FNI errors, FLR errors, and FNR errors. Under the example conditions and assumptions made here, simply given the data record created by SBS using a SMAS device 100, it is not possible to distinguish in the data record between correct detection events (solid circles in FIG. 27) and FNRs (circles with forward-slash fill). FIG. 28 illustrates the results when the base is called if more than half (at least 2 out of 3) of the sensors S1, S2, S3 detect a label. Although the FLR errors can be corrected deterministically (by treating them as “no label detected” as described above), the FNR errors cannot be identified because they are indistinguishable from correct label detection events. As a result, in this example, only 8 out of 17 (about 47%) of the called bases are in accordance with the true sequence. Thus, the introduction of FNR errors makes deterministic FNI error correction more challenging because FNR errors break the run of four or more “no label detected” detection results that could otherwise have been removed. If one naïvely implements FNI error correction by deleting runs of four zeros to attempt to align rogue strands with the strands unaffected by the error, the sequencing precision does not improve. Indeed, as shown in FIG. 29, for this example, the base-calling precision is seemingly made worse because after the runs of four “no label detected” detection results are removed, only 9 out of 20 (45%) of the base-calls are in agreement with the true sequence.

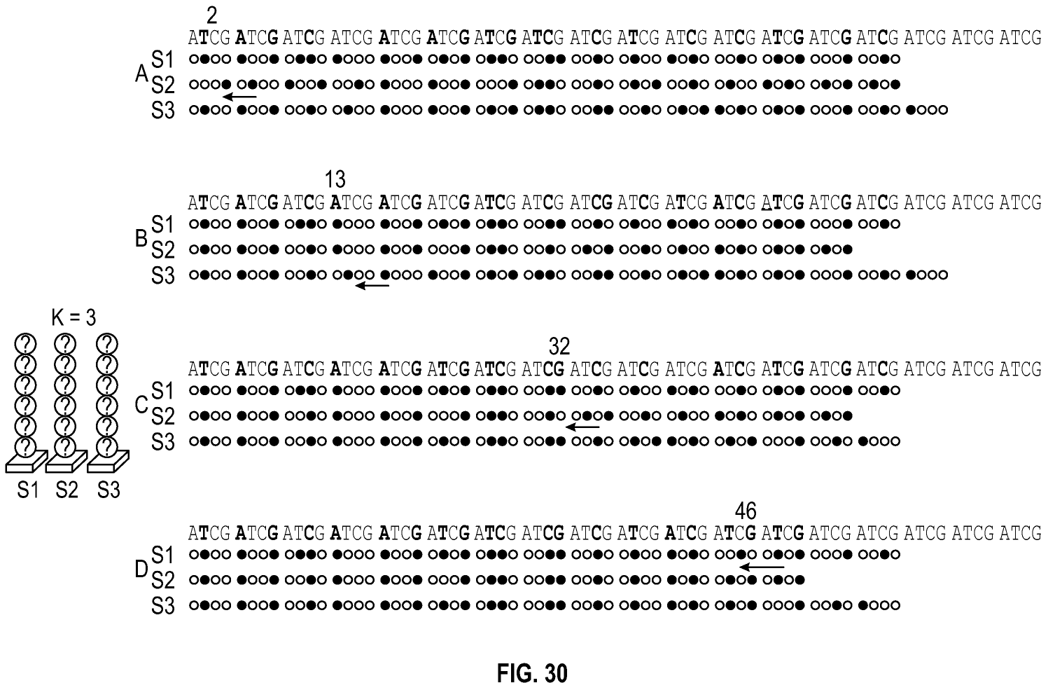

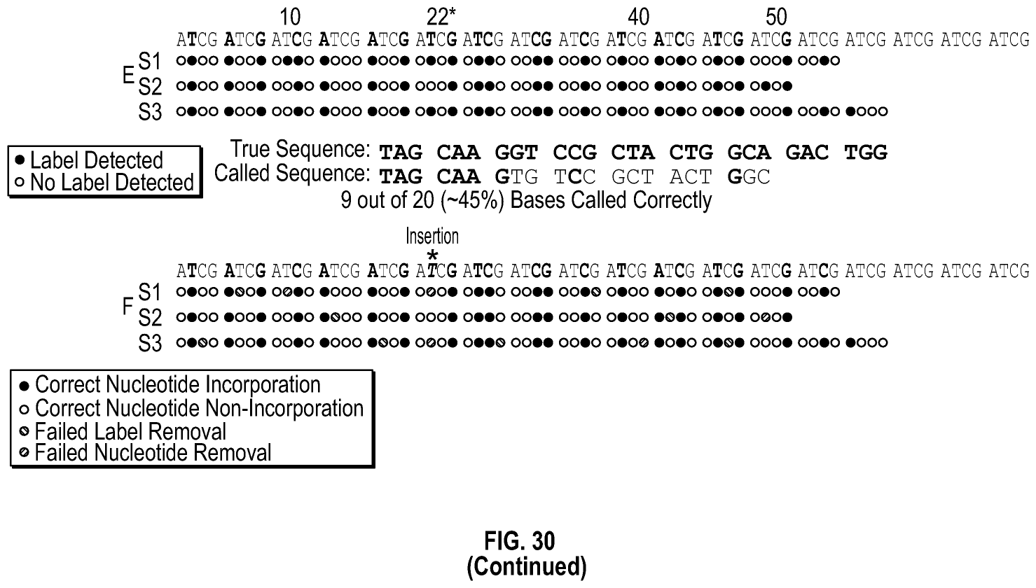

The error correction can be improved to mitigate FNR errors in addition to FLR and FNI errors by applying probabilistic error correction. For example, note the thymine-inquiry step at position 2 (inquiry step 2 of inquiry cycle 1). Sensors S1 and S3 detect labels, but S2 does not. S2 does not detect a label either because FNR errors occurred at both of sensors S1 and S3 simultaneously, or because a FNI error occurred at sensor S2. Assuming the probability of each error is r, the probability that FNR errors occurred simultaneously at both sensors S1 and S3 is r², and the probability of a FNI error at sensor S2 is r. The error correction algorithm (performed, e.g., by the at least one processor 130 or another processor) assumes the more likely event happened (there was a FNI error at sensor S2) and deletes, from the data record capturing the detection results from sensor S2, all entries in positions 2 to 5 to shift the S2 detection results in the S2 record. As a result, the detection results in the S2 record are realigned with the detection results produced by sensors S1 and S3, as shown in the upper portion of FIG. 30 labeled “A.” The G-label detection formerly (pre-deletion) at position 4 (in the portion of FIG. 30 labeled “A”) can now be attributed to FNR because sensors S1 and S3 do not detect labels in position 4 (inquiry step 4 of inquiry cycle 1).

The same error-correction procedure can be performed from left to right at positions 13 (as shown in the portion of FIG. 30 labeled “B”), 32 (labeled “C”) and 46 (labeled “D”) to show gradual improvement of alignment between the S1, S2, and S3 records of detection results, as illustrated in the portion of FIG. 30 labeled “E”. The portion of FIG. 30 labeled “E” indicates that although the implementation of multiple probabilistic error-correction steps aligns the outputs of all the sensors S1, S2, and S3, it does not seem to improve the alignment between the called and true sequences. Even after error correction, only 9 out of 20 (45%) of the bases are called correctly. In other words, base-call errors still occur. Specifically, following the error-correction procedure, all three sensors S1, S2, and S3 report having detected labels at the inquiry steps where labels should be detected, but some of the sensors also detect labels incorporated incorrectly by FNR at positions 10, 22, 40 and 50 (shown in the continuation view of FIG. 30).

Calling the base when more than half of the sensors 105 agree in their detection results (following error correction) results in a thymine insertion error at sequence position 8 (inquiry step 22), where sensors S1 and S3 both detect labels bound to non-complementary nucleotides during the same inquiry step. (It is to be understood that the reason it is possible to know there is a thymine insertion error at position 8 is because the errored data was created for purposes of illustration and is known. In an implementation, the sensors 105 merely indicate whether a label was detected during an inquiry step, not whether that detection (or lack of detection) was correct or in error. Thus, in an implementation, the errors at inquiry step 22 would be essentially indistinguishable from correct detection results.) The properly aligned true and called sequences, clearly displaying the position of single errant base insertion, can be presented as:

| Error: | \|Insertion |
|---|---|
| True Sequence: | TAG CAA G*G TCC GCT ACT GGC |
| Called Sequence: | TAG CAA GTG TCC GCT ACT GGC |

*insertion position

This insertion error can by corrected if the base-calling rule is modified to require all three sensors S1, S2, and S3 to be in agreement. With such a rule, all three sensors S1, S2, and S3 would have to suffer a FNR error simultaneously to cause a wrong base-call. The probability of such an event is only r³. Assuming that r=0.05, all three sensors S1, S2, and S3 suffer a FNR event during the same inquiry step on average only 125 in 100,000 inquiries (or a probability of 0.000125), which is extremely low even for the very high error rate used in the current example. Implementing such a rule could, however, result in incorrect calls if FLD errors are also occurring, as discussed further below.

Mitigating FNI, FLR, FNR, and FLD Errors

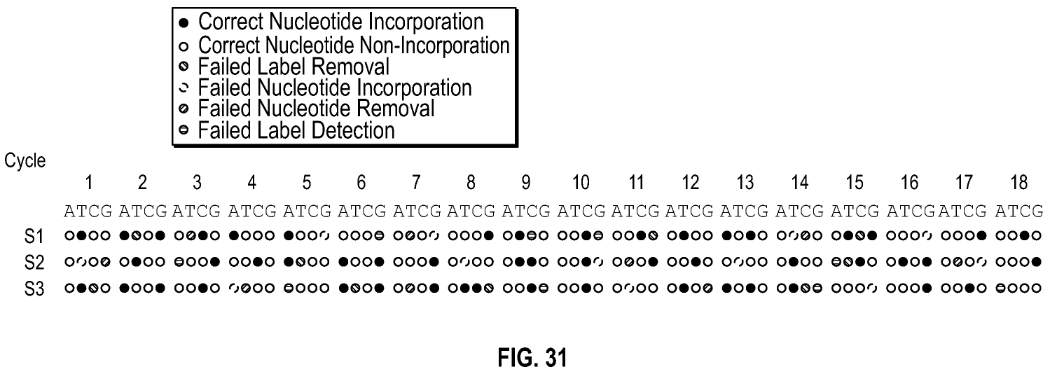

The general error-correction strategy used in some embodiments accounts for and mitigates all four types of chemistry failures causing FNI, FLR, FNR, and FLD errors. FIG. 31 illustrates the exemplary sequence (TAG CAA GGT CCG CTA CTG GCA GAC TGG) assuming that FNI, FLR, FNR, and now also FLD errors occur randomly during 18 inquiry cycles of A?⇒T?⇒C?⇒G? inquiry steps. For purposes of creating many errors in the sequencing data to provide a vehicle to illustrate exemplary error-correction procedures, assume a very high average error rate of 1 failed reaction out of 5 (r≅0.2, or a 20% error rate), and also assume the errors are evenly distributed between FNI errors, FLR errors, FNR errors, and FLD errors. Thus, approximately 20 out of 100 reactions fail, and the failures are equally distributed between the four error types. It will be appreciated that such a high error rate is unlikely to occur in practice, and therefore the difficulty of the example considered here is likely much higher than would be encountered in a real-world implementation.

Under the example conditions and assumptions made here, simply given the data record created by SBS using a SMAS device 100, it is not possible to distinguish between correct nucleotide incorporations and FNRs, nor between correct nucleotide non-incorporations and FNIs. Although the FLR errors can be detected and corrected deterministically as described previously (by checking the sensors 105 after cleaving and rinsing away labels, and treating FLRs as “no label detected”), the FNR errors cannot be identified because they are indistinguishable from correct detection events, and the FNI and FLD errors cannot be identified because they are indistinguishable from correct nucleotide non-incorporations. Nevertheless, error mitigation can still be accomplished using probabilistic error-correction techniques. For example, as explained above, when fewer than all of the sensors S1, S2, and S3 either detect or do not detect labels during a particular inquiry step, the probabilities of two (or more) events can be computed, the event having the highest probability can be assumed to be the correct one, and the appropriate error-correction step can be taken.

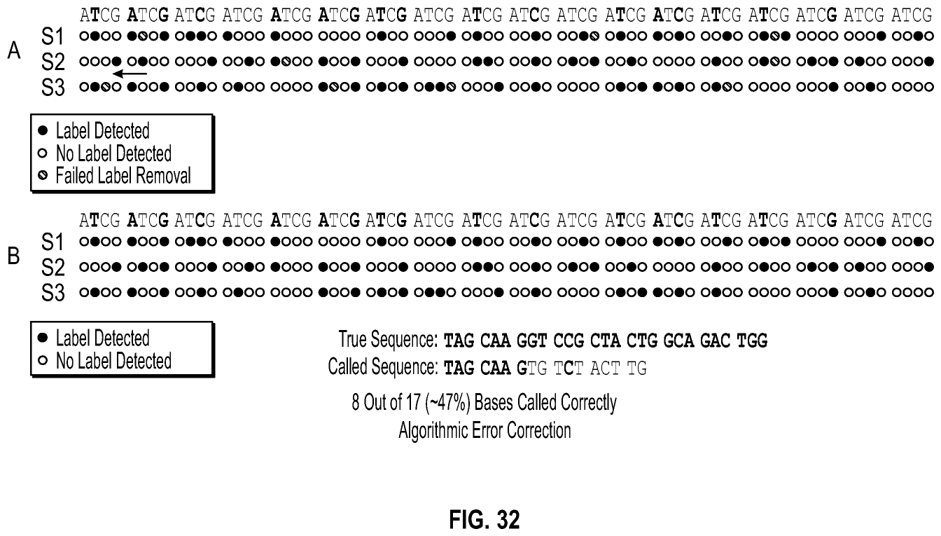

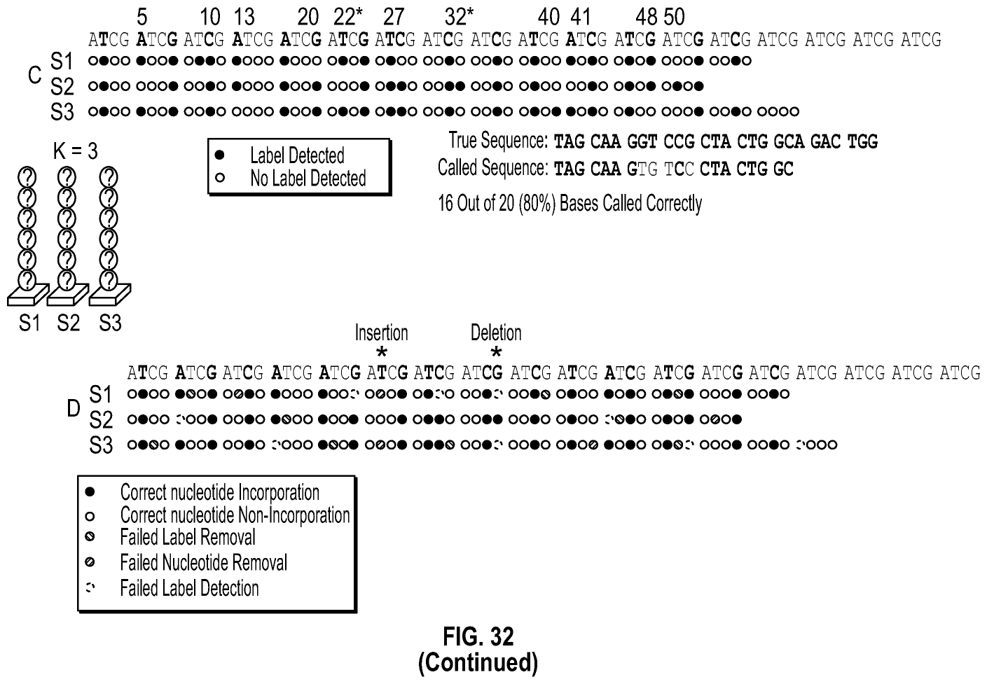

FIG. 32 illustrates the application of error-correction procedures to the data captured during SBS under the conditions and assumptions described above. The portion of FIG. 32 labeled “A” is the raw data before removal of FLR errors. Assuming, as described above, the sensor 105 signal levels are checked after labels are cleaved and rinsed away, the locations of FLR errors are known. The FLR errors can be eliminated altogether using deterministic error correction, namely by changing the “label detected” value (e.g., 1 or “yes”) to the “no label detected” (e.g., 0 or “no”) value in the data record in the positions corresponding to the inquiry steps where FLR errors were detected. Note that during the inquiry cycle 15 shown in FIG. 31, a FLR error follows a FLD error in the data for sensor S2. In other words, sensor S2 failed to detect the label of the incorporated nucleotide during the first inquiry step of the 15th inquiry cycle. When the labels are cleaved after the first inquiry step of the 15th cycle, and before the second inquiry step of the 15th inquiry cycle, the signal level of sensor S2 is checked. This check reveals the presence of a label at sensor S2, which would be known to be a FLR error because all labels should have been cleaved and rinsed away after the last inquiry step. Thus, even when a FLR error follows another error, it is detectable and can be removed.

The portion of FIG. 32 labeled “B” shows the records of detection results after removal of FLR errors via deterministic error correction, applied as described previously. The data records shown in “B” now contain only indications that a label was detected or not detected by each of the sensors S1, S2, S3 at each of the (4×18) inquiry steps shown. (It will be appreciated that the records can be shorter or longer than shown in FIG. 32.) As explained above, it is not known from these records which “label detected” indications are correct and which are FNR errors, and it is not known which “no label detected” indications are correct and which are FNI or FLD errors. As a result, probabilistic error correction can be used to estimate the sequence.

To explain how probabilistic error correction can be applied, the table below shows the data record of FIG. 32 for the first five inquiry cycles (inquiry steps 1-20) of the three sensors S1, S2, and S3 after FLR errors have been removed (e.g. from the records labeled “B” in FIG. 32). In other words, the table below shows the first 20 detection results following deterministic error correction to remove FLR errors. For inquiry steps during which a sensor detected a label, the table contains a value of 1, and for inquiry cycles during which a sensor did not detect a label, the table contains a value of 0:

| | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| Step | A | T | C | G | A | T | C | G | A | T | C | G | A | T | C | G | A | T | C | G |
| S1 | 0 | 1 | 0 | 0 | 1 | 0 | 0 | 1 | 0 | 1 | 1 | 0 | 1 | 0 | 0 | 0 | 1 | 0 | 0 | 0 |
| S2 | 0 | 0 | 0 | 1 | 0 | 1 | 0 | 0 | 0 | 0 | 0 | 1 | 0 | 0 | 1 | 0 | 1 | 0 | 0 | 0 |
| S3 | 0 | 1 | 0 | 0 | 1 | 0 | 0 | 1 | 0 | 0 | 1 | 0 | 0 | 1 | 0 | 0 | 0 | 0 | 0 | 0 |

As explained above, a simple majority vote after removal of FLR errors would result in only 8 of the 17 bases being called correctly, as shown in the portion of FIG. 32 labeled “B.” Probabilistic error correction, as described below, can provide a significant improvement.

Considering inquiry step 2 as an example, both of sensors S1 and S3 detected labels (entries in the table above are 1s), but sensor S2 did not (table entry is 0). Thus, either both sensors S1 and S3 are wrong, or sensor S2 is wrong. By taking into account the probabilities of the various events that could lead to each of these outcomes, the error correction algorithm can mitigate errors in the sequencing data. Specifically, because FLRs have been removed from the data record, the only way both sensors S1 and S3 incorrectly detected labels during inquiry step 2 is if both suffered FNR errors during that inquiry step. If the probability of a FNR error is r, then the probability that both sensors S1 and S3 suffer FNR errors during a single inquiry step is r². For purposes of this example, a high error rate of r=0.2 is assumed, and therefore the probability that both sensors S1 and S3 incorrectly detected labels during inquiry step 2 is 0.04.

If sensor S2 is wrong, it is because sensor S2 failed to detect a label due to either a FLD error or a FNI error. Recall that a FLD error occurs when the correct complementary nucleotide is incorporated, but it is either missing a label or the sensor fails to detect its label, and a FNI error occurs when the correct complementary nucleotide is not incorporated at all during a sequencing cycle. FLD and FNI errors are mutually exclusive (i.e., a sensor can only suffer from one of them at a time, and never both). Therefore, assuming the probability of each type of error is r, the probability that sensor S2 suffered either a FLD error or a FNI error is 2r. For the example here, a high error rate of r=0.2 has been assumed, so the probability that sensor S2 is wrong during inquiry step 2 is 0.4. Comparing the probability that sensor S2 is wrong during inquiry step 2 to the probability that both of sensors S1 and S3 are wrong, because 0.4>>0.4, it is much more likely that sensor S2 is wrong. In some embodiments, the error-correction algorithm assumes that the more likely event occurred, meaning that sensor S2 is assumed to be wrong, and the possibility that both sensors S1 and S3 are wrong is discarded and not considered further.

As explained above, sensor S2 could be wrong because of either a FLD error or a FNI error. Following a FLD error, the DNA strand being sensed by sensor S2 would remain “in synch” or “aligned” with the DNA strands being sensed by sensors S1 and S3. In other words, if inquiry step m sequenced the base of the DNA strands being sensed by each of the sensors S1, S2, and S3, then inquiry step m+1 would sequence the 41st base of each strand, even if one of the sensors (e.g., sensor S2) suffered a FLD error during inquiry step m. On the other hand, a consequence of a FNI error is that the DNA strand being sensed by the sensor that suffers a FNI error goes “out of synch” or becomes “misaligned” with the DNA strands being sensed by sensors that did not suffer from FNI errors. In the example at hand, the DNA strand being sensed by sensor S2 would become out of synch with the DNA strands being sensed by sensors S1 and S3 if the error at inquiry step 2 were due to a FNI (e.g., it would be “behind” the DNA strands being sensed by sensors S1 and S3 by four inquiry steps, which would be the next time the complementary nucleotide could be incorporated).

In some embodiments, the action taken by the error-correction algorithm depends in part on an inspection of candidate error-corrected data that separately assumes each of the two types of error has occurred. In other words, the record of detection results can be modified to correct the error assuming it was caused by a FLD error to produce a first candidate corrected data record, and the record of detection results can be separately modified to correct the error assuming it was caused by a FNI error to produce a second candidate corrected data record. The two candidate corrected data records can then be inspected and/or analyzed and/or compared to determine which is more likely to be correct. To correct a FLD error, the “no label detected” indication is flipped to a “label detected” indication. To correct a FNI error, the data entries are shifted by four places (e.g., to the left as the data records are presented in the examples herein).

To illustrate for the specific example of inquiry step 2 in the example data record, a first candidate corrected data record, Option A, assumes that the (presumed) error affecting sensor S2's output was a FLD error. That presumed error is corrected by flipping the bit for inquiry step 2 in sensor S2's record from 0 to 1 as shown in the Option A table below by the boldface, underlined value “1”:

| | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| Step | A | T | C | G | A | T | C | G | A | T | C | G | A | T | C | G | A | T | C | G |
| S1 | 0 | 1 | 0 | 0 | 1 | 0 | 0 | 1 | 0 | 1 | 1 | 0 | 1 | 0 | 0 | 0 | 1 | 0 | 0 | 0 |
| S2 | 0 | 1 | 0 | 1 | 0 | 1 | 0 | 0 | 0 | 0 | 0 | 1 | 0 | 0 | 1 | 0 | 1 | 0 | 0 | 0 |
| S3 | 0 | 1 | 0 | 0 | 1 | 0 | 0 | 1 | 0 | 0 | 1 | 0 | 0 | 1 | 0 | 0 | 0 | 0 | 0 | 0 |

The second candidate corrected data record, Option B, assumes that the error affecting sensor S2's output was a FNI error. That presumed error is corrected by deleting from the sensor S2 data entries the data recorded during inquiry steps 2, 3, 4, and 5 to “resynchronize” or “realign” the data record corresponding to sensor S2 with the data records of sensors S1 and S3, which results in the table below (shifting into places 17-20 the values formerly at places 21-24). The Option B table entries modified by the error-correction algorithm are shown in boldface, underlined type:

| | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| Step | A | T | C | G | A | T | C | G | A | T | C | G | A | T | C | G | A | T | C | G |
| S1 | 0 | 1 | 0 | 0 | 1 | 0 | 0 | 1 | 0 | 1 | 1 | 0 | 1 | 0 | 0 | 0 | 1 | 0 | 0 | 0 |
| S2 | 0 | 1 | 0 | 0 | 0 | 0 | 0 | 1 | 0 | 0 | 1 | 0 | 1 | 0 | 0 | 0 | 1 | 0 | 0 | 1 |
| S3 | 0 | 1 | 0 | 0 | 1 | 0 | 0 | 1 | 0 | 0 | 1 | 0 | 0 | 1 | 0 | 0 | 0 | 0 | 0 | 0 |

Options A and B can then be compared and/or analyzed to determine which is more likely to be correct, and it may be possible to discard one of the options. For example, a processor (e.g., the at least one processor 130 or another processor) can determine the value of a metric for each candidate corrected data record and decide, based at least in part on a comparison of the metrics, which of Options A and B is more likely to be correct. An example of a metric is the number of inquiry steps starting from the one after the now-corrected current inquiry step and the inquiry step J positions further away in the data record for which all three (or, more generally, K) sensors' label detection results agree. Using this metric, for example, and setting the value of J to 8, the value of the metric for Option A is 3, and for Option B it is 6. In some embodiments, based on this result only, it is assumed that because the value of the metric for Option B is significantly larger than the value of the metric for Option A, Option B is more likely to be correct, and Option A is discarded. In some embodiments, one of the two options is discarded only if the value of its metric exceeds the value of the other option's metric by some threshold (e.g., a percentage, an amount (e.g., at least double, at least 1.5 as large, etc.), etc.). In some embodiments, Option A is retained, and no options are discarded until later.

In some embodiments, contributions to the value of the metric are weighted based on the distance of the data being considered from the now-corrected current inquiry step. For example, because the likelihood of additional errors having been introduced in the data record increases as more bases are sequenced (e.g., the likelihood of some kind of error occurring for one of the K sensors between inquiry step 3 and inquiry step 40 is larger than the likelihood of some kind of error occurring for one of the K sensors between inquiry step 3 and inquiry step 6), the metric can assume that closer data entries are more likely to be correct than are further-away data entries, and, accordingly, give more weight to the data entries closer to the now-corrected data entry than to those further away. The weighting may be, for example, linear or nonlinear. As just one example, for a metric with contributions from data up to 12 inquiry steps away, contributions from inquiry steps within four inquiry steps of the now-corrected data may be given a weight of 1, contributions from inquiry steps between five and eight inquiry steps of the now-corrected data may be given a weight of 0.5, and contributions from inquiry steps between nine and twelve inquiry steps of the now-corrected data may be given a weight of 0.2. It is to be appreciated that many possible metrics, whether with or without weighting, can be used, and those provided above are merely exemplary and are not intended to be limiting.

It is also to be appreciated that although the metrics described above use the number of inquiry steps starting from the one after the now-corrected current inquiry step and the inquiry step J positions further away in the data record for which all three (or, more generally, K) sensors' label detection results agree, they could equivalently use the number of inquiry steps starting from the one after the now-corrected current inquiry step and the inquiry step J positions further away in the data record for which all three (or, more generally, K) sensors' label detection results do not agree. In this case, a large value of the metric would indicate more mismatches between sensor data entries, and therefore a candidate corrected data record would be more likely to be correct for lower values of the metric. Adjustments could be made to any weighting to be applied, as will be apparent to those having ordinary skill in the art.

It is also to be appreciated that it is not necessary to discard one of the possible options following correction of a presumed error in the data record. For example, following the (presumed) correction of the (presumed) error at inquiry step 2 in sensor S2's record, both of Options A and B can be retained, and further error detection and correction performed on both in parallel. Likewise, each time a presumed error is corrected, multiple options for candidate sequences can be determined and/or assessed/compared. A running metric value can be maintained for each possible option/candidate sequence at each step of the error-correction procedure, and the most likely candidate sequence can be determined at some point (e.g., after all candidate options have been determined and evaluated (e.g., relative to each other), or after some additional number of inquiry steps, etc.).

Moreover, although in the example above the possibility that both sensors S1 and S3 wrongly detected labels was discarded immediately because the probability of that event (given the assumptions herein) is significantly lower than the probability that sensor S2 was wrong, the same procedure as for sensor S2 could be followed instead. In other words, an Option C at inquiry step 2 could be determined assuming that both sensors S1 and S3 suffered FNR errors, and sensor S2 was correct. In this case, the metric can be adjusted to account for the likelihood of the various possible outcomes (e.g., by “penalizing” the metric of Option C based on the probability of sensors S1 and S3 both suffering FNR errors (e.g., multiplying the metric by the ratio of the probability of both sensors S1 and S3 being wrong to the probability of sensor S2 being wrong)).

It is to be appreciated that the error-correction methodologies described herein can be leveraged in a number of ways to improve the accuracy of nucleic acid sequencing using SMAS devices 100. Assuming sufficient computational power, it is possible for an implementation (e.g., using the at least one processor 130 or another processor or processors) to determine and evaluate an exhaustive set of candidate sequences with error-correction applied, and then choose the candidate sequence from among them that is most likely to be correct. To reduce computational complexity, it is also possible for an implementation to make decisions during the error-correction process to eliminate candidate error-corrected sequences (or potential error sources) that are deemed sufficiently unlikely to be correct (e.g., Option C in the example above) and to retain only those candidate error-corrected sequences that are more likely to be correct. It is to be appreciated that flexibility in the disclosed principles makes them suitable for error mitigation in systems having a wide variety of computational power.

Returning to the example above, assuming Option B was the only option retained after error correction was applied to the data from inquiry step 2, the corrected data appears below:

| | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| Step | A | T | C | G | A | T | C | G | A | T | C | G | A | T | C | G | A | T | C | G |
| S1 | 0 | 1 | 0 | 0 | 1 | 0 | 0 | 1 | 0 | 1 | 1 | 0 | 1 | 0 | 0 | 0 | 1 | 0 | 0 | 0 |
| S2 | 0 | 1 | 0 | 0 | 0 | 0 | 0 | 1 | 0 | 0 | 1 | 0 | 1 | 0 | 0 | 0 | 1 | 0 | 0 | 1 |
| S3 | 0 | 1 | 0 | 0 | 1 | 0 | 0 | 1 | 0 | 0 | 1 | 0 | 0 | 1 | 0 | 0 | 0 | 0 | 0 | 0 |

The next inquiry step where the three sensors S1, S2, and S3 do not agree is at inquiry step 5. Once again, sensor S2 does not agree with sensors S1 and S3 in the same manner as in inquiry step 2. In some embodiments, the error-correction algorithm determines that (a) the probability that sensor S2 is wrong is greater than the probability that both sensors S1 and S3 are wrong, and (b) sensor S2 suffered either a FNI error or a FLD error at inquiry step 5. Once again, two options may be created, one assuming the error was a FLD error (corrected by flipping the bit), and the other assuming the error was a FNI (corrected by shifting the data by four places). The corrected data records appear below:

Option A (presumed FLD error corrected):

| | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| Step | A | T | C | G | A | T | C | G | A | T | C | G | A | T | C | G | A | T | C | G |
| S1 | 0 | 1 | 0 | 0 | 1 | 0 | 0 | 1 | 0 | 1 | 1 | 0 | 1 | 0 | 0 | 0 | 1 | 0 | 0 | 0 |
| S2 | 0 | 1 | 0 | 0 | 1 | 0 | 0 | 1 | 0 | 0 | 1 | 0 | 1 | 0 | 0 | 0 | 1 | 0 | 0 | 1 |
| S3 | 0 | 1 | 0 | 0 | 1 | 0 | 0 | 1 | 0 | 0 | 1 | 0 | 0 | 1 | 0 | 0 | 0 | 0 | 0 | 0 |

Option B (presumed FNI error corrected):

| | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| Step | A | T | C | G | A | T | C | G | A | T | C | G | A | T | C | G | A | T | C | G |
| S1 | 0 | 1 | 0 | 0 | 1 | 0 | 0 | 1 | 0 | 1 | 1 | 0 | 1 | 0 | 0 | 0 | 1 | 0 | 0 | 0 |
| S2 | 0 | 1 | 0 | 0 | 0 | 0 | 1 | 0 | 1 | 0 | 0 | 0 | 1 | 0 | 0 | 1 | 0 | 0 | 0 | 1 |
| S3 | 0 | 1 | 0 | 0 | 1 | 0 | 0 | 1 | 0 | 0 | 1 | 0 | \| 0 | 1 | 0 | 0 | 0 | 0 | 0 | 0 |

Once again, metrics may be computed for Options A and B, and one of the options may be discarded, or both may be retained. For the sake of example, assume Option A is retained, resulting in the following error-corrected data:

| | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| Step | A | T | C | G | A | T | C | G | A | T | C | G | A | T | C | G | A | T | C | G |
| S1 | 0 | 1 | 0 | 0 | 1 | 0 | 0 | 1 | 0 | 1 | 1 | 0 | 1 | 0 | 0 | 0 | 1 | 0 | 0 | 0 |
| S2 | 0 | 1 | 0 | 0 | 1 | 0 | 0 | 1 | 0 | 0 | 1 | 0 | 1 | 0 | 0 | 0 | 1 | 0 | 0 | 1 |
| S3 | 0 | 1 | 0 | 0 | 1 | 0 | 0 | 1 | 0 | 0 | 1 | 0 | 1 | 0 | 1 | 0 | 0 | 0 | 0 | 0 |

The next inquiry step for which the sensors' data does not agree is inquiry step 10. Here, sensor S1 detected a label, but neither sensor S2 nor sensor S3 did. Because FLR errors have been removed from the data record, the only way sensor S1 incorrectly detected a label during inquiry step 10 is if it suffered a FNR error during that inquiry step. The probability of a FNR error is r. If sensors S2 and S3 are both wrong, it is because (a) both of them suffered FNI errors, (b) both of them suffered FLD errors, or (c) one of them suffered a FNI error and the other suffered a FLD error. The probability of any of events (a), (b), or (c), which are mutually exclusive, is 4r². Accordingly, in some embodiments, it is assumed that the more likely event happened, namely that sensor S1 suffered a FNR error (because r>>4r² for the assumed value of r). As explained above, FNR errors can be corrected by flipping the data entry from the “label detected” value to the “no label detected” value, which results in the following table:

| | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| Step | A | T | C | G | A | T | C | G | A | T | C | G | A | T | C | G | A | T | C | G |
| S1 | 0 | 1 | 0 | 0 | 1 | 0 | 0 | 1 | 0 | 0 | 1 | 0 | 1 | 0 | 0 | 0 | 1 | 0 | 0 | 0 |
| S2 | 0 | 1 | 0 | 0 | 1 | 0 | 0 | 1 | 0 | 0 | 1 | 0 | 1 | 0 | 0 | 0 | 1 | 0 | 0 | 1 |
| S3 | 0 | 1 | 0 | 0 | 1 | 0 | 0 | 1 | 0 | 0 | 1 | 0 | 0 | 1 | 0 | 0 | 0 | 0 | 0 | 0 |

The error-correction procedure can continue as described throughout the rest of the data record. The portion of FIG. 32 labeled “C” shows the results for the example. As indicated, following the application of the probabilistic error correction as described above, 16 out of 20 (80%) of bases are called correctly.

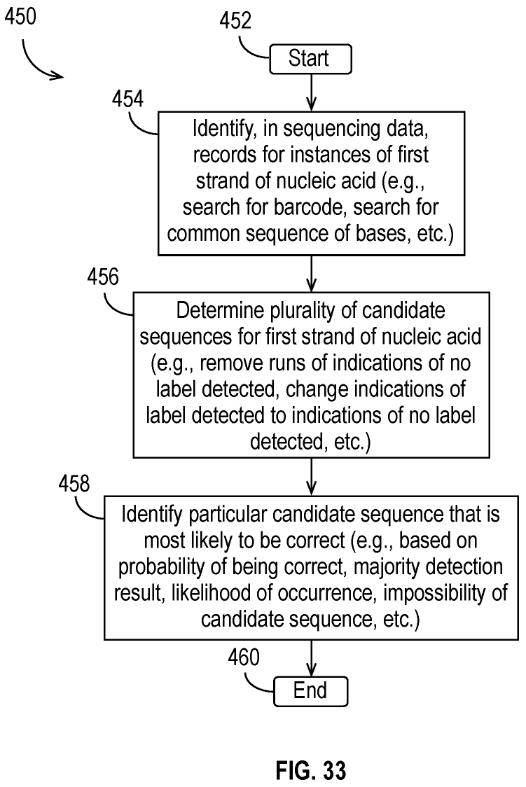

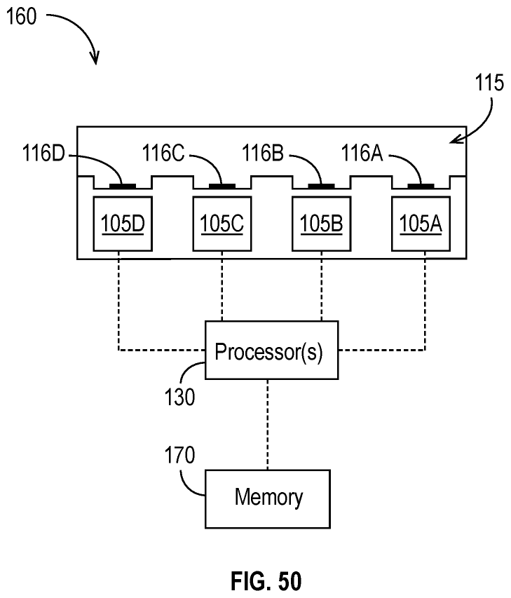

FIG. 33 is a flow diagram illustrating an error-correction procedure 450 in accordance with some embodiments. The error-correction procedure 450 may be, for example, the error-correction procedure 212 illustrated in FIG. 11, and it may be performed by a processor (e.g., the at least one processor 130 illustrated in FIG. 5A or in FIG. 50, discussed below). At 452, the error-correction procedure 450 starts. At 454, a plurality of records is identified in sequencing data generated as a result of a nucleic acid sequencing procedure that uses a SMAS device 100. Each of the identified plurality of records comprises a plurality of entries, each of which captures a detection result for one instance of a particular strand of nucleic acid. Thus, if the number of identified records is K, each of the K records contains one entry per detection result per inquiry step of the sequencing procedure. Each detection result indicates that, during the inquiry step, either (a) a label was detected by the corresponding sensor 105, or (b) no label was detected by the corresponding sensor 105. The plurality of records can be identified in a number of ways. For example, as explained further below, different unique barcodes can be ligated to the primer ends of nucleic acid strands so that a known sequence is read during the cycles of a sequencing procedure. Thus, the plurality of records can be identified by searching the sequencing data for a barcode associated with the particular strand of nucleic acid. As another example, a common sequence of entries can be identified in the sequencing data (e.g., within the entries documenting the detection results for the first approximately 35 inquiry steps of the sequencing procedure).

At 456, based on the plurality of records, a plurality of candidate sequences is determined for the particular strand of nucleic acid. Each of the plurality of candidate sequences estimates at least a portion (e.g., as little as one base) of the nucleic acid sequence of the particular strand of nucleic acid. In some embodiments, determining the plurality of candidate sequences comprises identifying within the plurality of records a particular inquiry step at which a first sensor detected a respective label and a second sensor did not detect any label, and establishing two candidate sequences, one of which assumes the first sensor correctly detected the respective label and the second of which assumes the first sensor incorrectly detected the respective label. In some embodiments, determining the plurality of candidate sequences comprises identifying within the plurality of records a particular inquiry step at which a first sensor detected a respective label and a second sensor did not detect any label, and establishing two candidate sequences, one of which assumes the second sensor incorrectly failed to detect any label and the second of which assumes the second sensor correctly failed to detect any label. In some embodiments, determining the plurality of candidate sequences comprises identifying, in at least one of the plurality of records, a set of consecutive entries (e.g., four entries) indicating that no label was detected, and deleting the set of consecutive entries indicating that no label was detected from the at least one of the plurality of records. In some embodiments, each of the plurality of entries is a first binary value (indicating that a label was detected) or a second binary value (indicating that no label was detected), and determining the plurality of candidate sequences comprises identifying, in at least one of the plurality of records, a run of (e.g., four) second binary values, and deleting the run of the second binary values from the at least one of the plurality of records.

At 458, a particular candidate sequence of the plurality of candidate nucleic acid sequences is identified as the sequence that is, from among the plurality of candidate sequences, most likely to be correct. In some embodiments, identifying the particular candidate sequence of the plurality of candidate sequences that is most likely to be correct comprises determining or estimating which of the plurality of candidate sequences has a highest probability of being correct. In some embodiments, identifying the particular candidate sequence of the plurality of candidate sequences that is most likely to be correct comprises determining, for each of the candidate sequences, a respective metric, and, based at least in part on the respective metrics and a criterion (e.g., a minimum likelihood of occurrence, a threshold likelihood of occurrence), choosing a particular candidate sequence as the one that is most likely to be correct. In some embodiments, identifying the particular candidate sequence of the plurality of candidate sequences that is most likely to be correct comprises identifying a majority result (e.g., either that more than half of the sensors 105 detected a label or that more than half of the sensors 105 did not detect a label) for a particular inquiry step represented by the plurality of records. In some embodiments, identifying the particular candidate sequence of the plurality of candidate sequences that is most likely to be correct comprises determining, for each of the plurality of candidate sequences, a respective likelihood of occurrence, and choosing the particular candidate sequence based on its respective likelihood of occurrence meeting a constraint (e.g., a minimum probability). In some embodiments, the particular candidate sequence that has the highest likelihood of occurrence among the candidate sequences is identified as the one most likely to be correct. In some embodiments, one or more of the candidate sequences are eliminated based on a known constraint, such as knowledge that a particular sequence of bases is impossible. For example, it may be known from the origin or source of the nucleic acid (e.g., a human being) that particular sequences of bases are impossible, and therefore candidate sequences that have such impossible sequences can be eliminated from further consideration.

At 460, the error-correction procedure 450 ends.

It should be understood that probabilistic error correction is successful only when the identified most-likely scenario (e.g., the identification at 458 of FIG. 33) is actually the correct one. If the chemistry failure rates are high, as in the examples described herein, there could be multiple scenarios that are equally likely to occur (or their probabilities of occurrence are close to each other), in which case more sophisticated bioinformatics tools may be employed. For example, a candidate sequence might be eliminated based on knowledge of the source of the nucleic acid being sequenced (e.g., based on knowledge that a particular sequence of bases is impossible given the source/origin of the nucleic acid). Nevertheless, if correctly implemented as described herein, the error correction process results in correct alignment of the sensor 105 outputs. In the example shown in FIG. 32, following removal of FNIs and FLRs, all three sensors S1, S2, and S3 report labels at the correct detection inquiry step where labels should be detected, but the sensors disagree at numerous inquiry positions (5, 10, 13, 20 22, 27, 32, 40, 41, 48 and 50) where sensors detect labels incorporated incorrectly by FNR or fail to detect labels due to FLD. Calling the base when more than half of the sensors 105 in the aligned sequence agree results in thymine insertion at sequence position 8 (inquiry step 22) and guanine deletion at position 13 (inquiry step 32). The properly aligned true and called sequences that clearly display base insertion and deletion positions can be presented as:

| Error: | Insertion Deletion |
|---|---|
| True Sequence: | TAG CAA G*G TCC GCT ACT GGC |
| Called Sequence: | TAG CAA GTG TCC *CT ACT GGC |

As will be appreciated in view of the disclosures herein, coincidental FNRs and FLDs cause insertion and deletion errors that cannot be corrected algorithmically and will remain undiscovered if the true sequence is not known. In other words, a base is called incorrectly when more than half of the single-molecule sensors 105 in the aligned sequence give the wrong answer. The probability of such events depends on the rate at which chemistry failures occur (the value of r). As explained above, the examples presented herein use high error rates in order to illustrate the application of the error-correction techniques. The error rates in a practical implementation should be significantly lower, thereby reducing the likelihood of the error-correction procedure not being able to correct errors. The disclosed error-correction techniques can be used to properly align multiple sensor 105 outputs at the inquiry steps. This can be accomplished using deep understanding of the physical origins of the possible error types (e.g., knowledge that certain sequences are impossible for the source nucleic acid), their average rates of occurrence, and their signatures in the sensor sequence output. Error-correction algorithms can be computationally intensive and difficult to implement if the chemistry error rates are high and the signatures of errors are obscured. The discussion below describes how the probability of an incorrect base-call depends on the read-length, cluster size N (for CLUS devices), number of sensors K sensing instances of the same nucleic acid strand (for SMAS devices 100), and failed chemistry error rates.

General Quantitative Result for Cluster Sequencer

A simple quantitative model is developed here for estimating the probability of an incorrect base-call in a cluster sequencer employing the modified additive sequencing protocol introduced above. The various types of errors (FNIs, FLRs, FNRs, and FLDs) are assumed to occur randomly throughout the cluster at rate r, where 0<r<1. Initially the cluster strands are in-phase with each other (e.g., synchronized, aligned, not out of synch), and the detected signal is proportional to the cluster size (N). The signal is detected when the complementary labeled nucleotides are introduced and successfully incorporated. No signal should be detected when non-complementary nucleotides are introduced during the inquiry cycle having A?⇒T?⇒C?⇒G? inquiry steps. Errors occur at rate r, which causes a gradually-increasing number of strands to be out of phase (not in synch) with the ensemble average. This reduces the intensity (or amplitude) of the ensemble signal when complementary nucleotides are incorporated and increases the intensity or amplitude of the background signal when non-complementary nucleotides are introduced. The average signal intensity at an inquiry step where labels should be detected because matching nucleotides are introduced and successfully incorporated (ON-State) is given by:

$$〈 1 〉 = \frac{N}{2} ( 1 + {e}^{- r \times C} ) , \qquad ( Eq . 1 ( a ) )$$

where C is the detection inquiry step (or number). Similarly, the intensity at an inquiry step where labels should not be detected because non-complementary nucleotides are introduced (OFF-State) is given by:

$$〈 0 〉 = \frac{N}{2} ( 1 - {e}^{- r \times C} ) \qquad ( Eq . 1 ( b ) )$$

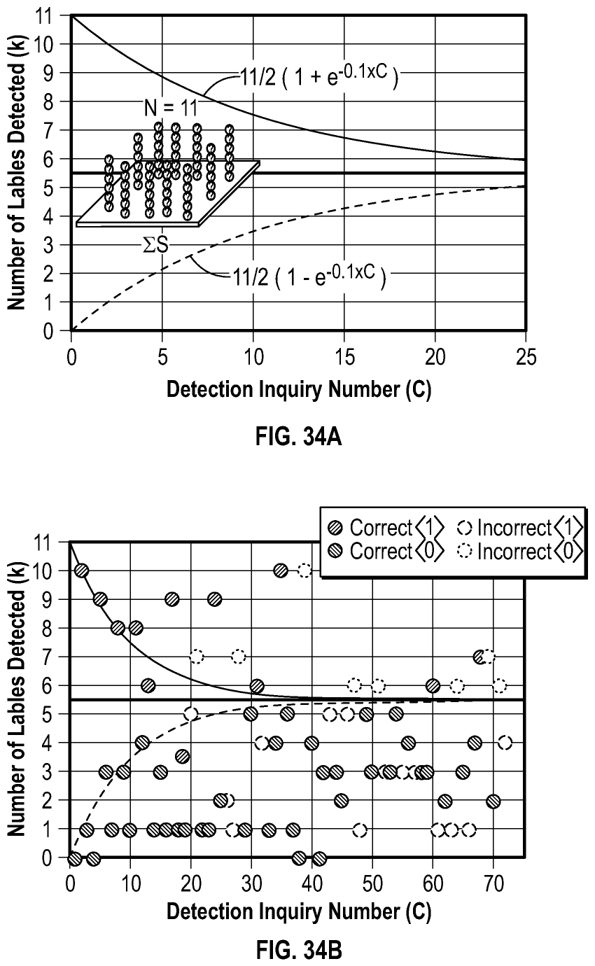

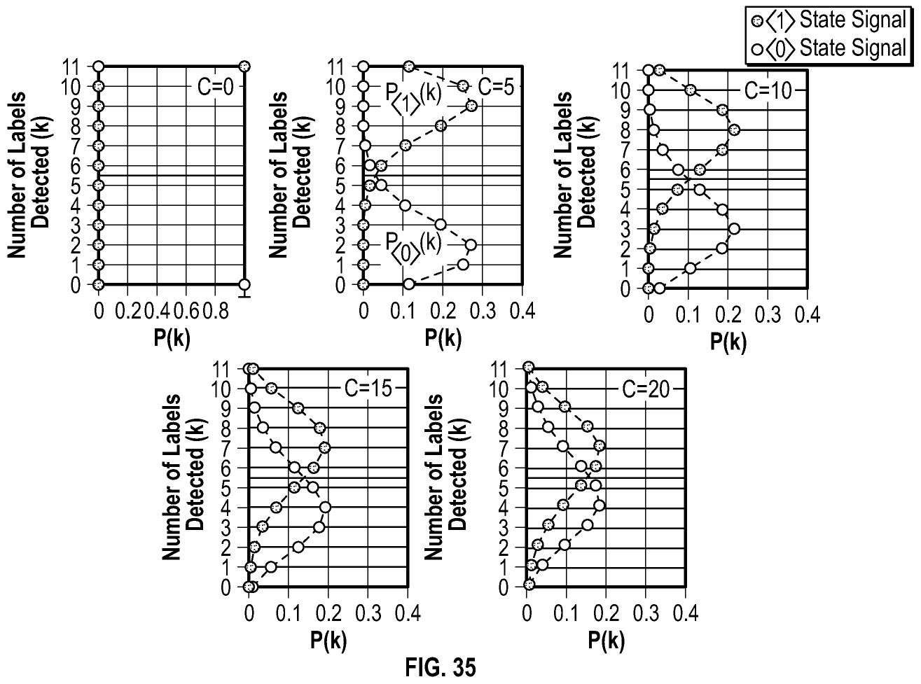

This background signal is generated by out-of-phase nucleic acid strands that incorporate nucleotides that are non-complementary to the in-phase position of the ensemble average. The functions from Eq. 1(a) and (b) are plotted in FIG. 34A for N=11 and r=0.1. FIG. 34B illustrates how the functions fit to the measured intensities from the cluster model example described previously. As illustrated, bases are called correctly until C≅15, but frequent errors occur at larger values of C.

As illustrated by FIGS. 34A and 34B, during the early sequencing inquiries (C small), the [symbol] 1 [symbol] and [symbol] 0 [symbol] states are well separated, but they quickly approach the average value N/2 following the functional forms represented by Eq. 1(a) and (b). Also, because error occurrences are random independent events, the measured signal of the two states is discretely distributed around their ensemble average values [symbol] 1 [symbol] and [symbol] 0 [symbol] . Specifically, the probability that measured ON-State intensity of a cluster size N is k when the ensemble average is [symbol] 1 [symbol] is given by Poisson distribution:

$${P}_{〈 1 〉} ( k ) ≅ {e}^{- 〈 1 〉} \times \frac{{〈 1 〉}^{N - k}}{( N - k ) !} \qquad ( Eq . 2 ( a ) )$$

Similarly, the probability that the recorded OFF-State intensity of the same cluster is k when the ensemble average is [symbol] 0 [symbol] is:

$${P}_{〈 0 〉} ( k ) ≅ {e}^{- 〈 0 〉} \times \frac{{〈 0 〉}^{k}}{k !} \qquad ( Eq . 2 ( b ) )$$

The probability functions [symbol] (k) and [symbol] (k) for N=11, r=0.1 and C=0, 5, 10, 15 and 20 are plotted in FIG. 35. The figure reveals two Poisson distributions with increasingly overlapping tails as C increases. The sum over all possible values of [symbol] (k) under the two discrete distributions is equal to 1:

$${\sum }_{k = 0}^{k = N} {P}_{〈 0 or 1 〉} ( k ) ≅ {\sum }_{k = 0}^{k = ∞} {P}_{〈 0 or 1 〉} ( k ) = 1 \qquad ( Eq . 3 )$$

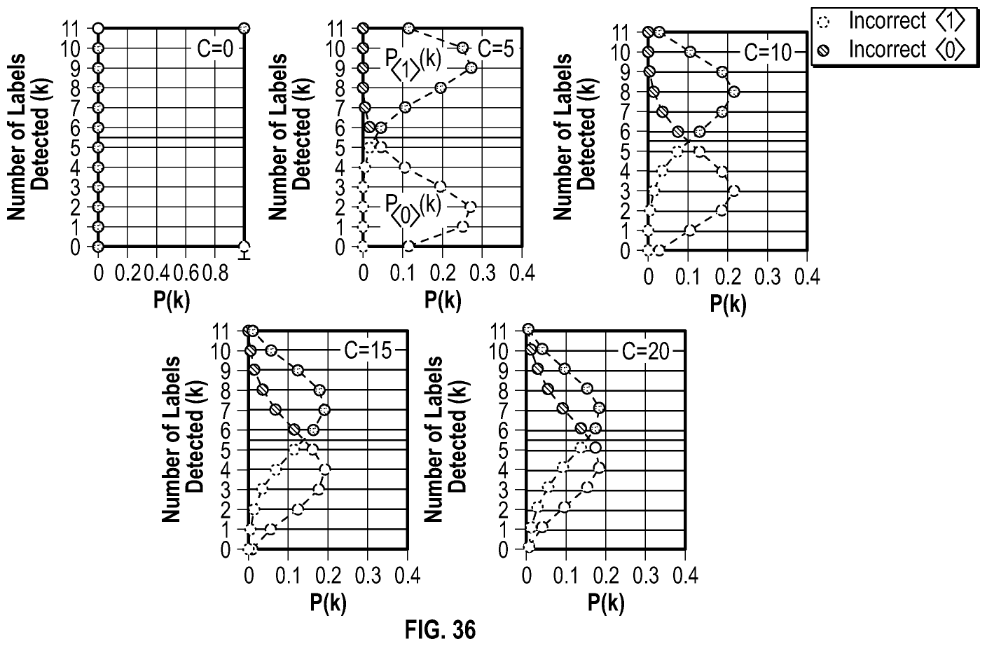

A base-call error is made when an ON-State is mistaken for an OFF-State or vice versa. FIG. 36 illustrates the discrete probability functions of the ON-State [symbol] (k) and the OFF-State [symbol] (k) for N=11 and r=0.1 at different sequencing inquiry steps C=0, 5, 10, 15 and 20. The sources of incorrect base-calls are shown as patterned dots when the tail of [symbol] (k) extends above the N/2 mid-value or as dashed circles when [symbol] (k) extends below the N/2 mid-value. The probability of making a wrong base-call becomes significant when the tail of the ON-State distribution extends considerably below k=N/2 (Incorrect [symbol] 1 [symbol] in FIG. 36), or the tail of the OFF-State distribution (Incorrect [symbol] 0 [symbol] ), extends above k N/2.

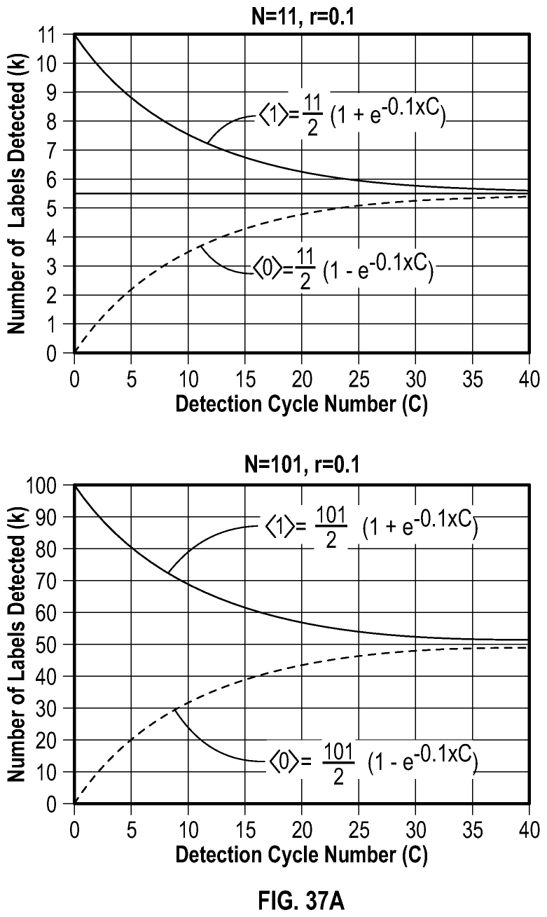

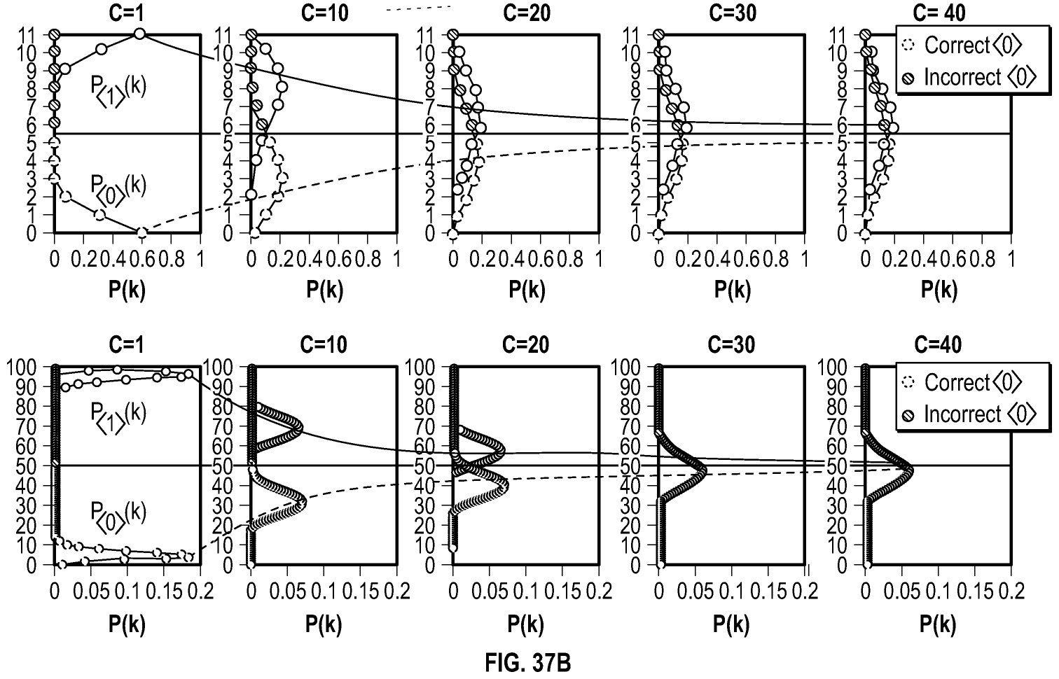

FIG. 37A shows the average ON-State and OFF-State intensity plots as a function of C for r=0.1 and cluster sizes of N=11 (top) and N=101 (bottom). FIG. 37B illustrates the OFF-State probability distribution function [symbol] (k) at C=1, 10, 20, 30 and 40 for r=0.1 and cluster sizes of N=11 (top) and N=101 (bottom). Increasing the cluster size delays the onset of base-calling errors by reducing the relative width of the [symbol] (k) distribution, which increases the distance from [symbol] .

In general, the probability of an incorrect base-call at sequencing inquiry number C, for cluster size N and chemistry failure rate r, denoted as P_{C,N,r}, is the sum of the probabilities that the OFF-State is called incorrectly, i.e., it is the sum over [symbol] (k) values for k values above k=(N+1)/2. These are the patterned dots in FIGS. 36 and 37B. Increasing the cluster size N increases the initial separation between the two discrete distribution peaks and delays the onset of base-calling errors. To simplify further discussion, only cases where the cluster size N is odd are considered to avoid uncertainty introduced when the detected signal is N/2, which is neither an ON-State nor an OFF-State. For odd values of N, P_{C,N,r} is given by:

$${P}_{C , N , r} ≅ {\sum }_{k = \frac{N + 1}{2}}^{k = N} {P}_{〈 0 〉} ( k ) = {\sum }_{k = \frac{N + 1}{2}}^{k = N} \{ {e}^{\frac{N}{2} ( {e}^{- r \times C} - 1 )} \times \frac{{[ \frac{N}{2} ( {e}^{- r \times C} - 1 ) ]}^{k}}{k !} \} \qquad ( Eq . 4 ( a ) )$$

Alternatively, P_{C,N,r} is the sum of probabilities that the ON-State is called incorrectly, i.e., it is the sum over [symbol] (k) values for values of k below k=(N−1)/2 (circles with backslash filling in FIG. 36), which is given by:

$${P}_{C , N , r} ≅ {\sum }_{k = 0}^{k = \frac{N - 1}{2}} {P}_{〈 1 〉} ( k ) = {\sum }_{k = 0}^{k = \frac{N - 1}{2}} \{ {e}^{\frac{N}{2} ( {e}^{- r \times C} + 1 )} \times \frac{{[ \frac{N}{2} ( {e}^{- r \times C} + 1 ) ]}^{N - k}}{( N - k ) !} \} \qquad ( Eq . 4 ( b ) )$$

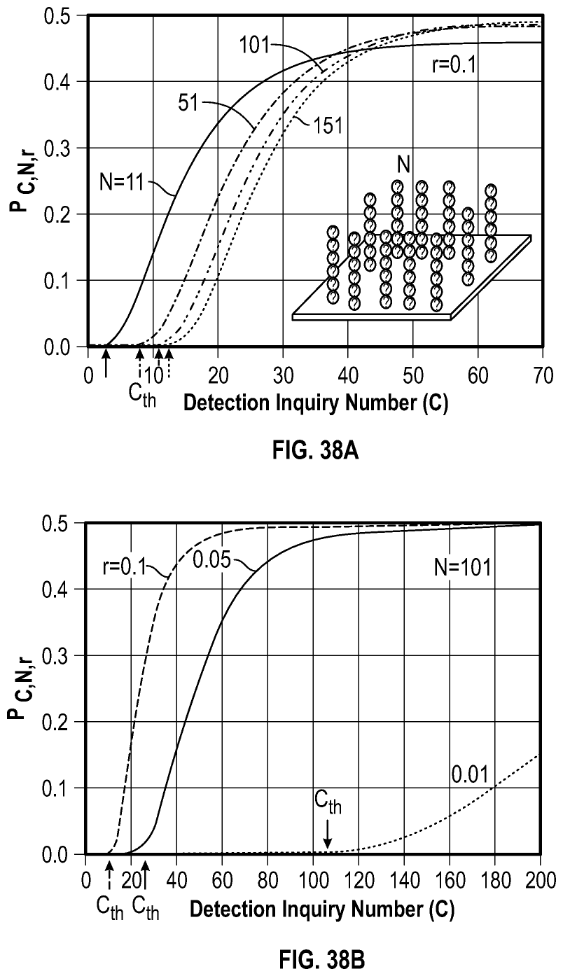

FIGS. 38A and 38B plot Eq. 4(a) and 4(b) as a function of C for various combinations of N and r. FIG. 38A plots the calculated P_{C,N,r} (C) functions for r=0.1 and N=11, 51, 101, and 151, and FIG. 38B plots the calculated P_{C,N,r} (C) functions for N=101 and r=0.1, 0.05, and 0.01. The plots show a dramatic rate of increase in the probability of incorrect base-calls at various threshold values Cₜₕ. As FIGS. 38A and 38B indicate, P_{C,N,r} approaches 0.5 as C goes to infinity. The plots in FIGS. 38A and 38B reveal the behavior characteristic of sequencers that analyze molecular ensembles (e.g., CLUS devices). The probability of an incorrect base-call (P_{C,N,r}) remains low when C is small, but it increases dramatically at a particular threshold (Cₜₕ), which is determined by the magnitudes of the N and r parameters. P_{C,N,r} approaches 0.5 as C goes to infinity, at which point the intensity of an ON-State is equal to that of an OFF-State, and there is 1 in 2 chance of making an incorrect base-call. P_{C,N,r} depends strongly on the three parameters C, N, r. Dependence on C is particularly important, as Cₜₕ imposes a limit on how many consecutive bases can be called before the probability of making an error becomes too large.

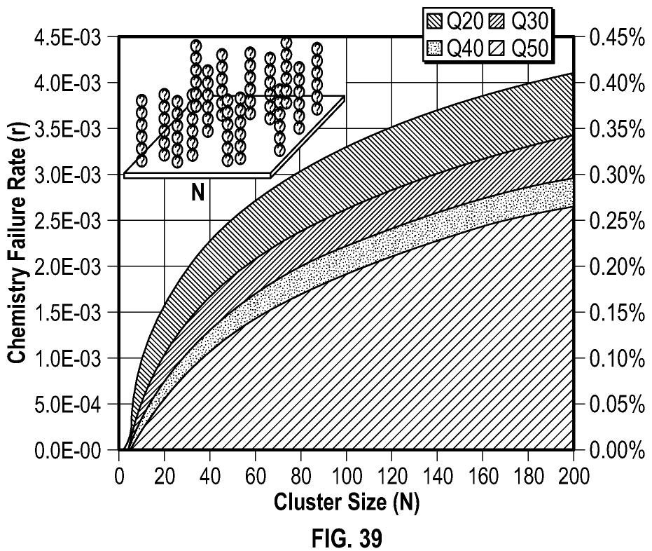

FIG. 39 illustrates the N-r parameter space where the probabilities of an incorrect base-call at position 150 (P_{C=375,N,r}) are lower than 1 in 100 (Q20), 1 in 1,000 (Q30), 1 in 10,000 (Q40) and 1 in 100,000 (Q50). Increasing the cluster size N, or reducing the chemistry failure rate r, pushes the threshold Cₜₕ to higher C values, but, as shown quantitatively in FIG. 39, the cluster sizes are rather large and the allowed chemistry error rates must be small to make a DNA sequencer suitable for diagnostic applications.

Currently, the benchmark in the sequencing industry is the ability to read 150 consecutive bases with 1 in 1,000 probability of making an incorrect base-call at position 150. This is generally referred to as Q30, but considerably larger sequencing quality factors of Q40 and even Q50 with longer read lengths are desired to detect rare mutations in high-precision diagnostics. The general expressions for P_{C,N,r} in Eq. 3(a) and (b) fully explore the C-N-r parameter space and can be used to estimate error tolerances and cluster size requirements for any sequencing metric. FIG. 39 shows the regions of the N-r parameter space where the probabilities of an incorrect base-call at position 150 (C≅375) are lower than 1 in 100 (Q20), 1 in 1,000 (Q30), 1 in 10,000 (Q40), and 1 in 100,000 (Q50). For example, if the average cluster size N in a sequencing array is 100 molecules, and the required sequencing precision is Q30 with 150 bp long reads (C=375), the allowed chemistry failure rate is r≤0.002641, i.e., only 26 or fewer out of individual single-molecule reactions across the sequencer array are allowed to fail at any sequencing inquiry step. If the required precision is Q50, only 19 or fewer errors per 10,000 reactions are permitted. If the average cluster size N is reduced to 10 molecules, the number drops to approximately 6 (Q30) and approximately 1 per 10,000 reactions (Q50).

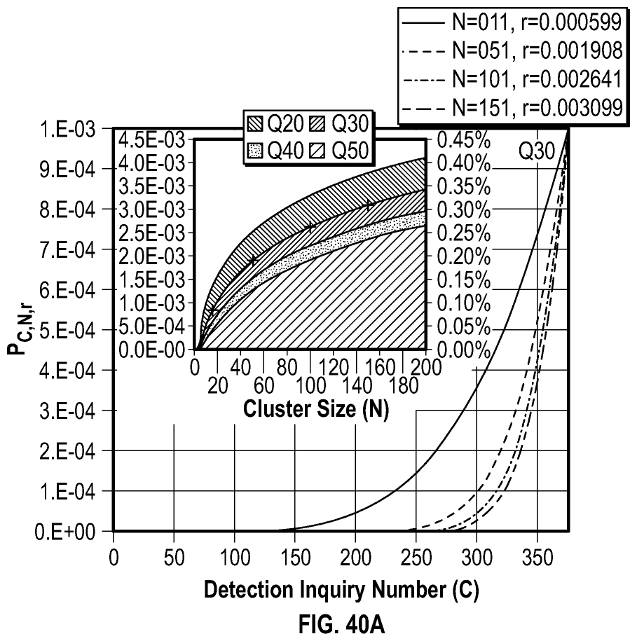

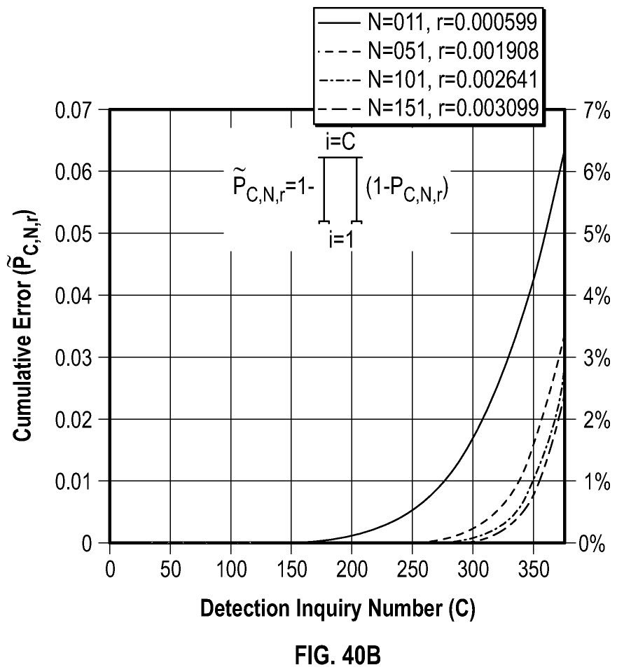

FIG. 40A shows the calculated P_{C,N,r} (C) along the Q30 contour for various N-r combinations, marked in the in the inset with crosses (“+” signs), all intersecting at P_{C,N,r} (C=375)=0.001. The plots reveal that increasing the cluster size N not only boosts the tolerance for chemistry failures, but it also delays the onset of base-calling errors by pushing by pushing the threshold Cₜₕ to higher C values, which leads to lower cumulative errors. If the probability of making an incorrect base-call at inquiry cycle C is P_{C,N,r}, the probability of making a correct call is (1−P_{C,N,r}). The probability of making C consecutive correct calls is then:

$$( 1 - {P}_{1 , N , r} ) \times ( 1 - {P}_{2 , N , r} ) \times ( 1 - {P}_{3 , N , r} ) \times … \times ( 1 - {P}_{C , N , r} ) ≡ {\prod }_{j = 1}^{j = C} ( 1 - {P}_{j , N , r} ) \qquad ( Eq . 5 ( a ) )$$

The probability of not making C correct base-calls in a row, which is the same the probability of making at least one error at any inquiry cycle C or smaller (or the cumulative error probability \tilde{P}_{C,N,r}) is given by:

$${\tilde{P}}_{C , N , r} = 1 - {\prod }_{j = 1}^{j = C} ( 1 - {P}_{j , N , r} ) \qquad ( Eq . 5 ( b ) )$$

where P_{j,N,r} is given by Eq. 4(a) or (b). FIG. 40B plots the calculated cumulative error probabilities, \tilde{P}_{C,N,r}(C), along the same contours and illustrates that larger clusters generate lower cumulative errors. Finally, it is instructive to calculate and plot the N-r parameter space marking the regions where the cumulative probability of an incorrect base-call at position 150 (the target read length in some embodiments) is less than or equal to 1 in 100 (\tilde{Q}20), 1 in 1,000 (\tilde{Q}30), 1 in 10,000 (\tilde{Q}40) and 1 in 100,000 (\tilde{Q}50). FIG. 41 illustrates the N-r parameter space where the cumulative probabilities of an incorrect base-call at position 150 (\tilde{P}_{C=375,N,r}) are less than or equal to 1 in 100 (\tilde{Q}20), 1 in 1,000 (\tilde{Q}30), 1 in 10,000 (\tilde{Q}40), and 1 in 100,000 (\tilde{Q}50). The plot in FIG. 41 shows quantitatively that a CLUS sequencer may include large DNA cluster sizes N to benefit from ensemble behavior, and it may require very reliable chemistry (only a few dozens of failures per 10,000 reactions are allowed) for high-precision diagnostic applications. More specifically, if the average cluster in a sequencing array holds on average, e.g., 100 molecules, and the particular sequencing application tolerates a probability of cumulative base-calling errors of 1 in 1,000 (\tilde{Q}30), only approximately 22 or fewer out of 10,000 individual single-molecule reactions across the sequencer array are allowed to fail at any sequencing inquiry step. The plot in FIG. 41 illustrates that an increase in sequencing throughput by reducing the cluster size N and packing more clusters into the sensing area can only be achieved with parallel improvements in sequencing chemistry. The rate of needed improvement accelerates as the cluster size N becomes smaller, and the CLUS device can no longer benefit from the large ensemble behavior.

General Quantitative Result for Single-Molecule Array Sequencer

To compare CLUS and SMAS platforms, a simple quantitative model is developed to estimate the probability of incorrect base-call in a SMAS device 100. Unlike the ensemble case applicable to CLUS devices (described above), in which little to no error correction can be implemented, the ability of SMAS devices 100 to individually sequence and record detection results corresponding to individual nucleic acid molecules allows the development and implementation of powerful techniques to identify and eliminate at least some of the errors in the resulting data record(s). One or more error-correction techniques, as disclosed herein, may be applied to data generated from a sequencing procedure (e.g., SBS) before base-calls are made to identify and correct errors in the detection results to improve the accuracy of the called sequence. Specifically, the alignment of detection results from multiple sensors 105 at some or all of the inquiry steps of the sequencing procedure can be improved. Incorrect base-calls can still be made even when the error-correction algorithm is successful in aligning multiple sensor detection results correctly. As explained above, coincidental FNR errors and FLD errors can cause insertion and deletion errors that might not be corrected. Depending on the number of errors in the data records (which is determined in part by chemistry failure rates), the error correction process can be complex and computationally intensive, but it will be appreciated that modern processors have sufficient computational power to carry out even the most computationally intensive of the disclosed techniques.

Below, a general case of K single-molecule sensors 105 of a SMAS device 100, each capable of monitoring a single instance of clonal DNA, is considered. As in the analysis of the CLUS device above, it is assumed that the four types of errors (FNIs, FLRs, FNRs and FLDs) occur randomly during the sequencing procedure and are distributed throughout the inquiry steps.

As explained above, in some embodiments, a probabilistic error-correction algorithm is implemented (e.g., by at least one processor 130, which may be included in the SMAS device 100 or external to the SMAS device 100). In some embodiments, the probabilistic error-correction algorithm improves the alignment of at least some sensor 105 detection results in a data record. In some embodiments, some or all of the error-correction algorithm is implemented after some or all inquiry steps have been completed and some or all data has been captured. As described previously, the error-correction procedure essentially eliminates FNIs and FLRs, as well as some FLDs. The algorithmic re-alignment of sensor 105 detection results also makes the probability of making an incorrect base-call independent of the inquiry step number C. Also, because the error-correction algorithm re-aligns at least some sensor 105 detection results in the data record(s), thereby correcting at least some of the errors, the effective error rate r is smaller than in the CLUS case. Following application of the exemplary error-correction algorithm, in some embodiments, bases are called incorrectly only when more than half of the K sensors 105 in the algorithmically aligned sequence give an incorrect result.

The probability of making an incorrect base-call (P_{K,r}) is only a function of (a) the number, K, of sensors 105 sequencing instances of the same nucleic acid molecule (which may be fewer than all of the sensors 105 in the sensor array 110), and (b) the chemistry failure rate r. Similarly to the approach taken for the analysis of the CLUS device above, the value of K is restricted to odd values to avoid the case in which exactly half of the sensors 105 disagree with the other half. The probability of making an incorrect base-call is given by:

$${P}_{K , r} = ( \begin{matrix}K \\ ( K + 1 ) / 2\end{matrix} ) {{r}^{( K + 1 ) / 2} ( 1 - r )}^{( K - 1 ) / 2} ++ ( \begin{matrix}K \\ ( K + 3 ) / 2\end{matrix} ) {{r}^{( K + 3 ) / 2} ( 1 - r )}^{( K - 3 ) / 2} ++ ( \begin{matrix}K \\ ( K + 5 ) / 2\end{matrix} ) {{r}^{( K + 5 ) / 2} ( 1 - r )}^{( K - 5 ) / 2} ++ ( \begin{matrix}K \\ ( K + 7 ) / 2\end{matrix} ) {{r}^{( K + 7 ) / 2} ( 1 - r )}^{( K - 7 ) / 2} + … + ( \begin{matrix}K \\ K\end{matrix} ) {r}^{K} , \qquad ( Eq . 6 )$$

$$where ( \begin{matrix}a \\ b\end{matrix} ) = \frac{a !}{b ! ( a - b ) !} . For example , if K = 3 , \\ {P}_{K = 3 , r} = ( \begin{matrix}3 \\ 2\end{matrix} ) {r}^{2} ( 1 - r ) + ( \begin{matrix}3 \\ 3\end{matrix} ) {r}^{3} = 3 {r}^{2} ( 1 - r ) + {r}^{3} = 3 {r}^{2} - 2 {r}^{3} \qquad ( Eq . 7 )$$

In the example of K=3, the multiplicative

$$( \begin{matrix}3 \\ 2\end{matrix} ) = 3$$

term accounts for cases in which 2 out of 3 sensors 105 suffer from errors (e.g., they incorrectly detect a label (FLR, FNR) or incorrectly fail to detect a label (FNI, FLD)) at a particular inquiry step simultaneously, thereby forcing an incorrect base-call. Denoting the three sensors 105 as S1, S2, and S3, this situation occurs when: (1) S1 and S2 suffer from errors simultaneously, (2) S1 and S3 suffer from errors simultaneously, or (3) S2 and S3 suffer from errors simultaneously. The

$$( \begin{matrix}3 \\ 3\end{matrix} ) = 1$$

term accounts for the improbable case that all three sensors S1, S2, and S3 simultaneously suffer from errors, which also results in an incorrect base call. Because the largest term in the polynomial expansion is r^{K-1} and 0<r<1, the probability of making an incorrect base-call drops dramatically by increasing the number of single-molecule sensors 105 (i.e., increasing the value of K).

For example, if r=0.1, P_{K=3,r=0.1}=0.029, which means there is approximately a 3 in 100 chance of making an incorrect base-call. Stated another way, approximately 4.35 out of 150 base-calls will be incorrect on average, which is too large for some diagnostic applications. In order to use three nanoscale sensors 105 to sequence with Q30 (P_{K,r}=0.001), the chemistry failure rate would need to be reduced to r=0.01837, meaning that only approximately 19 out of 1,000 inquiries would be permitted to be in error. If the number of sensors 105 (the value of K) is increased to 11, however, failure of over 12 out of a hundred reactions would be tolerated.

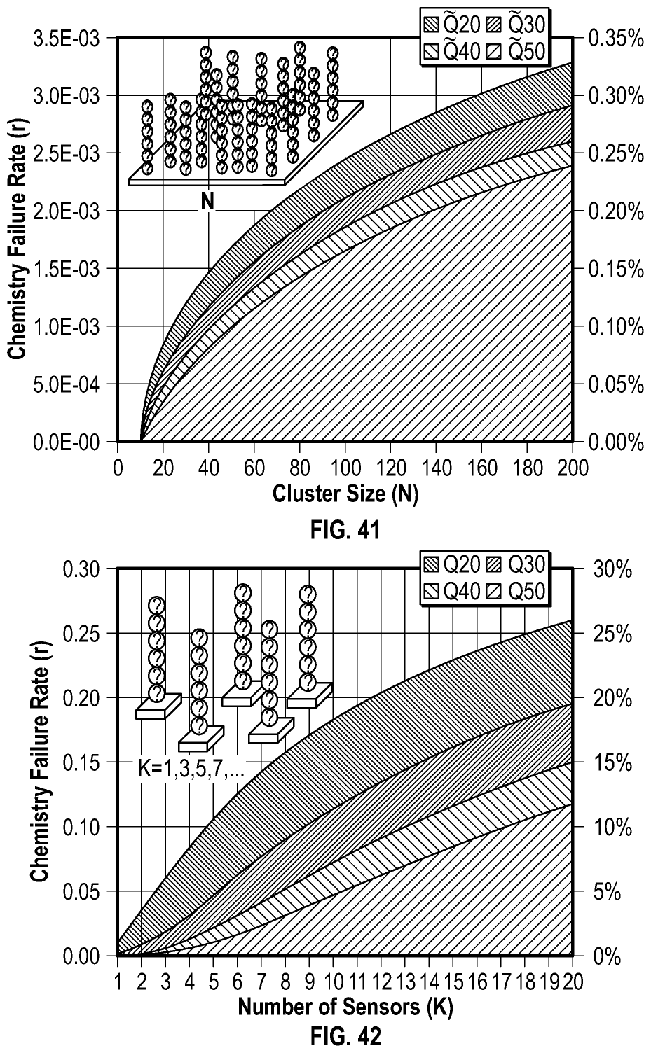

As done above for CLUS devices, the K-r parameter space is explored below for SMAS devices 100 to identify the regions where the probabilities of an incorrect base-call at any inquiry position are lower than 1 in 100 (Q20), 1 in 1,000 (Q30), 1 in 10,000 (Q40), and 1 in 100,000 (Q50). FIG. 42 illustrates the calculated results for the K-r parameter space where the probability of an incorrect base-call at every inquiry step (P_{K,r}) is lower than 1 in 100 (Q20), 1 in 1,000 (Q30), 1 in 10,000 (Q40) and 1 in 100,000 (Q50). As shown in FIG. 42, if the number K of single-molecules sensors 105 sensing instances of the same nucleic acid molecule is 11, and the required sequencing precision is Q30, the allowed chemistry failure rate is r≲0.13, meaning that as many as about 13 out of 100 individual single-molecule reactions among those 11 sensors 105 are allowed to fail. If the required precision is Q50, about 6 or fewer errors per 100 reactions are permitted among the 11 sensors 105.

As a comparison with FIG. 39 indicates, the allowed error rates for the SMAS device 100 are considerably larger that the rates allowed for the CLUS device, although that result alone does not equitably compare the two platforms because the probability of making an incorrect base-call in a CLUS device (P_{C,N,r}) is very low during early inquiry steps and increases suddenly at a threshold inquiry step, Cₜₕ. This phenomenon was discussed in relation to FIG. 39. On the other hand, for a SMAS device 100, the probability of an incorrect base-call (P_{K,r}) stays constant throughout the inquiry steps and therefore results in larger cumulative errors.

A more equitable way to compare the performances of CLUS devices and SMAS devices 100 is to compare cumulative error probabilities for the two device types. Eq. 5(b) above represents the cumulative error probability for a CLUS device. The cumulative error probability for SMAS devices 100 can also be derived. The probability of making an incorrect base-call at every inquiry step C is P_{K,r} (Eq. 6), and therefore the probability of making a correct call is (1−P_{K,r}). The probability of making C correct calls in a row is then (1−P_{K,r})^{C}, and the cumulative error probability (\tilde{P}_{K,r}) is

\tilde{P}_{K,r}=1−(1−P_{K,r})^{C} (Eq. 8)

FIGS. 43A and 43B show the cumulative probabilities of an incorrect base-call at position 150 for CLUS devices and SMAS devices 100. Eq. 5(b) can be used, for example, to calculate the probability of a CLUS device making an incorrect base-call at any base position smaller than or equal to 150. FIG. 43A shows the K-r parameter space for the CLUS device and marks the regions where the cumulative probability of an incorrect base-call at position 150 is less than or equal to 1 in 100 (\tilde{Q}20), 1 in 1,000 (\tilde{Q}30), 1 in 10,000 (\tilde{Q}40), and 1 in 100,000 (\tilde{Q}50) for a CLUS device. FIG. 43B evaluates Eq (8) and shows the K-r parameter space marking the regions where the cumulative probability of an incorrect base-call at position 150 is less than or equal to 1 in 100 (\tilde{Q}20), 1 in 1,000 (\tilde{Q}30), 1 in 10,000 (\tilde{Q}40), and 1 in 100,000 (\tilde{Q}50) for a SMAS device 100.

A comparison of FIGS. 43A and 43B reveals that SMAS devices 100 are potentially superior sequencing platforms to CLUS devices. SMAS devices 100 can have a smaller footprint (as explained in the discussions of, e.g., FIGS. 7A, 7B, 9A, 9B, and 10) and can be considerably more error tolerant than CLUS devices. Use of SMAS devices 100 promises higher throughput, lower error rates, and longer read lengths compared to CLUS devices, which are larger and rely on large molecular ensembles. Development of a commercially viable SMAS device 100 and/or system may use some or all of (a) high-precision nanoscale fabrication of densely-packed sensors 105 capable of recognizing individual labels, (b) optimization of chemistry steps to reduce error rates to acceptable levels, and/or (c) availability of effective bioinformatics tools to adjust the alignment, in a data record, of sequencing data from at least some nanoscale sensors 105 by probabilistically eliminating errors.

### Exemplary SMAS Sequencing Procedure

As explained above, improvements to the sequencing throughput of a CLUS device can be achieved by reducing the cluster size N (thereby packing more clusters into the device) if the rate of sequencing chemistry failures is also reduced, which may be challenging. In contrast, a feasible realization of an error-tolerant, ultra-high-throughput SMAS device 100 using large arrays of single-molecule binding sites 116 in accordance with some embodiments is presented below. For purposes of example, it is assumed that the SMAS device 100 sequences DNA, but it is to be appreciated that, in general, any kind of nucleic acid may be sequenced.

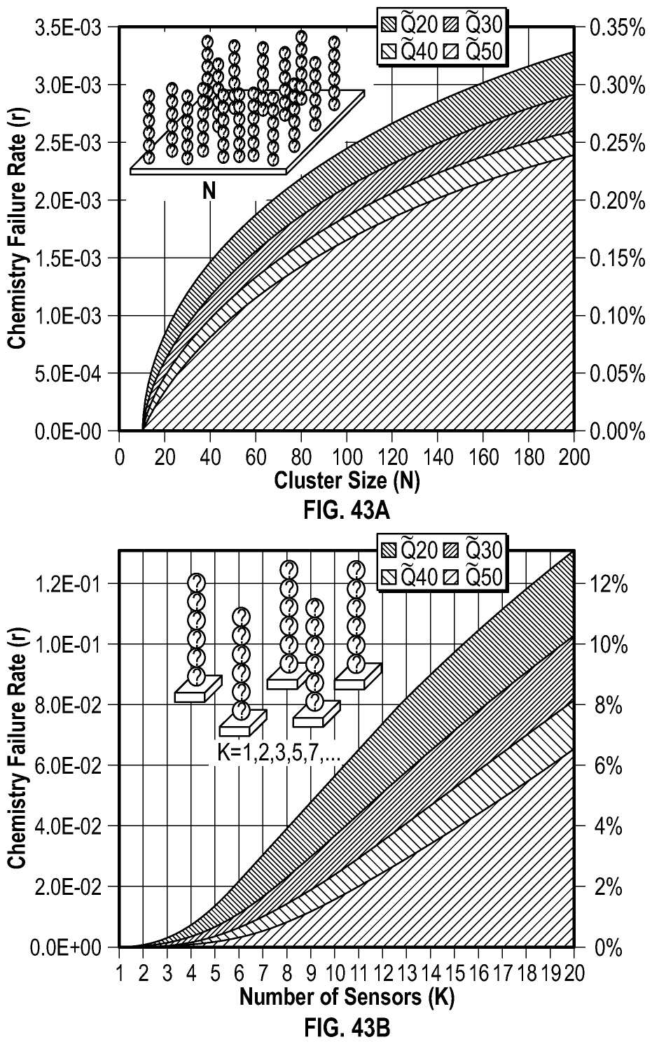

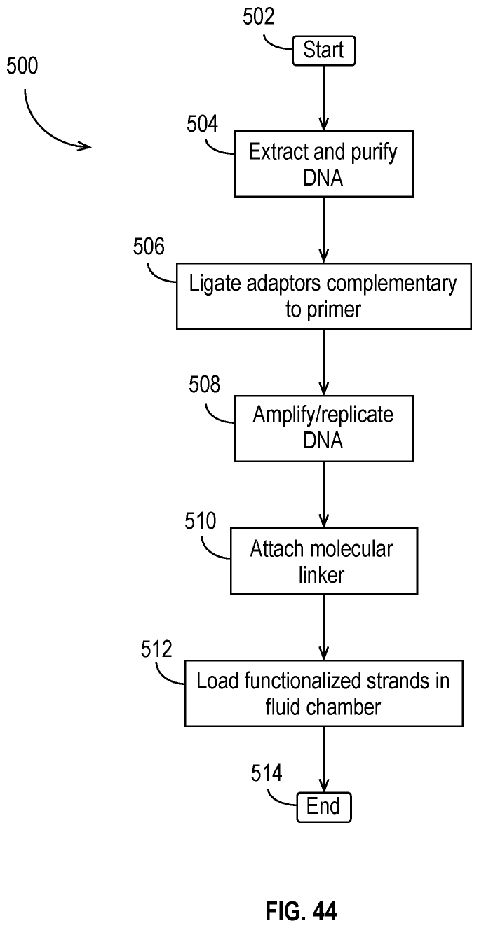

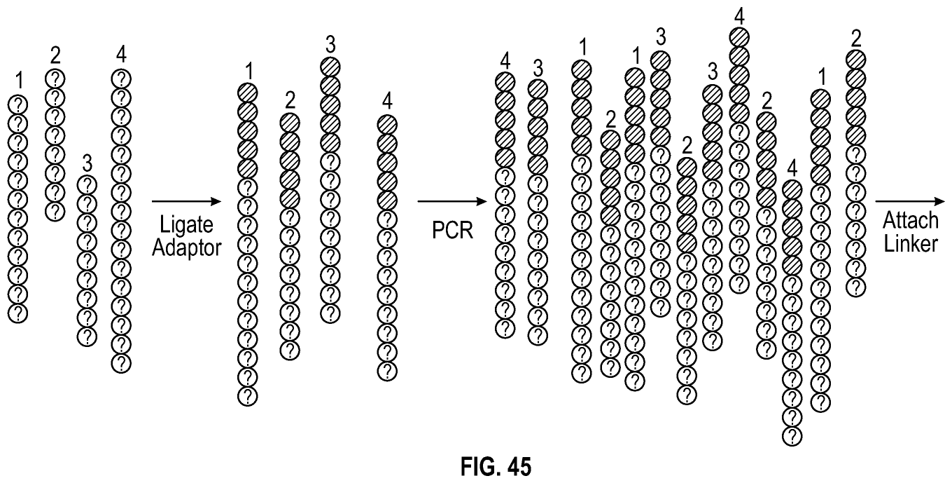

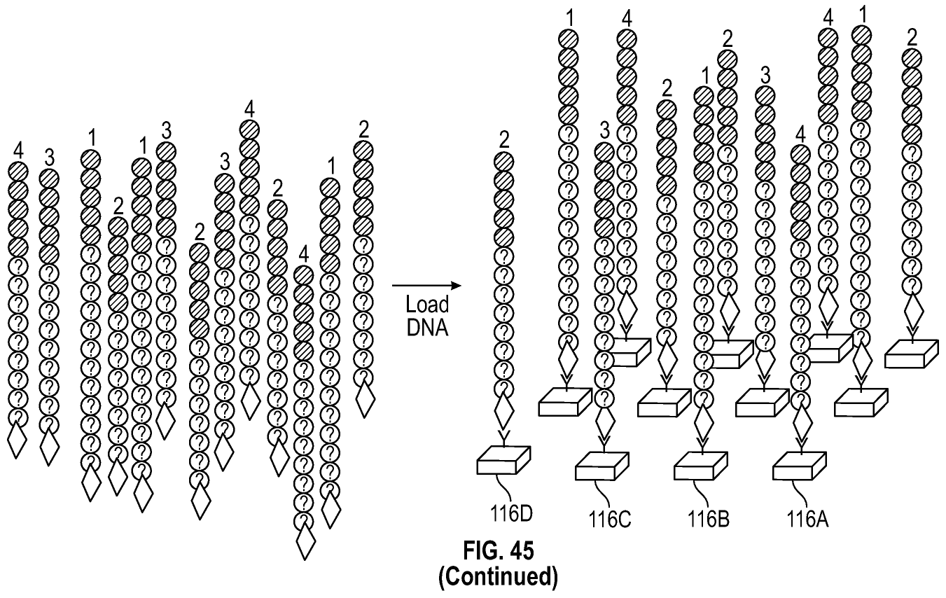

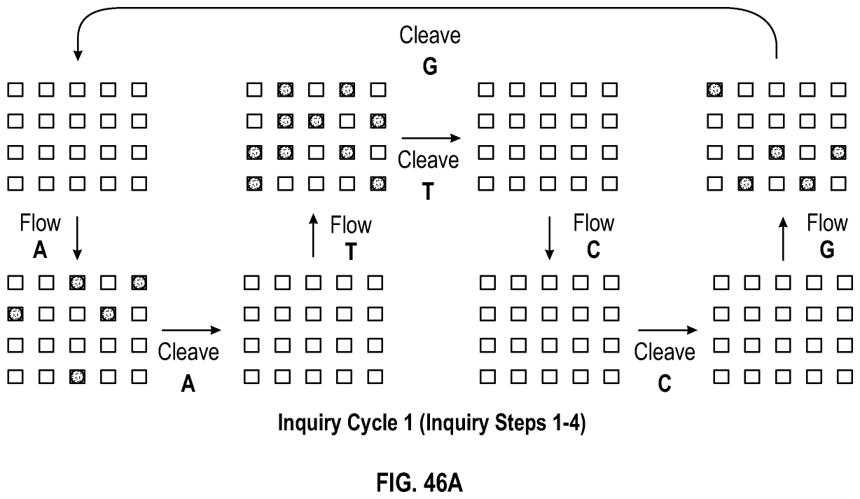

FIGS. 44 and 45 illustrate an exemplary sample preparation and loading process 500 in accordance with some embodiments. FIG. 44 is a flow diagram illustrating the process 500, and FIG. 45 illustrates the results of various steps of the process 500. In some embodiments, the sample preparation and loading process 500 begins at 502. At 504, DNA extraction and purification is performed, which results in several extracted DNA fragments 505 as shown in FIG. 45. At 506, an adaptor complementary to the primer is ligated to one end (e.g., 3′) of the extracted DNA to produce the strands 507 shown in FIG. 45. At 508, PCR (or some other replication technique) is performed to generate multiple (ideally, identical) instances of the extracted strands, shown as 509 in FIG. 45. At 510, a molecular linker capable of creating a strong bond (e.g., by click chemistry) to the chemically functionalized surface of the fluid chamber 115 (the binding sites 116) of the SMAS device 100 is attached to the other end (e.g., 5′) of the ssDNA fragments, thereby producing the strands 511 shown in FIG. 45. At 512, the functionalized strands 511 are loaded into the fluid chamber 115 and scattered randomly among and bound to the binding sites 116. As shown in the right-most portion of FIG. 45, each of the binding sites 116 supports no more than a single DNA strand. (Although each binding site 116 can support no more than one strand, it is to be understood that there is no requirement that every binding site 116 must support a DNA strand. Fewer than all of the binding sites 116 of the SMAS device 100 can be used, whether on purpose or by chance.) Assuming the extracted DNA fragments 503 are different from each other, as a result of the sample preparation and loading process 500, there will be multiple instances of each of the extracted DNA fragments 505 within the fluid chamber 115, but their positions are unknown. At 514, the exemplary sample preparation and loading process 500 ends.

A benefit of the exemplary sample preparation and loading process 500 is that it simplifies DNA amplification, which can be performed in bulk, off-device, using (for example) conventional PCR, before the DNA strands are added to the SMAS device 100. In contrast, when a CLUS device is used, amplification (e.g., bridge amplification) is executed only after the DNA fragments have been added to the CLUS device in order to create arrays of contiguous clusters of amplified DNA.

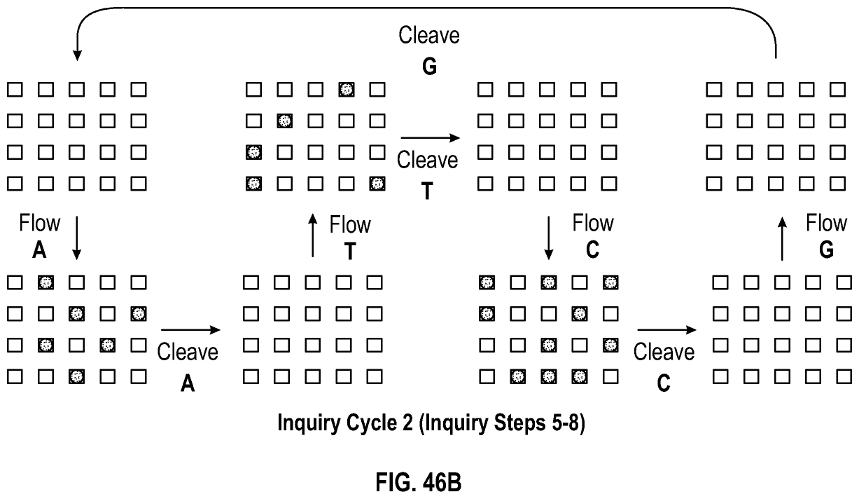

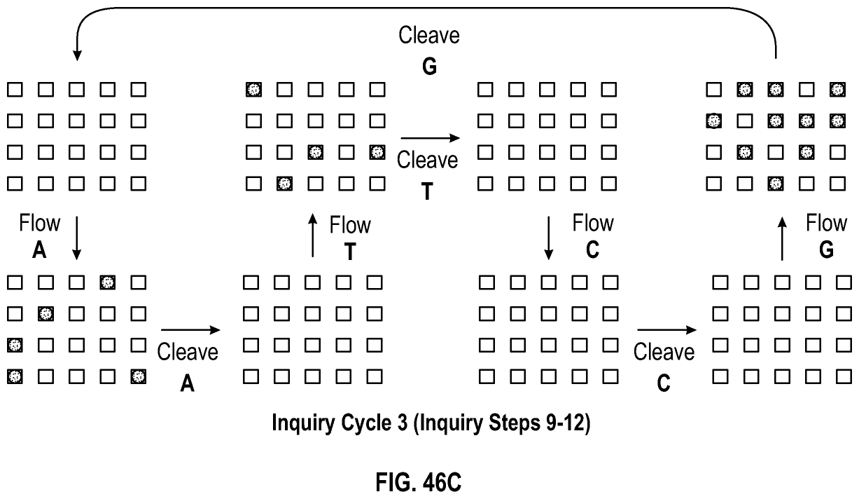

After the sample preparation and loading process 500 has been performed, base-calling may be performed using, for example, the additive approach, the subtractive approach, or the modified additive approach introduced above. FIGS. 46A, 46B, and 46C illustrate simulated detection results (the sensors 105 detect labels) using the modified additive approach during three exemplary inquiry cycles (A?⇒T?⇒C?⇒G? each, for a total of 12 inquiry steps) performed by an example SMAS device 100 with a sensor array 110 that has 20 sensors 105 (and 20 binding sites 116) arranged in four rows and five columns. Multiple instances of four different DNA strands are randomly distributed throughout the sensor array 110, but their particular positions within the sensor array 110 and their sequences are initially not known.

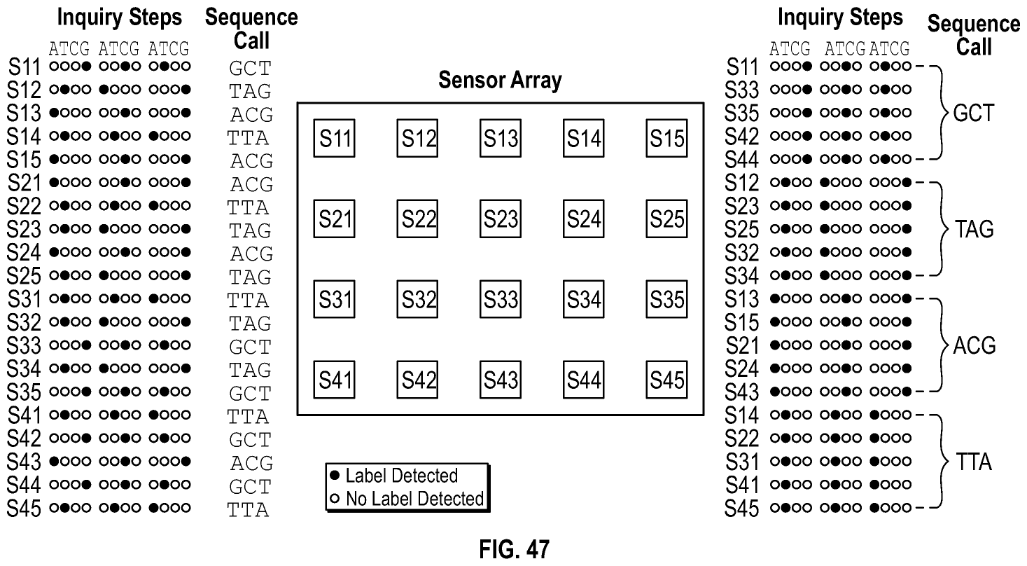

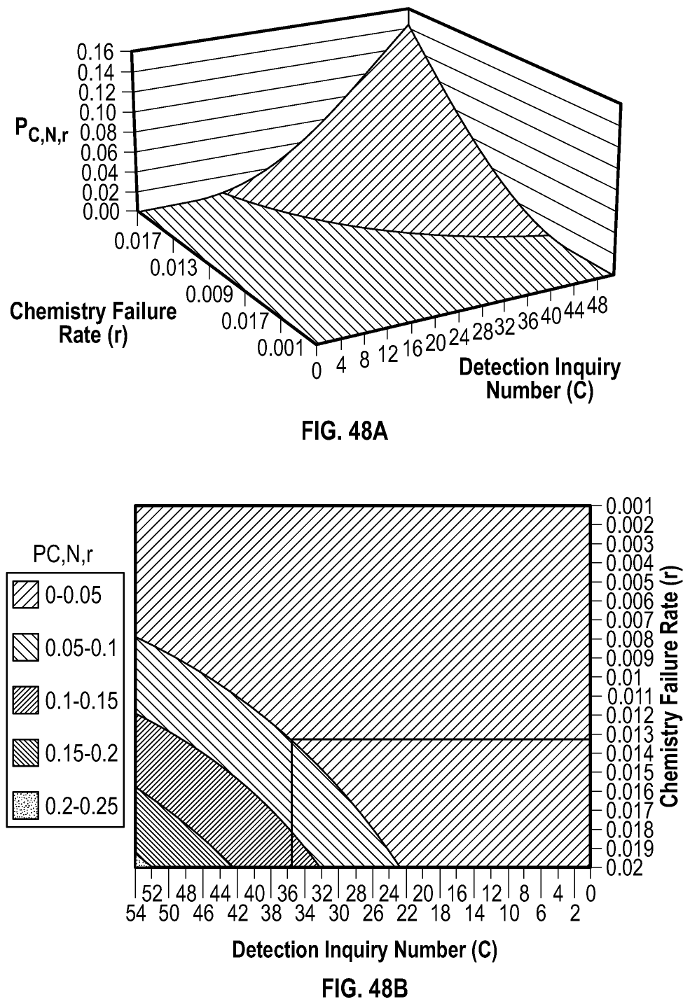

FIG. 47 illustrates how the detection data illustrated in FIGS. 46A, 46B, and 46C can be rearranged to call the bases and reveal the positions of the different DNA strands. FIG. 47 provides a table showing the output of every sensor 105 in the exemplary array at individual inquiry steps, and the resulting base-calls resulting in the called sequence. The right-hand portion of FIG. 47 reorders the sensors 105 to group the detection results of sensors 105 that are sensing instances of the same DNA strand. As shown in FIG. 47, four sequences are called: GCT (Strand #1), TAG (Strand #2), ACG (Strand #3), and TTA (Strand #4).

If errors (FNIs, FLRs, FNRs, or FLDs) occur during the inquiry steps, some of the detection results (label detected or label not detected) will be incorrect, and the deterministic and/or probabilistic error detection and/or correction techniques described above can be implemented to detect and eliminate at least some errors, as long as the identities of those sensors 105 that sense instances of the same DNA strand are determined. Recall that instances of a particular DNA strand may be attached to binding sites 116 scattered throughout the fluid chamber 115, and their positions are not generally known when the sequencing process begins. Once the process is initiated, during each inquiry step, each of a plurality of S sensors 105 detects labels at its respective binding site 116. To perform the error correction, subgroups of the S sensors 105 that are sequencing instances of the same nucleic acid strand are identified.

Consider a very large sensor array 110 (e.g., 4 billion binding sites 116 and 4 billion respective sensors 105) with 400 million different DNA strands, each approximately 150 bases long. This means that there are approximately 10 instances of each unique DNA strand distributed randomly throughout the fluid chamber 115 (and the binding sites 116 and the sensor array 110). It is also assumed for the sake of example that the sequences are random. Assuming a reasonably low error rate r, after the first inquiry cycle, almost all of the binding sites 116 (and sensors 105) holding (sensing) DNA instances starting with A will have been identified, as will those holding (sensing) T, and those holding (sensing) C, and those holding (sensing) G. About 10⁹ sensors 105 will detect labels indicating the first base is A, about 10⁹ sensors 105 will detect labels indicating the first base is T, about 10⁹ sensors will detect labels indicating the first base is C, and about 10⁹ sensors will detect labels indicating the first base is G. After the second inquiry cycle, almost all of the binding sites 116 (and sensors 105) holding (sensing) DNA instances starting with all 16 possible combinations (AA, AT, AC, AG, TA, TT, TC, TG, CA, CT, CC, CG, GA, GT, GC, and GG) will have been identified. About 2.5×10⁸ sensors will detect labels indicating the first and second bases are AA, about 2.5×10⁸ sensors will detect labels indicating the first and second bases are AT, about 2.5×10⁸ sensors will detect labels indicating the first and second bases are AC, etc. In general, after some number D of label detections (or C≅2.5×D inquiry steps assuming the modified additive approach is used for sequencing), all 4^{D}=4^{2C/5} binding sites 116 holding DNA strands that start with some sequence that is D-bases long will be identified. This means that the average size of a group of sensors 105 sensing instances of the same DNA strand in a SMAS device 100 with a 4 billion-sensor array 110 is 4×10⁹/(4^{2C/5}).

Because our example has approximately 10 instances of every unique strand on average, it will take approximately C≅35 inquiry cycles to identify the positions of binding sites 116 that hold instances of a particular strand. Assuming use of the modified additive approach, about 14 bases will have been identified during the process. Considerably fewer inquiry steps will be likely needed in reality for diagnostic applications because the human genome is not random, and not all the mathematically possible sequences are represented. The identities (locations) of the binding sites 116 holding instances of the same DNA strand can be determined in even fewer steps if a specific set of genes is targeted during DNA extraction, which further reduces the number of possible sequences of bases and facilitates binding site 116 identification.

The confidence that the correct set of binding sites 116 has been identified increases with the number of inquiry steps, but so does the probability of making an detection error (e.g., incorrectly detecting a label or incorrectly failing to detect a label). Multiple errors can occur during initial inquiry cycles while the binding sites 116 holding instances of the same strands are being identified. The results derived for the CLUS device suggest that this may not be an issue. For example, FIG. 38A shows that the CLUS device's probability of making an incorrect base call is very small during the early inquiry steps, and it is only when the threshold Cₜₕ is reached that the probability of error increases sharply. Also recall that the base-calling accuracy of a SMAS device 100 is the same as for a CLUS device if no error correction is applied because the SMAS device 100 would simply report the ensemble result by summing up individual sensor 105 results.

Consider, for example, the 4 billion-sensor-array example above and consider one set of 11 sensors 105 (K=11) monitoring instances of a particular DNA strand distributed randomly throughout the binding sites 116. Now treat them as an ensemble (K=N=11), as if the binding sites 116 were forming a cluster and only the combined characteristics (e.g., signals) of their respective sensors 105 were measured. FIGS. 48A and 48B plot the calculated probability of making an incorrect base-call, P_{C,N,r}, given by Eqs. 4(a) and (b) as a function of the inquiry step number C and chemistry failure rate r. The curve in FIG. 48A marks the approximate position of the threshold in C-r space where P_{C,N,r} suddenly increases. FIG. 48B is a top view of the contour plot shown in FIG. 48A and clearly indicates the chemistry failure tolerance for a 4-billion-sensor SMAS device 100 containing, on average, approximately 10 instances of each DNA strand. The positions (identities) of the approximately 10 binding sites 116 (and sensors 105) holding (sensing) instances of each unique DNA strand can be determined reliably as long as the error probability remains low through approximately 35 inquiry steps. This puts the limit on the maximum allowed chemistry failure rate at 0.013, i.e., 13 out of 1,000 detection events would be tolerated. The calculated results in FIG. 48A and FIG. 48B indicate that if the chemistry failure rate stays below approximately 13 incorrect detection events per 1,000, the 4-billion-sensor SMAS device 100 should be able to establish the positions within the fluid chamber 115 (and among binding sites 116 and among sensors 105) of all of the instances of all billion different DNA strands. Once those positions are established, the error-correction techniques described herein can be implemented to eliminate errors that occur during the remaining approximately 340 inquiry steps (assuming use of the modified additive approach).

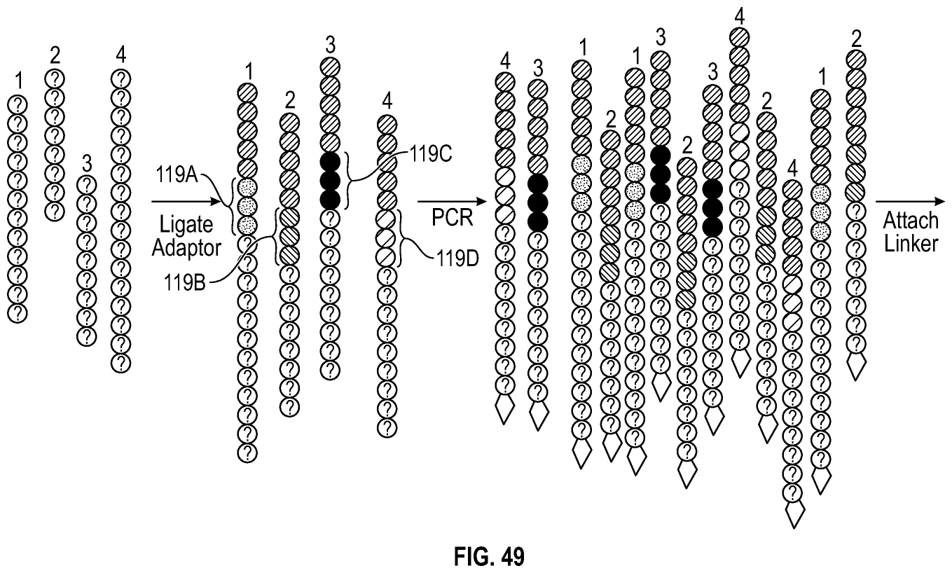

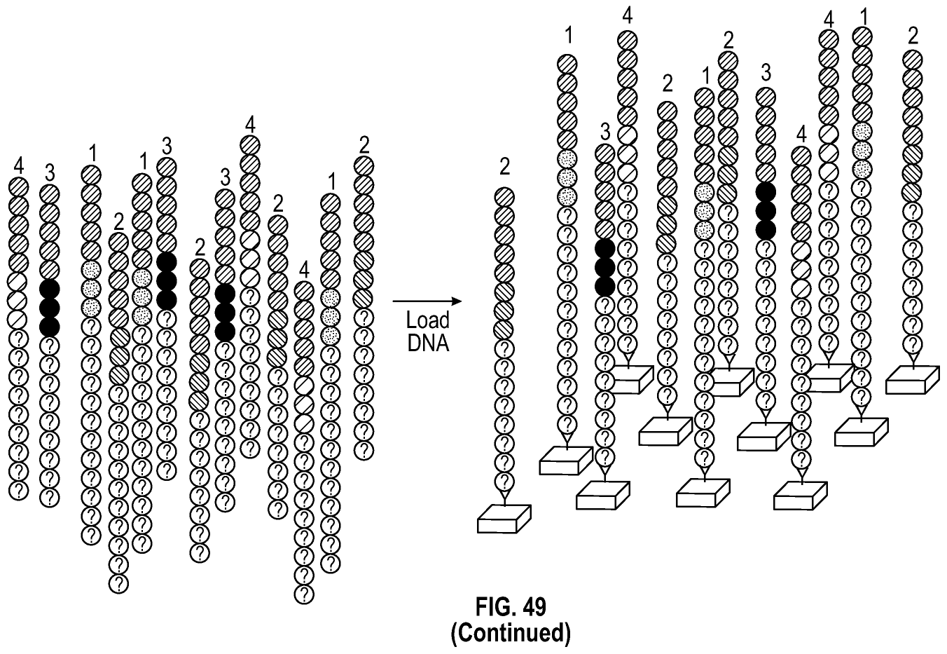

If the chemistry error rate is expected or known to be too high, such that errors are likely to plague the first approximately 35 inquiry steps, alternative approaches can be used to help identify the binding sites 116 that carry instances of the same DNA strand. For example, different unique barcodes can be ligated to the primer end in subsets of extracted DNA so that a known sequence is read during the early sequencing cycles. FIG. 49 illustrates the use of barcodes in sample preparation and DNA loading in accordance with some embodiments. As shown in FIG. 49, unique barcodes are ligated to the extracted DNA to facilitate recognition of sites holding instances of the same DNA in presence of sequencing errors. For example, FIG. 49 shows four unique DNA strands, each of which is assigned a unique barcode (e.g., strand 1 is assigned the barcode 119A, strand 2 is assigned the barcode 119B, strand 3 is assigned the barcode 119C, and strand 4 is assigned the barcode 119D). If the barcodes are significantly different from each other, they should be easily identifiable even if the chemistry failure rate is very high. As will be appreciated, the appropriate number of unique barcodes could be high for high throughput diagnostic applications.

The exemplary 4-billion-sensor SMAS device 100 described herein is considered a fairly-high-throughput sequencer by the current standards. Such a SMAS device 100 provides approximately 150 Giga-base (Gb) reads during a single run, which rivals the output of state-of-the-art high-end sequencing systems introduced in 2020.

It is to be appreciated that there are many ways to implement the devices, systems, and methods disclosed herein. For example, a system for nucleic acid sequencing may consist of a single device (e.g., a SMAS device 100 that includes all of the hardware and software to perform the disclosed operations), or it may include a SMAS device 100 and other components that together perform the disclosed operations. For example, a system may comprise a SMAS device 100 that performs a nucleic acid sequencing procedure and saves detection results from that sequencing procedure, and at least one processor external to the SMAS device 100 (e.g., in an external computer) that performs error detection and correction on the saved detection results and calls the bases.

FIG. 50 illustrates an exemplary system 160 in accordance with some embodiments. The system 160 comprises (i.e., includes but is not limited to) a fluid chamber 115, a plurality of S sensors 105, and at least one processor 130. Optionally, the system 160 includes memory 170 for storing records comprising detection results obtained during a sequencing procedure (e.g., one or more files having binary entries documenting whether, during each of a plurality of inquiry cycles, each of a plurality of S sensors 105 detected or did not detect at least one label). As shown by the dashed line in FIG. 50, if the system 160 includes memory 170, the at least one processor 130 may be communicatively coupled to the memory 170 so that the at least one processor 130 can store data in the memory 170 and/or retrieve data from the memory 170.

The fluid chamber 115 comprises a plurality of S binding sites, each of which is configured to bind no more than one strand of nucleic acid to be sequenced. FIG. 50 shows four binding sites 116, but it is to be appreciated that the system 160 can include more or fewer binding sites 116. Each of the S sensors 105 is configured to detect labels present in the fluid chamber 115. FIG. 50 shows four sensors 105, but it is to be appreciated that the system 160 can include more or fewer sensors 105. When the system 160 is in operation, each of the S sensors 105 detects labels attached to nucleotides incorporated into a respective strand of nucleic acid bound to a respective binding site 116 of the S binding sites 116. As explained previously, the sensors 105 may be magnetic sensors, optical sensors, or any other type of sensor that can detect the labels being used to label nucleotides. The fluid chamber 115, sensors 105, and binding sites 116 are described in detail above. Those descriptions apply to FIG. 50 and are not repeated here.

The at least one processor 130 is configured to execute one or more machine-executable instructions. The instructions, when executed, cause the at least one processor 130 to perform a sequencing procedure comprising a plurality of inquiry steps (e.g., as described in the context of any of FIGS. 11, 12, 14, 16, 44). Specifically, in operation, during inquiry steps of the sequencing procedure, the at least one processor 130 obtains a respective characteristic of each of the S sensors 105 (represented by the dashed lines between the at least one processor 130 and the sensors 105A, 105B, 105C, and 105D. The respective characteristic indicates whether the sensor 105 detects or does not detect a label (e.g., it indicates presence or absence of at least one label). The at least one processor 130 may interpret the obtained characteristic to determine whether the sensor 105 detects or does not detect the presence of a label. Based at least in part on the obtained respective characteristic, the at least one processor 130 records whether the respective sensor detected the presence or absence of at least one label during the inquiry step. The at least one processor 130 is also configured to perform an error-correction procedure on at least one record that contains results of the sequencing procedure. The error-correction procedure may operate on some or all of the records generated by the sequencing procedure, and it may operate on detection results from some or all of the inquiry steps of the sequencing procedure. For example, as described above, to apply the error-correction procedure, the at least one processor may identify and apply deterministic or probabilistic error-correction to a subset of K records, where each of the K records in the subset corresponds to detection results from a sensor 105 sensing an instance of the same nucleic acid strand. Sequencing procedures and error-correction procedures are described in detail above. Those descriptions apply to the system of FIG. 50, and the at least one processor 130, and are not repeated here.

The at least one processor 130 may be implemented by a general or special purpose processor (or set of processing cores) and thus may execute sequences of programmed instructions to effectuate the various operations associated with obtaining sensor 105 characteristics, performing error-correction procedures, and/or interaction with a user, system operator, or other system components.

The at least one processor 130 of the system 160 may be a single processor (e.g., in a SMAS device 100), or it may comprise multiple processors, which may be co-located (e.g., in a SMAS device 100) or physically separated from each other. For example, a first portion of the at least one processor 130 may be included in a SMAS device 100, and a second portion of the at least one processor 130 may be external to the SMAS device 100. In embodiments in which the at least one processor 130 comprises first and second portions, the first portion may be responsible for obtaining the characteristics of the sensors 105, determining on the basis of the characteristics whether the sensors 105 detected labels during an inquiry cycle, and recording (e.g., in memory 170) whether each of the S sensors 105 detected the presence or absence of at least one label during the inquiry cycle, and the second portion may be responsible for obtaining a record of detection results and performing an error-correction procedure. Alternatively, the first portion may be responsible for obtaining the characteristics of the sensors 105, determining on the basis of the characteristics whether each of the sensors 105 detected at least one label during an inquiry cycle, and providing indications of whether the sensors 105 detected labels to another entity over a communication interface (e.g., a wireless or wired interface, such as Ethernet, Wi-Fi, etc.). In such implementations, the second portion of the at least one processor 130 may be responsible for obtaining a record of the detection results (e.g., a file having binary entries documenting whether, during each inquiry cycle, each of a plurality of S sensors 105 detected or did not detect at least one label) provided by the first portion of the at least one processor 130, performing an error-correction procedure, and calling bases. In the foregoing description and in the accompanying drawings, specific terminology has been set forth to provide a thorough understanding of the disclosed embodiments. In some instances, the terminology or drawings may imply specific details that are not required to practice the invention.

To avoid obscuring the present disclosure unnecessarily, well-known components are shown in block diagram form and/or are not discussed in detail or, in some cases, at all.

The section headings provided in the detailed description are solely for convenience or reference and are not intended to be limiting. The section headings in no way define, limit, construe, or describe the scope or extent of such sections. Also, although various specific embodiments have been disclosed, it will be evident that various modifications and changes may be made thereto without departing from the broader spirit and scope of the disclosure. For example, features or aspects of any of the embodiments may be applied in combination with any other of the embodiments or in place of counterpart features or aspects thereof.

Certain of the techniques and methods disclosed herein (e.g., obtaining detection results from sensors 105, performing error-correction procedures, etc.) and/or user interfaces for configuring and managing them may be implemented by machine execution of one or more sequences instructions (including related data necessary for proper instruction execution). Such instructions may be recorded on one or more computer-readable media for later retrieval and execution within one or more processors of a special purpose or general purpose computer system or consumer electronic device or appliance. Computer-readable media in which such instructions and data may be embodied include, but are not limited to, non-volatile storage media in various forms (e.g., optical, magnetic, or semiconductor storage media) and carrier waves that may be used to transfer such instructions and data through wireless, optical, or wired signaling media or any combination thereof. Examples of transfers of such instructions and data by carrier waves include, but are not limited to, transfers (uploads, downloads, e-mail, etc.) over the Internet and/or other computer networks via one or more data transfer protocols (e.g., HTTP, FTP, SMTP, etc.).

Unless otherwise specifically defined herein, all terms are to be given their broadest possible interpretation, including meanings implied from the specification and drawings and meanings understood by those skilled in the art and/or as defined in dictionaries, treatises, etc. As set forth explicitly herein, some terms may not comport with their ordinary or customary meanings.

As used in the specification and the appended claims, the singular forms “a,” “an” and “the” do not exclude plural referents unless otherwise specified. The word “or” is to be interpreted as inclusive unless otherwise specified. Thus, the phrase “A or B” is to be interpreted as meaning all of the following: “both A and B,” “A but not B,” and “B but not A.” Any use of “and/or” herein does not mean that the word “or” alone connotes exclusivity.

As used in the specification and the appended claims, phrases of the form “at least one of A, B, and C,” “at least one of A, B, or C,” “one or more of A, B, or C,” and “one or more of A, B, and C” are interchangeable, and each encompasses all of the following meanings: “A only,” “B only,” “C only,” “A and B but not C,” “A and C but not B,” “B and C but not A,” and “all of A, B, and C.”

To the extent that the terms “include(s),” “having,” “has,” “with,” and variants thereof are used in the detailed description or the claims, such terms are intended to be inclusive in a manner similar to the term “comprising,” i.e., meaning “including but not limited to.”

The terms “exemplary” and “embodiment” are used to express examples, not preferences or requirements.

The term “coupled” is used herein to express a direct connection/attachment as well as a connection/attachment through one or more intervening elements or structures.

The terms “over,” “under,” “between,” and “on” are used herein refer to a relative position of one feature with respect to other features. For example, one feature disposed “over” or “under” another feature may be directly in contact with the other feature or may have intervening material. Moreover, one feature disposed “between” two features may be directly in contact with the two features or may have one or more intervening features or materials. In contrast, a first feature “on” a second feature is in contact with that second feature.

The term “substantially” is used to describe a structure, configuration, dimension, etc. that is largely or nearly as stated, but, due to manufacturing tolerances and the like, may in practice result in a situation in which the structure, configuration, dimension, etc. is not always or necessarily precisely as stated. For example, describing two lengths as “substantially equal” means that the two lengths are the same for all practical purposes, but they may not (and need not) be precisely equal at sufficiently small scales. As another example, a structure that is “substantially vertical” would be considered to be vertical for all practical purposes, even if it is not precisely at 90 degrees relative to horizontal.

The drawings are not necessarily to scale, and the dimensions, shapes, and sizes of the features may differ substantially from how they are depicted in the drawings.

Although specific embodiments have been disclosed, it will be evident that various modifications and changes may be made thereto without departing from the broader spirit and scope of the disclosure. For example, features or aspects of any of the embodiments may be applied, at least where practicable, in combination with any other of the embodiments or in place of counterpart features or aspects thereof. Accordingly, the specification and drawings are to be regarded in an illustrative rather than a restrictive sense.

Without prejudice, and without surrender of any subject matter, please amend the claims as follows:

1. A system comprising:
a plurality of S binding sites, each of the S binding sites configured to bind no more than one strand of nucleic acid to be sequenced;
a plurality of S sensors configured to detect labels, each of the S sensors for sensing a respective strand of nucleic acid bound to a respective binding site of the S binding sites; and
at least one processor configured to execute one or more machine-executable instructions that, when executed, cause the at least one processor to:
(a) at each inquiry step of a plurality of M inquiry steps of a sequencing procedure, and for each of the S sensors:
obtain a respective characteristic of the respective sensor, wherein the respective characteristic indicates presence or absence of at least one label, and
based at least in part on the obtained respective characteristic, record whether the respective sensor detected the presence or absence of at least one label during the inquiry step, and
(b) perform an error-correction procedure on at least one record, the at least one record comprising results of the sequencing procedure for at least a subset of the S sensors at each of the M inquiry steps, wherein perform the error-correction procedure on the at least one record comprises:
identify, based on at least a portion of the at least one record, a plurality of candidate sequences associated with instances of a particular nucleic acid strand, and
determine or estimate which of the plurality of candidate sequences is most likely to be correct.

2-3. (canceled)

4. The system recited in claim 1, wherein each of the plurality of S sensors is configured to detect at least one of fluorophores, magnetic particles, charged molecules, or organometallic complexes.

5-25. (canceled)

26. The system recited in claim 1, wherein determine or estimate which of the plurality of candidate sequences has a highest probability of being correct comprises:
determine, for each of the plurality of candidate sequences, a respective metric; and
based at least in part on the respective metrics and a criterion, choosing a particular candidate sequence as most likely to be correct.

27. The system recited in claim 26, wherein the respective metrics are likelihoods of occurrence, and wherein the criterion is a minimum likelihood of occurrence or a threshold likelihood of occurrence.

28. (canceled)

29. The system recited in claim 1, wherein determine or estimate which of the plurality of candidate sequences has a highest probability of being correct comprises eliminate at least one of the plurality of candidate sequences based on a known constraint on a nucleic acid sequence of the particular nucleic acid strand.

30. The system recited in claim 29, wherein the known constraint is an impossibility of a particular sequence of bases.

31. The system recited in claim 29, wherein determine or estimate which of the plurality of candidate sequences has the highest probability of being correct further comprises determine the known constraint based at least in part on a source of the particular nucleic acid strand.

32. The system recited in claim 1, wherein the at least one record comprises a collection of binary values, wherein a first binary value indicates that the label was detected, and a second binary value indicates that no label was detected, and wherein perform the error-correction procedure comprises:
identify, in the at least one record, a run of second binary values, and
delete the run of the second binary values from the at least one record.

33. (canceled)

34. The system recited in claim 1, wherein perform the error-correction procedure on the at least one record comprises:
identify, in the at least one record, a set of consecutive indications that no label was detected by a first sensor of the S sensors, and
delete the set of consecutive indications that no label was detected by the first sensor of the S sensors from the at least one record.

35. The system recited in claim 1, wherein perform the error-correction procedure on the at least one record comprises:
change at least one entry of the at least one record based on a majority result for a particular inquiry step.

36. A device for sequencing nucleic acid, the device comprising:
a fluid chamber comprising a plurality of S binding sites, each of the S binding sites configured to bind no more than one strand of nucleic acid to be sequenced;
a plurality of S magnetic sensors configured to detect labels present in the fluid chamber, each of the S magnetic sensors for sensing a respective strand of nucleic acid bound to a respective binding site of the S binding sites; and
at least one processor configured to execute one or more machine-executable instructions that, when executed, cause the at least one processor to, at each inquiry step of a plurality of M inquiry steps of a sequencing procedure, and for each of the S magnetic sensors:
obtain a respective characteristic of the respective magnetic sensor, wherein the respective characteristic indicates presence or absence of at least one label,
based at least in part on the obtained respective characteristic, determine whether the respective magnetic sensor detected the presence or absence of at least one label during the inquiry step, and
record, in a respective record associated with the respective magnetic sensor, whether the respective magnetic sensor detected the presence or absence of at least one label during the inquiry step.

37-38. (canceled)

39. The device recited in claim 36, wherein determining whether the respective magnetic sensor detected the presence or absence of the at least one label during the inquiry step comprises:
determining whether the obtained respective characteristic of the respective magnetic sensor meets or exceeds a threshold, or
comparing the obtained respective characteristic of the respective magnetic sensor to a previously-detected value.

40. (canceled)

41. The device recited in claim 39, wherein the previously-detected value is at least one of a baseline value, a frequency, a magnetic field, or a noise level.

42. (canceled)

43. The device recited in claim 36, wherein each of the plurality of S magnetic sensors is configured to detect at least one of magnetic particles, charged molecules, or organometallic complexes.

44-56. (canceled)

57. The device recited in claim 36, wherein, when executed by the at least one processor, the one or more machine-executable instructions further cause the at least one processor to:
perform an error-correction procedure on at least one record, the at least one record comprising results of the sequencing procedure for at least a subset of the S magnetic sensors at each of the M inquiry steps.

58. (canceled)

59. The device recited in claim 57, wherein perform the error-correction procedure on the at least one record comprises:
identify, based on at least a portion of the at least one record, a plurality of candidate sequences associated with instances of a particular nucleic acid strand, and
determine or estimate which of the plurality of candidate sequences is most likely to be correct.

60. The device recited in claim 59, wherein determine or estimate which of the plurality of candidate sequences is most likely to be correct comprises:
determine, for each of the plurality of candidate sequences, a respective metric; and
based at least in part on the respective metrics and a criterion, choose a particular candidate sequence as most likely to be correct.

61. The device recited in claim 60, wherein the respective metrics are likelihoods of occurrence, and wherein the criterion is a minimum likelihood of occurrence or a threshold likelihood of occurrence.

62. (canceled)

63. The device recited in claim 59, wherein determine or estimate which of the plurality of candidate sequences is most likely to be correct comprises eliminate at least one of the plurality of candidate sequences based on a known constraint on a nucleic acid sequence of the particular nucleic acid strand.

64. The device recited in claim 63, wherein the known constraint is an impossibility of a particular sequence of bases.

65. (canceled)

66. The device recited in claim 57, wherein the at least one record comprises a collection of binary values, wherein a first binary value indicates that the label was detected, and a second binary value indicates that no label was detected, and wherein perform the error-correction procedure comprises:
identify, in the at least one record, a run of second binary values, and
delete the run of the second binary values from the at least one record.

67. (canceled)

68. The device recited in claim 57, wherein perform the error-correction procedure on the at least one record comprises:
identify, in the at least one record, a set of consecutive indications that no label was detected, and
delete, from the at least one record, the set of consecutive indications that no label was detected.

69. The device recited in claim 57, wherein perform the error-correction procedure on the at least one record comprises:
change at least one entry of the at least one record based on a majority result for a particular inquiry step.

70. A method of sequencing a plurality of S nucleic acid strands using a sequencing device comprising a fluid chamber and a plurality of S sensors configured to detect labels present in the fluid chamber, each of the S sensors for sensing a respective nucleic acid strand bound to a respective one of a plurality of S binding sites within the fluid chamber, each of the S binding sites configured to bind no more than one strand of nucleic acid for sequencing, the method comprising:
binding the S nucleic acid strands to the S binding sites;
performing a sequencing procedure comprising M inquiry steps to produce S records, each of the S records capturing M detection results of a respective one of the S sensors, each of the M detection results indicating whether, during a respective one of the M inquiry steps, the respective one of the S sensors detected at least one label in the fluid chamber, wherein each of the M detection results in each of the S records is represented by a binary value; and
applying an error correction procedure to at least a subset of the S records to estimate a nucleic acid sequence of at least one of the S nucleic acid strands, wherein performing the sequencing procedure comprises:
in response to the respective one of the S sensors detecting the at least one label, recording a first binary value in a respective record of the S records, and
in response to the respective one of the S sensors not detecting the at least one label, recording a second binary value in the respective record of the S records.

71. The method recited in claim 70, wherein the subset of the S records captures results of the sequencing procedure for instances of a particular nucleic acid strand.

72. The method recited in claim 71, further comprising amplifying or replicating the particular nucleic acid strand to create the instances of the particular nucleic acid strand before binding the S nucleic acid strands to the S binding sites.

73. (canceled)

74. The method recited in claim 70, wherein each record of the at least a subset of the S records corresponds to a respective instance of a particular nucleic acid strand.

75. The method recited in claim 74, further comprising identifying the subset of the S records before applying the error correction procedure.

76. The method recited in claim 75, wherein identifying the subset of the S records is based on knowledge of a particular barcode associated with the particular nucleic acid strand.

77. The method recited in claim 75, wherein identifying the subset of the S records comprises identifying, in each record of the subset of the S records, a particular barcode associated with the particular nucleic acid strand.

78. The method recited in claim 75, wherein identifying the subset of the S records comprises identifying, in each record of the subset of the S records, a common sequence of entries.

79. The method recited in claim 70, wherein the sequencing procedure comprises:
(a) introducing a labeled nucleotide into the fluid chamber;
(b) rinsing away unbound molecules;
(c) obtaining a first characteristic from a first sensor of the plurality of S sensors;
(d) obtaining a second characteristic from a second sensor of the plurality of S sensors;
(e) determining, based on the first characteristic, whether the first sensor detected at least one label in the fluid chamber;
(f) determining, based on the second characteristic, whether the second sensor detected at least one label in the fluid chamber;
(g) recording a first indication in a first record of the S records, the first indication indicating whether the first sensor detected at least one label in the fluid chamber;
(h) recording a second indication in a second record of the S records, the second indication indicating whether the second sensor detected at least one label in the fluid chamber;
repeating (a) through (h) for at least one other labeled nucleotide; and
after repeating (a) through (h) for the at least one other labeled nucleotide, cleaving and rinsing away labels.

80. The method recited in claim 70, wherein the sequencing procedure comprises:
(a) introducing a plurality of labeled nucleotides into the fluid chamber, each of the plurality of labeled nucleotides using a respective linker;
(b) rinsing away unbound nucleotides;
(c) cleaving a first linker;
(d) obtaining a first characteristic from a first sensor;
(e) obtaining a second characteristic from a second sensor;
(f) determining, based on the first characteristic, whether the first sensor detected at least one label in the fluid chamber;
(g) determining, based on the second characteristic, whether the second sensor detected at least one label in the fluid chamber;
(h) recording a first indication in a first record of the S records, the first indication indicating whether the first sensor detected at least one label in the fluid chamber;
(i) recording a second indication in a second record of the S records, the second indication indicating whether the second sensor detected at least one label in the fluid chamber;
cleaving a second linker; and
after cleaving the second linker, repeating (d) through (i).

81. The method recited in claim 70, wherein the sequencing procedure comprises:
(a) introducing a labeled nucleotide into the fluid chamber;
(b) rinsing away unbound molecules;
(c) obtaining a first characteristic from a first sensor;
(d) obtaining a second characteristic from a second sensor;
(e) determining, based on the first characteristic, whether the first sensor detected at least one label in the fluid chamber;
(f) determining, based on the second characteristic, whether the second sensor detected at least one label in the fluid chamber;
(g) recording a first indication in a first record of the S records, the first indication indicating whether the first sensor detected at least one label in the fluid chamber;
(h) recording a second indication in a second record of the S records, the second indication indicating whether the second sensor detected at least one label in the fluid chamber;
(i) cleaving and rinsing away labels; and
after cleaving and rinsing away labels, repeating (a) through (i) for at least one other labeled nucleotide.

82. The method recited in claim 70, wherein a number of records in the at least a subset of the S records is odd.

83. (canceled)

84. The method recited in claim 70, wherein applying the error correction procedure comprises:
identifying, in at least one record of the at least a subset of the S records, a run of second binary values, and
deleting the run of the second binary values from the at least one record.

85. (canceled)

86. The method recited in claim 70, wherein the sequencing procedure comprises (a) a first inquiry step, (b) a label-removal step to remove the labels present in the fluid chamber after the first inquiry step, (c) a sensing step to detect residual labels present in the fluid chamber after the label-removal step, and (d) a second inquiry step after the sensing step, and wherein performing the error correction procedure comprises:
in response to determining, via the sensing step, that a particular sensor of the S sensors detects a residual label in the fluid chamber, recording the second binary value in a particular position of a particular record of the S records, the particular record capturing the detection results of the particular sensor, wherein the particular position captures a result of the second inquiry step.

87. The method recited in claim 70, wherein applying the error correction procedure comprises:
identifying, in at least one record of the at least a subset of the S records, a set of consecutive indications that no label was detected, and
deleting the set of consecutive indications that no label was detected from the at least one record.

88. The method recited in claim 70, wherein applying the error correction procedure comprises modifying one or more of the at least a subset of the S records.

89. The method recited in claim 70, wherein the at least a subset of the S records comprises an odd number of at least three records representing sequencing results of instances of a first nucleic acid strand.

90. The method recited in claim 89, wherein applying the error correction procedure comprises:
identifying, in each of the at least a subset of the S records, a majority detection result for a particular inquiry step; and
calling or not calling a base of the first nucleic acid strand based at least in part on the majority detection result.

91. The method recited in claim 89, wherein the at least a subset of the S records consists of first, second, and third records, and wherein applying the error correction procedure comprises, for a selected detection result of the M detection results:
in response to the selected detection result in at least two of the first, second, and third records being identical, recording a base of the first nucleic acid strand based at least in part on the identical selected detection result.

92. The method recited in claim 70, wherein applying the error correction procedure comprises, for a selected detection result of the M detection results:
in response to the selected detection result in more than half of the at least a subset of the S records being identical, calling or not calling a base of the at least one of the S nucleic acid strands based at least in part on the identical selected detection result.

93. The method recited in claim 70, wherein applying the error correction procedure comprises, for a selected detection result of the M detection results:
in response to the selected detection result in more than half of the at least a subset of the S records indicating detection of the at least one label in the fluid chamber, calling a base of the at least one of the S nucleic acid strands.

94-95. (canceled)

96. A method of mitigating errors in sequencing data generated as a result of a nucleic acid sequencing procedure using a single-molecule sensor array, the single-molecule sensor array having a plurality of sensors, each of the plurality of sensors associated with a respective binding site of a plurality of binding sites, each of the plurality of binding sites configured to bind no more than one strand of nucleic acid to be sequenced, the method comprising:
identifying, in the sequencing data, a plurality of records, each of the plurality of records capturing a respective sequencing result for a respective instance of a first strand of nucleic acid, each of the plurality of records having a plurality of entries, each of the plurality of entries indicating, for a respective one of a plurality of inquiry steps of the nucleic acid sequencing procedure, that either (a) a label was detected by a respective sensor associated with the respective instance of the first strand of nucleic acid, or (b) no label was detected by the respective sensor associated with the respective instance of the first strand of nucleic acid;
based on the plurality of records, determining a plurality of candidate sequences for the first strand of nucleic acid, each of the plurality of candidate sequences estimating at least a portion of a nucleic acid sequence of the first strand of nucleic acid; and
identifying, as the at least a portion the nucleic acid sequence of the first strand of nucleic acid, a particular candidate sequence of the plurality of candidate sequences that is, from among the plurality of candidate sequences, most likely to be correct.

97. The method recited in claim 96, wherein identifying the plurality of records comprises at least one of:
(a) searching the sequencing data for a barcode associated with the first strand of nucleic acid, or
(b) identifying a common sequence of entries in each of the plurality of records.

98. (canceled)

99. The method recited in claim 96, wherein the at least a portion of the nucleic acid sequence of the first strand of nucleic acid is a single base.

100. The method recited in claim 96, wherein determining the plurality of candidate sequences for the first strand of nucleic acid comprises:
identifying within the plurality of records a particular inquiry step at which a first sensor detected a respective label and a second sensor did not detect any label;
establishing a first candidate sequence that assumes the first sensor correctly detected the respective label; and
establishing a second candidate sequence that assumes the first sensor incorrectly detected the respective label.

101. The method recited in claim 96, wherein determining the plurality of candidate sequences for the first strand of nucleic acid comprises:
identifying within the plurality of records a particular inquiry step at which a first sensor detected a respective label and a second sensor did not detect any label;
establishing a first candidate sequence that assumes the second sensor incorrectly failed to detect any label; and
establishing a second candidate sequence that assumes the second sensor correctly failed to detect any label.

102. The method recited in claim 96, wherein each of the plurality of entries is a first binary value or a second binary value, wherein the first binary value indicates that the label was detected by the respective sensor, and the second binary value indicates that no label was detected by the respective sensor, and wherein determining the plurality of candidate sequences for the first strand of nucleic acid comprises:
identifying, in at least one of the plurality of records, a run of second binary values, and deleting the run of the second binary values from the at least one of the plurality of records.

103. (canceled)

104. The method recited in claim 96, wherein determining the plurality of candidate sequences for the first strand of nucleic acid comprises:
identifying, in at least one of the plurality of records, a set of consecutive entries indicating that no label was detected, and
deleting the set of consecutive entries indicating that no label was detected from the at least one of the plurality of records.

105. The method recited in claim 96, wherein identifying the particular candidate sequence of the plurality of candidate sequences that is most likely to be correct comprises determining or estimating which of the plurality of candidate sequences has a highest probability of being correct.

106. The method recited in claim 96, wherein the at least a portion of the nucleic acid sequence of the first strand of nucleic acid is a single base, and wherein identifying the particular candidate sequence of the plurality of candidate sequences that is most likely to be correct comprises identifying a majority result for a particular inquiry step represented by the plurality of records.

107. The method recited in claim 96, wherein identifying the particular candidate sequence of the plurality of candidate sequences that is most likely to be correct comprises:
determining, for each of the plurality of candidate sequences, a respective likelihood of occurrence; and
choosing the particular candidate sequence based on its respective likelihood of occurrence meeting a constraint.

108. The method recited in claim 107, wherein the constraint is a minimum probability.

109. The method recited in claim 107, wherein the constraint is that the respective likelihood of occurrence of the particular candidate sequence is higher than the respective likelihoods of occurrence of all other candidate sequences of the plurality of candidate sequences.

110. The method recited in claim 96, wherein identifying the particular candidate sequence of the plurality of candidate sequences that is most likely to be correct comprises eliminating at least one of the plurality of candidate sequences based on a known constraint on a nucleic acid sequence of the first strand of nucleic acid.

111. The method recited in claim 110, wherein the known constraint is an impossibility of a particular sequence of bases.

112. The method recited in claim 110, further comprising determining the known constraint based at least in part on a source of the first strand of nucleic acid.


SEQUENCE LISTING (16 sequences)

SEQ ID NO: 1 - DNA, 27 bp, Artificial Sequence
  Description of Artificial Sequence Synthetic oligonucleotide
tagcaaggtccgctactggcagactgg

SEQ ID NO: 2 - DNA, 20 bp, Artificial Sequence
  Description of Artificial Sequence Synthetic oligonucleotide
tagcaaggtccgctactggc

SEQ ID NO: 3 - DNA, 21 bp, Artificial Sequence
  Description of Artificial Sequence Synthetic oligonucleotide
tagcaagtgtccgctactggc

SEQ ID NO: 4 - DNA, 20 bp, Artificial Sequence
  Description of Artificial Sequence Synthetic oligonucleotide
tagcaagtgtccctactggc

SEQ ID NO: 5 - DNA, 21 bp, Artificial Sequence
  Description of Artificial Sequence Synthetic oligonucleotide
tatgcaagggtccgtacttcg

SEQ ID NO: 6 - DNA, 27 bp, Artificial Sequence
  Description of Artificial Sequence Synthetic oligonucleotide
tagcaaggtcggctactggcagactgg

SEQ ID NO: 7 - DNA, 16 bp, Artificial Sequence
  Description of Artificial Sequence Synthetic oligonucleotide
tagcaaggtcctactg

SEQ ID NO: 8 - DNA, 20 bp, Artificial Sequence
  Description of Artificial Sequence Synthetic oligonucleotide
tagcaaggtccgctactggc

SEQ ID NO: 9 - DNA, 18 bp, Artificial Sequence
  Description of Artificial Sequence Synthetic oligonucleotide
tagcaaggtccgctactg

SEQ ID NO: 10 - DNA, 18 bp, Artificial Sequence
  Description of Artificial Sequence Synthetic oligonucleotide
tagcaaggcccccgggac

SEQ ID NO: 11 - DNA, 22 bp, Artificial Sequence
  Description of Artificial Sequence Synthetic oligonucleotide
tagcaaggtccgctactggcag

SEQ ID NO: 12 - DNA, 17 bp, Artificial Sequence
  Description of Artificial Sequence Synthetic oligonucleotide
tagcaagtgtcctactg

SEQ ID NO: 13 - DNA, 20 bp, Artificial Sequence
  Description of Artificial Sequence Synthetic oligonucleotide
tagcaagtgtcctcctggcg

SEQ ID NO: 14 - DNA, 21 bp, Artificial Sequence
  Description of Artificial Sequence Synthetic oligonucleotide
tagcaagtgtccgctactggc

SEQ ID NO: 15 - DNA, 17 bp, Artificial Sequence
  Description of Artificial Sequence Synthetic oligonucleotide
tagcaagtgttcacttg

SEQ ID NO: 16 - DNA, 20 bp, Artificial Sequence
  Description of Artificial Sequence Synthetic oligonucleotide
tagcaagtgtccctactggc